(19) United States
(12) Patent Application Publication
(10) Pub. No.: US 2010/0255002 A1
(43) Pub. Date: Oct. 7, 2010

(54) Immunogenic compositions for chlamydia trachomatis

(75) Inventors: Guido Grandi, Emeryville, CA (US); Oretta Finco, Emmeryville, CA (US); Giulio Ratti, Emmeryville, CA (US); Alessandro Bonci, Emmeryville, CA (US)

Correspondence Address: NOVARTIS VACCINES AND DIAGNOSTICS INC., INTELLECTUAL PROPERTY- X100B, P.O. BOX 8097, Emeryville, CA, 94662-8097, US

(73) Assignee: CHIRON CORPORATION, EMMERYVILLE CA (US)

(21) Appl. No.: 10/561,236

(22) PCT Filed: Jun. 25, 2004
(86) PCT No.: PCT/US04/20491

§ 371 Date: Feb. 18, 2008

Related U.S. Application Data
(60) Provisional application No. 60/497,649, filed on Aug. 25, 2003.
(60) Provisional application No. 60/576,375, filed on Jun. 1, 2004.

(30) Foreign Application Priority Data
Jun. 26, 2003 (GB) 0315020.8
Feb. 2, 2004 (GB) 0402236.4

(51) Int. Cl. A61K 39/40; A61K 39/118; A61K 39/39; A61P 31/04

(52) U.S. Cl. 424/164.1; 424/263.1

(57) ABSTRACT

The invention relates to compositions comprising combinations of Chlamydia trachomatis antigens and their use in vaccines. Specific combinations may be selected from a first antigen group of PepA, LcrE, ArtJ, DnaK, and CT398, and a second antigen group of PepA, LcrE, ArtJ, DnaK, CT398, OmpH-like, L7/L12, OmcA, AtoS, CT547, Eno, HtrA and MurG. The invention further relates to the use of combinations of adjuvants for use with antigens associated with a sexually transmissible disease, such as Chlamydia trachomatis antigens. Preferred adjuvant combinations include mineral salts, such as aluminium salts and oligonucleotides comprising a CpG motif.

### CROSS REFERENCE TO RELATED APPLICATIONS, FROM WHICH PRIORITY IS CLAIMED

This application incorporates by reference in its entirety United Kingdom patent application No. 0315020.8, filed on Jun. 26, 2003; U.S. Provisional patent application Ser. No. 60/497,649, filed on Aug. 25, 2003; United Kingdom patent application No. 0402236.4, filed on Feb. 2, 2004, and U.S. Provisional patent application Ser. No. 60/576,375, filed on Jun. 1, 2004.

### FIELD OF THE INVENTION

This invention is in the fields of immunology and vaccinology. In particular, it relates to antigens derived from Chlamydia trachomatis and their use in immunisation.

### BACKGROUND OF THE INVENTION

The Chlamydiae are obligate intracellular parasites of eukaryotic cells which are responsible for endemic sexually transmitted infections and various other disease syndromes. They occupy an exclusive eubacterial phylogenic branch, having no close relationship to any other known organisms.

Historically, the Chlamydiae have been classified in their own order (Chlamydiales) made up of a single family (Chlamydiaceae) which in turn contains a single genus (Chlamydia, also referred to as Chlamydophila). More recently, this order has been divided into at least four families including Chlamydiaceae, Parachlamydiaceae, Waddiaceae and Simkaniaceae. In this more recent classification, the Chlamydiaceae family includes genuses of Chlamydophila and Chlamydia, Chlamydia trachomatis being a species within the Chlamydia genus. See, Bush et al., (2001) Int. J. Syst. Evol. Microbial. 51:203-220.

A particular characteristic of the Chlamydiae is their unique life cycle, in which the bacterium alternates between two morphologically distinct forms: an extracellular infective form (elementary bodies, EB) and an intracellular non-infective form (reticulate bodies, RB). The life cycle is completed with the re-organization of RB into EB, which leave the disrupted host cell ready to infect further cells.

The genome sequences of at least five chlamydia or chlamydophila species are currently known—C. trachomatis, C. pneumoniae, C. muridarum, C. pecorum and C. psittaci (See Kalman et al., (1999) Nature Genetics 21:385-389; Read et al. (2000) Nucleic Acids Res. 28:1397-1406; Shirai et al. (2000) Nucleic Acids Res 28:2311-2314; Stephens et al. (1998) Science 282:754-759; and International patent publications WO99/27105, WO00/27994 and WO99/28475).

The human serovariants (“serovars”) of C. trachomatis are divided into two biovariants (“biovars”). Serovars A-K elicit epithelial infections primarily in the ocular tissue (A-C) or urogenital tract (D-K). Serovars L1, L2 and L3 are the agents of invasive lymphogranuloma venereum (LGV).

Although chlamydial infection itself causes disease, it is thought that the severity of symptoms in some patients is actually due to an aberrant host immune response. Failure to clear the infection results in persistent immune stimulation and, rather than helping the host, this results in chronic infection with severe consequences, including sterility and blindness. See, e.g., Ward, (1995) Apmis. 103:769-96. In addition, the protection conferred by natural chlamydial infection is usually incomplete, transient, and strain-specific.

More than 4 million new cases of chlamydial sexually transmitted infections are diagnosed each year in the United States alone and the cost of their treatment has been estimated in 4 billion dollars annually, with 80% attributed to infection and disease of women. Although chlamydial infections can be treated with several antibiotics, a majority of the female infections are asymptomatic, and antimicrobial therapy may be delayed or inadequate to prevent long term sequelae, especially in countries with poor hygienic conditions. Multiple-antibiotic-resistant strains of Chlamydia have also been reported (Somani, et al., 2000). Furthermore it has been suggested that antibiotic treatment could lead to the formation of aberrant forms of C. trachomatis that maybe reactivated later on (See, Hammerschlag M. R., (2002) Semin. Pediatr. Infect. Dis. 13:239-248).

Unfortunately the major determinants of chlamydial pathogenesis are complicated and at present still unclear, mostly due to the intrinsic difficulty in working with this pathogen and the lack of adequate methods for its genetic manipulation. In particular very little is known about the antigenic composition of elementary body surface, that is an essential compartment in pathogen-host interactions, and likely to carry antigens able to elicit a protective immune response.

Due to the serious nature of the disease, there is a desire to provide suitable vaccines. These may be useful (a) for immunisation against chlamydial infection or against chlamydia-induced disease (prophylactic vaccination) or (b) for the eradication of an established chronic chlamydial infection (therapeutic vaccination). Being an intracellular parasite, however, the bacterium can generally evade antibody-mediated immune responses.

Various antigenic proteins have been described for C. trachomatis, and the cell surface in particular has been the target of detailed research. See, e.g., Moulder (1991) Microbiol Rev 55(1):143-190. These include, for instance, Pgp3, MOMP, Hsp60 (GroEL) and Hsp70 (Dna-K like). References describing Pgp3 include Comanducci et al. (1994) Infect Immun 62(12):5491-5497 and patent publications EP 0499681 and WO95/28487). References describing MOMP include Murdin et al. (1993) Infect Immun 61:4406-4414. References describing Hsp60 (GroEL) include Cerrone et al. (1991) Infect Immun 59(1):79-90). References describing Hsp70 (DnaK-like) include Raulston et al. (1993) J. Biol. Chem. 268:23139-23147). Not all of these have proved to be effective vaccines, however, and further candidates have been identified. See WO03/049762.

Vaccines against pathogens such as hepatitis B virus, diphtheria and tetanus typically contain a single protein antigen (e.g. the HBV surface antigen, or a tetanus toxoid). In contrast, acellular whooping cough vaccines typically have at least three B. pertussis proteins, and the Prevnar™ pneumococcal vaccine contains seven separate conjugated saccharide antigens. Other vaccines such as cellular pertussis vaccines, the measles vaccine, the inactivated polio vaccine (IPV) and meningococcal OMV vaccines are by their very nature complex mixtures of a large number of antigens. Whether protection can be elicited by a single antigen, a small number of defined antigens, or a complex mixture of undefined antigens, therefore depends on the pathogen in question.

It is an object of the invention to provide further and improved compositions for providing immunity against chlamydial disease and/or infection. The compositions are based on a combination of two or more (e.g. three or more) C. trachomatis antigens. In addition, the compositions may also be based on the use of C. trachomatis antigens with a combination of adjuvants designed to elicit an enhanced immune response. Preferably, the combination of adjuvants comprises an aluminium salt and an oligonucleotide comprising a CpG motif.

### SUMMARY OF THE INVENTION

Within the ˜900 proteins previously described for the C. trachomatis genome (See e.g., Stephens et al. (1998) Science 282:754-759), Applicants have discovered a group of five Chlamydia trachomatis antigens that are particularly suitable for immunisation purposes, particularly when used in combinations. The invention therefore provides a composition comprising a combination of Chlamydia trachomatis antigens, said combination consisting of two, three, four or all five Chlamydia trachomatis antigens of a first antigen group, said first antigen group consisting of: (1) PepA (CT045); (2) LcrE (CT089); (3) ArtJ (CT381); (4) DnaK (CT396); and (5) CT398. These antigens are referred to herein as the ‘first antigen group’. Preferably the combination includes LcrE (CT089).

The invention also provides for a slightly larger group of 13 Chlamydia trachomatis antigens that are particularly suitable for immunisation purposes, particularly when used in combinations. (This second antigen group includes the five Chlamydia trachomatis antigens of the first antigen group.) These 13 Chlamydia trachomatis antigens form a second antigen group of (1) PepA (CT045); (2) LcrE (CT089); (3) ArtJ (CT381); (4) DnaK (CT396); (5) CT398; (6) OmpH-like (CT242); (7) L7/L12 (CT316); (8) OmcA (CT444); (9) AtoS (CT467); (10) CT547; (11) Eno (CT587); (12) HtrA (CT823) and (13) MurG (CT761). These antigens are referred to herein as the ‘second antigen group’. Preferably, the combination includes one or more of LcrE (CT089) and OmpH-like protein (CT242).

The invention therefore provides a composition comprising a combination of Chlamydia trachomatis antigens, said combination selected from the group consisting of two, three, four, five, six, seven, eight, nine, ten, eleven, twelve, or thirteen Chlamydia trachomatis antigens of the second antigen group. Preferably, the combination is selected from the group consisting of two, three, four or five Chlamydia trachomatis antigens of the second antigen group. Still more preferably, the combination consists of five Chlamydia trachomatis antigens of the second antigen group.

The compositions of the invention may comprise one or more immunoregulatory agents. Such immunoregulatory agents include adjuvants. Preferably, the adjuvants are selected from the group consisting of a TH1 adjuvant and a TH2 adjuvant. Still more preferably, the adjuvants are selected from the group consisting of aluminium salts and oligonucleotides comprising a CpG motif. The invention therefore provides a composition comprising a Chlamydia trachomatis antigen, or an antigen associated with a sexually transmissible disease, an oligonucleotide containing a CpG motif and a mineral salt, such as an aluminium salt.

### BRIEF DESCRIPTION OF THE DRAWINGS

FIG. 1 depicts a western blot analysis of total protein extracts from C. trachomatis EBs, performed using mouse immune sera against recombinant antigens. Only FACS positive non neutralizing sera are shown. For antigen identification, please see Table 1(a). The panel identification numbers correspond to the numbers reported in the WB analysis column of Table 1(a). In each panel, the strip on the right shows the results obtained with the antigen-specific immune serum (I), and the strip on the left shows the results obtained with the corresponding preimmune serum (P).

FIG. 2 illustrates serum titres giving 50% neutralization of infectivity for the 9 C. trachomatis recombinant antigens described in the text (PepA, ArtJ, DnaK, CT398, CT547, Enolase, MOMP, OmpH-like and AtoS. Each titre was assessed in 3 separate experiments (SEM values shown).

FIG. 3 includes FACS analysis of antibody binding to whole C. trachomatis EBs. Gray histograms (event counts versus fluorescence channels) are the FACS output for EBs stained with background control antibodies. White histograms are the FACS output of EBs stained with antigen-specific antibodies. Positive control was represented by an anti-C. trachomatis mouse hyperimmune serum against whole EBs, with the corresponding preimmune mouse serum as background control; Negative controls were obtained by staining EBs with either mouse anti-GST or mouse anti-HIS hyperimmune serum, with the corresponding preimmune serum as background control. For each serum the background control was represented by mouse anti-GST or mouse anti-HIS hyperimmune serum, depending on the fusion protein used for immunization. Western blotting data obtained from total EB proteins stained with the same antiserum used for the FACS assays are also shown within each panel.

FIG. 4 shows a Faster Clearance of Chlamydia trachomatis (CT) at 21 days post-challenge in mice vaccinated with a mixture of CT242 (OmpH-like) and CT316 (L7/L12) in combination with CFA when compared with the mice vaccinated with CFA alone.

FIG. 5 shows a Faster Clearance of Chlamydia trachomatis (CT) at 21 days post-challenge in mice vaccinated with a mixture of CT467 (AtoS) and CT444 (OmcA) in combination with CFA when compared with CT clearance in mice vaccinated with CFA alone.

FIG. 6 shows a Faster Clearance of Chlamydia trachomatis (CT) at 21 days post-challenge in mice vaccinated with a mixture of CT812 (PmpD) and CT082 (Hypothetical) in combination with CFA when compared with CT clearance in mice vaccinated with CFA alone.

FIGS. 7(a) and 7(b) show a statistically significant clearance of Chlamydia trachomatis at 14 days post-challenge in mice vaccinated with a mixture of CT242 and CT316 in combination with CFA when compared with CT clearance in mice vaccinated with CFA alone.

FIG. 7(c) shows the neutralization titre for mice vaccinated with a mixture of CT242 and CT316 in combination with CFA.

FIGS. 8(a) and 8(b) show a clearance of Chlamydia trachomatis at 14 days post-challenge in mice vaccinated with a mixture of five CT antigens, these being CT 045, CT089, CT396, CT398 and CT381 in combination with AlOH and CpG when compared with CT clearance in mice vaccinated with AlOH and CpG alone.

FIG. 8(c) shows the Chlamydia specific IgG antibody isotypes (IgG1 and IgG2a) for pre-challenge sera from (i) mice vaccinated with a mixture of five CT antigens, these being CT045, CT089, CT396, CT398 and CT381 in combination with AlOH and CpG and (ii) mice vaccinated with a mixture of five CT antigens, these being CT045, CT089, CT396, CT398 and CT381 in combination with CFA.

FIGS. 9(a) and 9(b) show the clearance of Chlamydia trachomatis (CT) at 7, 14 and 21 days post-challenge in mice vaccinated with a mixture of five CT antigens, these being CT 045, CT089, CT396, CT398 and CT381 in combination with AlOH and CpG when compared with CT clearance in mice vaccinated with AlOH and CpG alone.

FIG. 9(c) shows the neutralization titre and Chlamydia specific IgG antibody isotypes (IgG1 and IgG2) for pre-challenge sera from mice vaccinated with a mixture of five CT antigens, these being CT 045, CT089, CT396, CT398 and CT381 in combination with AlOH and CpG.

FIGS. 10(a) and (b) show the neutralization titre for mice vaccinated with a mixture of five CT antigens, these being CT 045, CT089, CT396, CT398 and CT381 in combination with AlOH and CpG compared with the serum neutralization titre obtained for mice vaccinated with AlOH and CpG alone.

### DETAILED DESCRIPTION OF THE INVENTION

As discussed above, the invention provides compositions comprising a combination of Chlamydia trachomatis antigens, wherein the combinations can be selected from groups of antigens which Applicants have identified as being particularly suitable for immunization purposes, particularly when used in combination. In one embodiment, the invention provides a composition comprising a combination of Chlamydia trachomatis antigens, said combination consisting of two, three, four or all five Chlamydia trachomatis antigens of a first antigen group, said first antigen group consisting of: (1) PepA (CT045); (2) LcrE (CT089); (3) ArtJ (CT381); (4) DnaK (CT396); and (5) CT398. These antigens are referred to herein as the ‘first antigen group’.

Preferably, the composition of the invention comprises a combination of Chlamydia trachomatis antigens, said combination selected from the group consisting of: (1) PepA & LcrE; (2) PepA & ArtJ; (3) PepA & DnaK; (4) PepA & CT398; (5) LcrE & ArtJ; (6) LcrE & DnaK; (7) LcrE & CT398; (8) ArtJ & DnaK; (9) ArtJ & CT398; (10) DnaK & CT398; (11) PepA, LcrE & ArtJ; (12) PepA, LcrE & DnaK; (13) PepA, LcrE & CT398; (14) PepA, ArtJ & DnaK; (15) PepA, ArtJ and CT398; (16) PepA, DnaK & CT398; (17) LcrE, ArtJ & DnaK; (18) LcrE, ArtJ & CT398; (19) LcrE, DnaK & CT398; (20) ArtJ, DnaK & CT398; (21) PepA, LcrE, ArtJ & DnaK; (22) PepA, LcrE, DnaK & CT398; (23) PepA, ArtJ, DnaK & CT398; (24) PepA, LcrE, ArtJ & CT398; (25) LcrE, ArtJ, DnaK & CT398; and (26) PepA, LcrE, ArtJ, DnaK & CT398. Preferably, the composition of Chlamydia trachomatis antigens consists of PepA, LcrE, ArtJ, DnaK & CT398. Preferably, the combination includes LcrE (CT089).

The invention also provides for a slightly larger group of 13 Chlamydia trachomatis antigens that are particularly suitable for immunisation purposes, particularly when used in combinations. (This second antigen group includes the five Chlamydia trachomatis antigens of the first antigen group.) These 13 Chlamydia trachomatis antigens form a second antigen group of (1) PepA (CT045); (2) LcrE (CT089); (3) ArtJ (CT381); (4) DnaK (CT396); (5) CT398; (6) OmpH-like (CT242); (7) L7/L12 (CT316); (8) OmcA (CT444); (9) AtoS (CT467); (10) CT547; (11) Eno (CT587); (12) HtrA (CT823) and (13) MurG (CT761). These antigens are referred to herein as the ‘second antigen group’.

The invention therefore provides a composition comprising a combination of Chlamydia trachomatis antigens, said combination selected from the group consisting of two, three, four, five, six, seven, eight, nine, ten, eleven, twelve, or thirteen Chlamydia trachomatis antigens of the second antigen group. Preferably, the combination is selected from the group consisting of two, three, four or five Chlamydia trachomatis antigens of the second antigen group. Still more preferably, the combination consists of five Chlamydia trachomatis antigens of the second antigen group. Preferably, the combination includes one or both of LcrE (CT089) and OmpH-like protein (CT242).

Each of the Chlamydia trachomatis antigens of the first and second antigen group are described in more detail below.

(1) PepA leucyl aminopeptidase A protein (CT045) One example of a ‘PepA’ protein is disclosed as SEQ ID NO^{s}: 71 & 72 in WO 03/049762 (GenBank accession number: AAC67636, GI:3328437; ‘CT045’; SEQ ID NO: 1 in attached sequence listing). It is believed to catalyse the removal of unsubstituted N-terminal amino acids from various polypeptides. Preferred PepA proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 1; and/or (b) which is a fragment of at least n consecutive amino acids of SEQ ID NO: 1, wherein n is 7 or more (e.g. 8, 10, 12, 14, 16, 18, 20, 25, 30, 35, 40, 50, 60, 70, 80, 90, 100, 150, 200, 250 or more). These PepA proteins include variants (e.g. allelic variants, homologs, orthologs, paralogs, mutants, etc.) of SEQ ID NO: 1. Preferred fragments of (b) comprise an epitope from SEQ ID NO: 1. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 1. Other fragments omit one or more domains of the protein (e.g. omission of a signal peptide, of a cytoplasmic domain, of a transmembrane domain, or of an extracellular domain). The PepA protein may contain manganese ions.

(2) LcrE low calcium response E protein (CT089) One example of a ‘LcrE’ protein is disclosed as SEQ ID NO^{s}: 61 & 62 in WO 03/049762 (GenBank accession number: AAC67680, GI:3328485; ‘CT089’; SEQ ID NO: 2 in attached sequence listing). Preferred LcrE proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 2; and/or (b) which is a fragment of at least n consecutive amino acids of SEQ ID NO: 2, wherein n is 7 or more (e.g. 8, 10, 12, 14, 16, 18, 20, 25, 30, 35, 40, 50, 60, 70, 80, 90, 100, 150, 200, 250 or more). These LcrE proteins include variants (e.g. allelic variants, homologs, orthologs, paralogs, mutants, etc.) of SEQ ID NO: 2. Preferred fragments of (b) comprise an epitope from SEQ ID NO: 2. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 2. Other fragments omit one or more domains of the protein (e.g. omission of a signal peptide, of a cytoplasmic domain, of a transmembrane domain, or of an extracellular domain).

(3) ArtJ arginine-binding protein (CT381) One example of ‘ArtJ’ protein is disclosed as SEQ ID NO^{s}: 105 & 106 in WO 03/049762 (GenBank accession number: AAC67977, GI:3328806; ‘CT381’; SEQ ID NO: 3 in attached sequence listing). Preferred ArtJ proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 3; and/or (b) which is a fragment of at least n consecutive amino acids of SEQ ID NO: 3, wherein n is 7 or more (e.g. 8, 10, 12, 14, 16, 18, 20, 25, 30, 35, 40, 50, 60, 70, 80, 90, 100, 150, 200, 250 or more). These ArtJ proteins include variants (e.g. allelic variants, homologs, orthologs, paralogs, mutants, etc.) of SEQ ID NO: 3. Preferred fragments of (b) comprise an epitope from SEQ ID NO: 3. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 3. Other fragments omit one or more domains of the protein (e.g. omission of a signal peptide, of a cytoplasmic domain, of a transmembrane domain, or of an extracellular domain). The ArtJ protein may be bound to a small molecule like arginine or another amino acid.

(4) DnaK heat-shock protein 70 (chaperone) (CT396) One example of ‘DnaK’ protein is disclosed as SEQ ID NO^{s}: 107 & 108 in WO 03/049762 (GenBank accession number: AAC67993, GI:3328822; ‘CT396’; SEQ ID NO: 4 in attached sequence listing). Other sequences are disclosed in Birkelund et al. (1990) Infect Immun 58:2098-2104; Danilition et al. (1990) Infect Immun 58:189-196; and Raulston et al. (1993) J Biol Chem 268:23139-23147. Preferred DnaK proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 4; and/or (b) which is a fragment of at least n consecutive amino acids of SEQ ID NO: 4, wherein n is 7 or more (e.g. 8, 10, 12, 14, 16, 18, 20, 25, 30, 35, 40, 50, 60, 70, 80, 90, 100, 150, 200, 250 or more). These DnaK proteins include variants (e.g. allelic variants, homologs, orthologs, paralogs, mutants, etc.) of SEQ ID NO: 4. Preferred fragments of (b) comprise an epitope from SEQ ID NO: 4. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 4. Other fragments omit one or more domains of the protein (e.g. omission of a signal peptide, of a cytoplasmic domain, of a transmembrane domain, or of an extracellular domain). The DnaK may be phosphorylated e.g. at a threonine or a tyrosine.

(5) CT398 protein (Hypothetical Protein) One example of ‘CT398’ protein is disclosed as SEQ ID NO^{s}: 111 & 112 in WO 03/049762 (GenBank accession number: AAC67995, GI:3328825; SEQ ID NO: 5 in attached sequence listing). Preferred CT398 proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 5; and/or (b) which is a fragment of at least n consecutive amino acids of SEQ ID NO: 5, wherein n is 7 or more (e.g. 8, 10, 12, 14, 16, 18, 20, 25, 30, 35, 40, 50, 60, 70, 80, 90, 100, 150, 200, 250 or more). These CT398 proteins include variants (e.g. allelic variants, homologs, orthologs, paralogs, mutants, etc.) of SEQ ID NO: 5. Preferred fragments of (b) comprise an epitope from SEQ ID NO: 5. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 5. Other fragments omit one or more domains of the protein (e.g. omission of a signal peptide, of a cytoplasmic domain, of a transmembrane domain, or of an extracellular domain).

(6) OmpH-like outer membrane protein (CT242) One example of ‘OmpH-like’ protein is disclosed as SEQ ID NO^{s}: 57 & 58 in WO 03/049762 (GenBank accession number: AAC67835, GI:3328652; ‘CT242’; SEQ ID NO: 6 in attached sequence listing). A variant sequence is disclosed in Bannantine & Rockey (1999) Microbiology 145:2077-2085. Preferred OmpH-like proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 6; and/or (b) which is a fragment of at least n consecutive amino acids of SEQ ID NO: 6, wherein n is 7 or more (e.g. 8, 10, 12, 14, 16, 18, 20, 25, 30, 35, 40, 50, 60, 70, 80, 90, 100, 150, 200, 250 or more). These OmpH-like proteins include variants (e.g. allelic variants, homologs, orthologs, paralogs, mutants, etc.) of SEQ ID NO: 6. Preferred fragments of (b) comprise an epitope from SEQ ID NO: 6. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more; preferably 19 or more, to remove the signal peptide) from the N-terminus of SEQ ID NO: 6. Other fragments omit one or more domains of the protein (e.g. omission of a signal peptide as described above, of a cytoplasmic domain, of a transmembrane domain, or of an extracellular domain).

(7) L7/L12 ribosomal protein (CT316) One example of ‘L7/L12’ protein is deposited in GenBank under accession number AAC67909 (GI:3328733; ‘CT316’; SEQ ID NO: 7 in attached sequence listing). Preferred L7/L12 proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 7; and/or (b) which is a fragment of at least n consecutive amino acids of SEQ ID NO: 7, wherein n is 7 or more (e.g. 8, 10, 12, 14, 16, 18, 20, 25, 30, 35, 40, 50, 60, 70, 80, 90, 100, 150, 200, 250 or more). These L7/L12 proteins include variants (e.g. allelic variants, homologs, orthologs, paralogs, mutants, etc.) of SEQ ID NO: 7. Preferred fragments of (b) comprise an epitope from SEQ ID NO: 7. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 7. Other fragments omit one or more domains of the protein (e.g. omission of a signal peptide, of a cytoplasmic domain, of a transmembrane domain, or of an extracellular domain). The L7/L12 protein may be N-terminally modified.

(8) OmcA cysteine-rich lipoprotein (CT444) One example of ‘OmcA’ protein is disclosed as SEQ ID NO^{s}: 127 & 128 in WO 03/049762 (GenBank accession number: AAC68043, GI:3328876; ‘CT444’, ‘Omp2A’, ‘Omp3’; SEQ ID NO: 8 in attached sequence listing). A variant sequence is disclosed in Allen et al. (1990) Mol. Microbiol. 4:1543-1550. Preferred OmcA proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 8; and/or (b) which is a fragment of at least n consecutive amino acids of SEQ ID NO: 8, wherein n is 7 or more (e.g. 8, 10, 12, 14, 16, 18, 20, 25, 30, 35, 40, 50, 60, 70, 80, 90, 100, 150, 200, 250 or more). These OmcA proteins include variants (e.g. allelic variants, homologs, orthologs, paralogs, mutants, etc.) of SEQ ID NO: 8. Preferred fragments of (b) comprise an epitope from SEQ ID NO: 8. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more; preferably 18 or more to remove the signal peptide) from the N-terminus of SEQ ID NO: 8. Other fragments omit one or more domains of the protein (e.g. omission of a signal peptide as described above, of a cytoplasmic domain, of a transmembrane domain, or of an extracellular domain). The protein may be lipidated (e.g. by a N-acyl diglyceride), and may thus have a N-terminal cysteine.

(9) AtoS two-component regulatory system sensor histidine kinase protein (CT467) One example of ‘AtoS’ protein is disclosed as SEQ ID NO^{s}: 129 & 130 in WO 03/049762 (GenBank accession number: AAC68067, GI:3328901; ‘CT467’; SEQ ID NO: 9 in attached sequence listing). Preferred AtoS proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 9; and/or (b) which is a fragment of at least n consecutive amino acids of SEQ ID NO: 9, wherein n is 7 or more (e.g. 8, 10, 12, 14, 16, 18, 20, 25, 30, 35, 40, 50, 60, 70, 80, 90, 100, 150, 200, 250 or more). These AtoS proteins include variants (e.g. allelic variants, homologs, orthologs, paralogs, mutants, etc.) of SEQ ID NO: 9. Preferred fragments of (b) comprise an epitope from SEQ ID NO: 9. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 9. Other fragments omit one or more domains of the protein (e.g. omission of a signal peptide, of a cytoplasmic domain, of a transmembrane domain, or of an extracellular domain).

(10) CT547 protein (Hypothetical Protein) One example of ‘CT547’ protein is disclosed as SEQ ID NO^{s}: 151 & 152 in WO 03/049762 (GenBank accession number: AAC67995, GI:3328825; SEQ ID NO: 10 in attached sequence listing). Preferred CT547 proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 10; and/or (b) which is a fragment of at least n consecutive amino acids of SEQ ID NO: 10, wherein n is 7 or more (e.g. 8, 10, 12, 14, 16, 18, 20, 25, 30, 35, 40, 50, 60, 70, 80, 90, 100, 150, 200, 250 or more). These CT547 proteins include variants (e.g. allelic variants, homologs, orthologs, paralogs, mutants, etc.) of SEQ ID NO: 10. Preferred fragments of (b) comprise an epitope from SEQ ID NO: 10. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 10. Other fragments omit one or more domains of the protein (e.g. omission of a signal peptide, of a cytoplasmic domain, of a transmembrane domain, or of an extracellular domain).

(11) Enolase (2-phosphoglycerate dehydratase) protein (CT587) One example of an ‘Eno’ protein is disclosed as SEQ ID NO^{s}: 189 & 190 in WO 03/049762 (GenBank accession number: AAC68189, GI:3329030; ‘CT587’; SEQ ID NO: 11 in attached sequence listing). Preferred Eno proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 11; and/or (b) which is a fragment of at least n consecutive amino acids of SEQ ID NO: 11, wherein n is 7 or more (e.g. 8, 10, 12, 14, 16, 18, 20, 25, 30, 35, 40, 50, 60, 70, 80, 90, 100, 150, 200, 250 or more). These Eno proteins include variants (e.g. allelic variants, homologs, orthologs, paralogs, mutants, etc.) of SEQ ID NO: 11. Preferred fragments of (b) comprise an epitope from SEQ ID NO: 11. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 11. Other fragments omit one or more domains of the protein (e.g. omission of a signal peptide, of a cytoplasmic domain, of a transmembrane domain, or of an extracellular domain). The Eno protein may contain magnesium ions, and may be in the form of a homodimer.

(12) HrtA DO protease protein (CT823) One example of an ‘HrtA’ protein is disclosed as SEQ ID NO^{s}: 229 & 230 in WO 03/049762 (GenBank accession number: AAC68420, GI:3329293; ‘CT823’; SEQ ID NO: 12 in attached sequence listing). Preferred HrtA proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 12; and/or (b) which is a fragment of at least n consecutive amino acids of SEQ ID NO: 12, wherein n is 7 or more (e.g. 8, 10, 12, 14, 16, 18, 20, 25, 30, 35, 40, 50, 60, 70, 80, 90, 100, 150, 200, 250 or more). These HrtA proteins include variants (e.g. allelic variants, homologs, orthologs, paralogs, mutants, etc.) of SEQ ID NO: 12. Preferred fragments of (b) comprise an epitope from SEQ ID NO: 12. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more; preferably at least 16 to remove the signal peptide) from the N-terminus of SEQ ID NO: 12. Other fragments omit one or more domains of the protein (e.g. omission of a signal peptide as described above, of a cytoplasmic domain, of a transmembrane domain, or of an extracellular domain). In relation to SEQ ID NO: 12, distinct domains are residues: 1-16; 17-497; 128-289; 290-381; 394-485; and 394-497.

(13) MurG peptidoglycan transferase protein (CT761) One example of a ‘MurG’ protein is disclosed as SEQ ID NO^{s}: 217 & 218 in WO 03/049762 (GenBank accession number: AAC68356, GI:3329223; ‘CT761’; SEQ ID NO: 13 in attached sequence listing). It is a UDP-N-acetylglucosamine-N-acetylmuramyl (pentapeptide) pyrophosphoryl undecaprenol-N-acetylglucosamine transferase. Preferred MurG proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 13; and/or (b) which is a fragment of at least n consecutive amino acids of SEQ ID NO: 13, wherein n is 7 or more (e.g. 8, 10, 12, 14, 16, 18, 20, 25, 30, 35, 40, 50, 60, 70, 80, 90, 100, 150, 200, 250 or more). These MurG proteins include variants (e.g. allelic variants, homologs, orthologs, paralogs, mutants, etc.) of SEQ ID NO: 13. Preferred fragments of (b) comprise an epitope from SEQ ID NO: 13. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 13. Other fragments omit one or more domains of the protein (e.g. omission of a signal peptide as described above, of a cytoplasmic domain, of a transmembrane domain, or of an extracellular domain). The MurG may be lipidated e.g. with undecaprenyl.

The immunogenicity of other known Chlamydia trachomatis antigens may be improved by combination with two or more Chlamydia trachomatis antigens from either the first antigen group or the second antigen group. Such other known Chlamydia trachomatis antigens include a third antigen group consisting of (1) PGP3, (2) one or more PMP, (3) MOMP (CT681), (4) Cap1 (CT529); (5) GroEL-like hsp60 protein (Omp2); and (6) 60 kDa Cysteine rich protein (omcB). These antigens are referred to herein as the “third antigen group”.

The invention thus includes a composition comprising a combination of Chlamydia trachomatis antigens, said combination selected from the group consisting of two, three, four, or five Chlamydia trachomatis antigens of the first antigen group and one, two, three, four, five or six Chlamydia trachomatis antigens of the third antigen group. Preferably, the combination is selected from the group consisting of three, four, or five Chlamydia trachomatis antigens from the first antigen group and three, four, or five Chlamydia trachomatis antigens from the third antigen group. Still more preferably, the combination consists of five Chlamydia trachomatis antigens from the first antigen group and three, four or five Chlamydia trachomatis antigens from the third antigen group.

The invention further includes a composition comprising a combination of Chlamydia trachomatis antigens, said combination selected from the group consisting of two, three, four, five, six, seven, eight, nine, ten, eleven, twelve or thirteen Chlamydia trachomatis antigens of the second antigen group and one, two, three, four, five or six Chlamydia trachomatis antigens of the third antigen group. Preferably, the combination is selected from the group consisting of three, four, or five Chlamydia trachomatis antigens from the second antigen group and three, four or five Chlamydia trachomatis from the third antigen group. Still more preferably, the combination consists of five Chlamydia trachomatis antigens from the second antigen group and three, four or five Chlamydia trachomatis antigens of the third antigen group.

In either of the above combinations, preferably the Chlamydia trachomatis antigens from the third antigen group include Cap 1 (CT529). Or, alternatively, in either of the above combinations, preferably the Chlamydia trachomatis antigens from the third antigen group include MOMP (CT681). Each of the Chlamydia trachomatis antigens of the third antigen group are described in more detail below.

(1) Plasmid Encoded Protein (PGP3) One example of PGP3 sequence is disclosed in, for example, at Genbank entry GI 121541. Immunization with pgp3 is discussed in Ghaem-Maghami et al., (2003) Clin. Exp. Immunol. 132: 436-442 and Donati et al., (2003) Vaccine 21:1089-1093. One example of a PGP3 protein is set forth in attached sequence listing as SEQ ID NO: 14. Preferred PGP3 proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 14; and/or (b) which is a fragment of at least n consecutive amino acids of SEQ ID NO: 14, wherein n is 7 or more (e.g. 8, 10, 12, 14, 16, 18, 20, 25, 30, 35, 40, 50, 60, 70, 80, 90, 100, 150, 200, 250 or more). These PGP3 proteins include variants (e.g. allelic variants, homologs, orthologs, paralogs, mutants, etc.) of SEQ ID NO: 14. Preferred fragments of (b) comprise an epitope from SEQ ID NO: 14. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 14. Other fragments omit one or more domains of the protein (e.g. omission of a signal peptide, of a cytoplasmic domain, of a transmembrane domain, or of an extracellular domain).

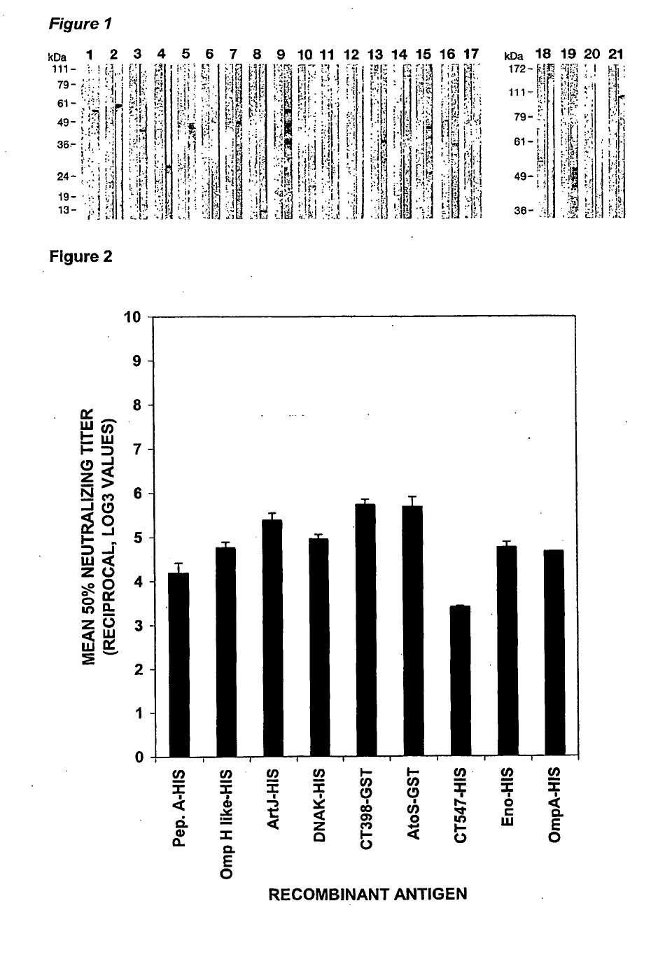

(2) Polymorphic Membrane Proteins (PMP) A family of nine Chlamydia trachomatis genes encoding predicted polymorphic membrane proteins (PMP) have been identified (pmpA to pmpI). See Stephens et al., Science (1998) 282:754-759, specifically FIG. 1. Examples of Amino acid sequences of the PMP genes are set forth as SEQ ID NOS: 15-23. (These sequences can also be found at Genbank Ref. Nos. GI 15605137 (pmpA), 15605138 (pmpB), 15605139 (pmpC), 15605546 (pmpD), 15605605 (pmpE), 15605606 (pmpF), 15605607 (pmpG), 15605608 (pmpH), and 15605610 (pmpH)). These PMP genes encode relatively large proteins (90 to 187 kDa in mass). The majority of these PMP proteins are predicted to be outer membrane proteins, and are thus also referred to as Predicted Outer Membrane Proteins. As used herein, PMP refers to one or more of the Chlamydia trachomatis pmp proteins (pmpA to pmpI) or an immunogenic fragment thereof. Preferably, the PMP protein used in the invention is pmpE or pmpI. Preferably, the PMP protein used in the invention comprises one or more of the fragments of pmpE or pmpI identified in International Patent Application PCT/US01/30345 (WO 02/28998) in Table 1 on page 20 (preferred fragments of pmpE) or Table 2 on page 21 (preferred fragments of pmpI).

Preferred PMP proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to one of the polypeptide sequences set forth as SEQ ID NOS: 15-23; and/or (b) which is a fragment of at least n consecutive amino acids of one of the polypeptide sequences set forth as SEQ ID NOS: 15-23, wherein n is 7 or more (e.g. 8, 10, 12, 14, 16, 18, 20, 25, 30, 35, 40, 50, 60, 70, 80, 90, 100, 150, 200, 250 or more). These PMP proteins include variants (e.g. allelic variants, homologs, orthologs, paralogs, mutants, etc.) of the polypeptide sequences set forth as SEQ ID NOS: 15-23. Preferred fragments of (b) comprise an epitope from one of the polypeptide sequences set forth as SEQ ID NOS: 15-23. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of one of the polypeptide sequences set forth as SEQ ID NOS: 15-23. Other fragments omit one or more domains of the protein (e.g. omission of a signal peptide, of a cytoplasmic domain, of a transmembrane domain, or of an extracellular domain).

(3) Major Outer Membrane Protein (MOMP) (CT681) One example of a MOMP sequence is disclosed as SEQ ID NOS 155 and 156 in International Patent Application No. PCT/IB02/05761 (WO 03/049762). The polypeptide sequence encoding MOMP is set forth in attached sequence listing as SEQ ID NO: 24. This protein is thought to function in vivo as a porin (See Bavoil et al, (1984) Infection and Immunity 44:479-485), and to be present during the whole life cycle of the bacteria (See Hatch et al., (1986) J. Bacteriol. 165:379-385). MOMP displays four variable domains (VD) surrounded by five constant regions that are highly conserved among serovars (See Stephens et al., (1987) J. Bacteriol. 169:3879-3885 and Yuan et al. (1989) Infection and Immunity 57: 1040-1049). In vitro and in vivo neutralizing B-cell epitopes have been mapped on VDs (See Baehr et al., (1988) PNAS USA 85:4000-4004; Lucero et al., (1985) Infection and Immunity 50:595-597; Zhang et al., (1987) J. Immunol. 138:575-581, Peterson et al., (1988) Infection and Immunity 56:885-891, Zhang et al., (1989) Infection and Immunity 57:636-638). T-cell epitopes have been identified in both variable and constant domains (See Allen et al., (1991) J. Immunol. 147:674-679 and Su et al., (1990) J. Exp. Med. 172:203-212).

Preferred MOMP proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 24; and/or (b) which is a fragment of at least n consecutive amino acids of SEQ ID NO: 24, wherein n is 7 or more (e.g. 8, 10, 12, 14, 16, 18, 20, 25, 30, 35, 40, 50, 60, 70, 80, 90, 100, 150, 200, 250 or more). These MOMP proteins include variants (e.g. allelic variants, homologs, orthologs, paralogs, mutants, etc.) of SEQ ID NO: 24. Preferred fragments of (b) comprise an epitope from SEQ ID NO: 24, preferably one or more of the B cell or T cell epitopes identified above. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 24. Other fragments omit one or more domains of the protein (e.g. omission of a signal peptide, of a cytoplasmic domain, of a transmembrane domain, or of an extracellular domain). Other preferred fragments include one or more of the conserved constant regions identified above.

(4) Cap1 (CT529 The Chlamydia trachomatis Cap1 protein corresponds with the hypothetical open reading frame CT 529 and refers to Class I Accessible Protein-1. See Fling et al., (2001) PNAS 98(3): 1160-1165. One example of a Cap1 protein is set forth herein as SEQ ID NO: 28. Predicted T-cell epitopes of Cap1 are identified in this reference as SEQ ID NO: 25 CSFIGGITYL, preferably SEQ ID NO: 26 SFIGGITYL, and SEQ ID NO: 27 SIIGGITYL.

Preferred Cap1 proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 28; and/or (b) which is a fragment of at least n consecutive amino acids of SEQ ID NO: 28, wherein n is 7 or more (e.g. 8, 10, 12, 14, 16, 18, 20, 25, 30, 35, 40, 50, 60, 70, 80, 90, 100, 150, 200, 250 or more). These Cap1 proteins include variants (e.g. allelic variants, homologs, orthologs, paralogs, mutants, etc.) of SEQ ID NO: 28. Preferred fragments of (b) comprise an epitope from SEQ ID NO: 28. Preferred T-cell epitopes include one or more of the T-cell epitopes identified above. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 28. Other fragments omit one or more domains of the protein (e.g. omission of a signal peptide, of a cytoplasmic domain, of a transmembrane domain, or of an extracellular domain).

(5) GroEL-like hsp60 protein One example of a Chlamydia trachomatis GroEL-like hsp60 protein is set forth herein as SEQ ID NO: 29. The role of Hsp60 in chlamydial infection is further described in, for example, Hessel, et al., (2001) Infection and Immunity 69(8): 4996-5000; Eckert, et al., (1997) J. Infectious Disease 175:1453-1458, Domeika et al., (1998) J. of Infectious Diseases 177:714-719; Deane et al., (1997) Clin. Exp. Immunol. 109(3): 439-445, and Peeling et al., (1997) J. Infect. Dis. 175(5):1153-1158. Immunization of guinea pig models with recombinant Hsp60 is described in Rank et al., (1995) Incest Ophthalmol. Vis. Sci. 36(7):1344-1351. B-cell epitopes of Hsp60 are identified in Yi et al., (1993) Infection & Immunity 61(3):1117-1120.

Preferred hsp60 proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 29; and/or (b) which is a fragment of at least n consecutive amino acids of SEQ ID NO: 29, wherein n is 7 or more (e.g. 8, 10, 12, 14, 16, 18, 20, 25, 30, 35, 40, 50, 60, 70, 80, 90, 100, 150, 200, 250 or more). These hsp60 proteins include variants (e.g. allelic variants, homologs, orthologs, paralogs, mutants, etc.) of SEQ ID NO: 29. Preferred fragments of (b) comprise an epitope from SEQ ID NO: 29, including one or more of the epitopes identified in the references discussed above. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 29. Other fragments omit one or more domains of the protein (e.g. omission of a signal peptide, of a cytoplasmic domain, of a .transmembrane domain, or of an extracellular domain). Other preferred fragments comprise a polypeptide sequence which does not cross-react with related human proteins.

(6) 60 kDa Cysteine rich protein (OmcB) (CT443) One example of a Chlamydia trachomatis 60 kDa Cysteine rich protein is set forth herein as SEQ ID NO: 30. This protein is also generally referred to as OmcB, Omp2 or CT 443. The role of OmcB in chlamydial infection is further described in, for example, Stephens et al., (2001) Molecular Microbiology 40(3):691-699; Millman, et al., (2001) J. of Bacteriology 183(20):5997-6008; Mygind, et al., Journal of Bacteriology (1998) 180(21):5784-5787; Bas, et al., Journal of Clinical Microbiology (2001) 39(10:4082-4085 and Goodall, et al., Clin. Exp. Immunol. (2001) 126:488-493.

Preferred OmcB proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 30; and/or (b) which is a fragment of at least n consecutive amino acids of SEQ ID NO: 30, wherein n is 7 or more (e.g. 8, 10, 12, 14, 16, 18, 20, 25, 30, 35, 40, 50, 60, 70, 80, 90, 100, 150, 200, 250 or more). These OmcB proteins include variants (e.g. allelic variants, homologs, orthologs, paralogs, mutants, etc.) of SEQ ID NO: 30. Preferred fragments of (b) comprise an epitope from SEQ ID NO: 30, including one or more of the epitopes identified in the references discussed above. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 30. Other fragments omit one or more domains of the protein (e.g. omission of a signal peptide, of a cytoplasmic domain, of a transmembrane domain, or of an extracellular domain).

The immunogenicity of other Chlamydia trachomatis antigens of known and unknown biological function may be improved by combination with two or more Chlamydia trachomatis antigens from either the first antigen group and/or the second and/or the third antigen group. Such other Chlamydia trachomatis antigens of known and unknown biological function include a fourth antigen group consisting of (1) CT559 (YscJ); (2) CT600 (Pal); (3) CT541 (Mip); (4) CT623 (CHLPN 76 kDA homologue) (5) CT700 (Hypothetical protein). (6) CT266 (Hypothetical protein); (7) CT077 (Hypothetical protein); (8) CT456 (Hypothetical protein); (9) CT165 (Hypothetical protein) and (10) CT713 (PorB). These antigens are referred to as the “fourth antigen group”.

YscJ (CT559) One example of ‘YscJ’ protein is disclosed as SEQ ID NO^{s}: 199 & 200 in WO 03/049762 (GenBank accession number: AAC68161.1 GI:3329000; ‘CT559’; SEQ ID NO: 31 in attached sequence listing). Preferred YscJ proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 31; and/or (b) which is a fragment of at least n consecutive amino acids of SEQ ID NO: 31, wherein n is 7 or more (e.g. 8, 10, 12, 14, 16, 18, 20, 25, 30, 35, 40, 50, 60, 70, 80, 90, 100, 150, 200, 250 or more). These YscJ proteins include variants (e.g. allelic variants, homologs, orthologs, paralogs, mutants, etc.) of SEQ ID NO: 31. Preferred fragments of (b) comprise an epitope from SEQ ID NO: 31. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 31. Other fragments omit one or more domains of the protein (e.g. omission of a signal peptide, of a cytoplasmic domain, of a transmembrane domain, or of an extracellular domain).

Pal (CT600) One example of a ‘Pal’ protein is disclosed as SEQ ID NO^{s}: 173 & 174 in WO 03/049762 (GenBank accession number: AAC68202.1 GI:3329044 ‘CT600’; SEQ ID NO: 32 in attached sequence listing). Preferred Pal proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 32; and/or (b) which is a fragment of at least n consecutive amino acids of SEQ ID NO 32, wherein n is 7 or more (e.g. 8, 10, 12, 14, 16, 18, 20, 25, 30, 35, 40, 50, 60, 70, 80, 90, 100, 150, 200, 250 or more). These Pal proteins include variants (e.g. allelic variants, homologs, orthologs, paralogs, mutants, etc.) of SEQ ID NO: 32. Preferred fragments of (b) comprise an epitope from SEQ ID NO: 32. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 32. Other fragments omit one or more domains of the protein (e.g. omission of a signal peptide, of a cytoplasmic domain, of a transmembrane domain, or of an extracellular domain).

Mip (CT541) One example of a ‘Mip’ protein is disclosed as SEQ ID NO^{s}: 149 & 150 in WO 03/049762 (GenBank accession number: AAC68143.1 GI:3328979 ‘CT541’; SEQ ID NO: 33 in attached sequence listing). Preferred Mip proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 33; and/or (b) which is a fragment of at least n consecutive amino acids of SEQ ID NO: 33, wherein n is 7 or more (e.g. 8, 10, 12, 14, 16, 18, 20, 25, 30, 35, 40, 50, 60, 70, 80, 90, 100, 150, 200, 250 or more). These Mip proteins include variants (e.g. allelic variants, homologs, orthologs, paralogs, mutants, etc.) of SEQ ID NO: 33. Preferred fragments of (b) comprise an epitope from SEQ ID NO: 33. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 33. Other fragments omit one or more domains of the protein (e.g. omission of a signal peptide, of a cytoplasmic domain, of a transmembrane domain, or of an extracellular domain).

CHLPN (76 kDa) (CT623) One example of a CHLPN (76 kDa protein) is disclosed as SEQ ID NO^{s}: 163 & 164 in WO 03/049762 (GenBank accession number: AAC68227.2 GI:6578109 ‘CT623’; SEQ ID NO: 34 in the attached sequence listing). Preferred CHLPN (76 kDa protein proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 34; and/or (b) which is a fragment of at least n consecutive amino acids of SEQ ID NO: 34, wherein n is 7 or more (e.g. 8, 10, 12, 14, 16, 18, 20, 25, 30, 35, 40, 50, 60, 70, 80, 90, 100, 150, 200, 250 or more). These CHLPN (76 kDa protein) proteins include variants (e.g. allelic variants, homologs, orthologs, paralogs, mutants, etc.) of SEQ ID NO: 34. Preferred fragments of (b) comprise an epitope from SEQ ID NO: 34. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 34. Other fragments omit one or more domains of the protein (e.g. omission of a signal peptide, of a cytoplasmic domain, of a transmembrane domain, or of an extracellular domain).

Hypothetical Protein (CT700) One example of a CT700 Hypothetical Protein is disclosed as SEQ ID NO^{s} 261 & 262 in WO 03/049762 (GenBank accession number: AAC68295.1 GI:3329154 ‘CT700’; SEQ ID NO: 35 in attached sequence listing). Preferred CT700 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 35; and/or (b) which is a fragment of at least n consecutive amino acids of SEQ ID NO: 35, wherein n is 7 or more (e.g. 8, 10, 12, 14, 16, 18, 20, 25, 30, 35, 40, 50, 60, 70, 80, 90, 100, 150, 200, 250 or more). These CT700 Hypothetical proteins include variants (e.g. allelic variants, homologs, orthologs, paralogs, mutants, etc.) of SEQ ID NO: 35. Preferred fragments of (b) comprise an epitope from SEQ ID NO: 35. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 35. Other fragments omit one or more domains of the protein (e.g. omission of a signal peptide, of a cytoplasmic domain, of a transmembrane domain, or of an extracellular domain).

Hypothetical Protein (CT 266) One example of a CT266 Hypothetical Protein is disclosed as SEQ ID NO^{s} 77 & 78 in WO 03/049762 (GenBank accession number AAC67859.1 GI:3328678 ‘CT266’; SEQ ID NO: 36 in attached sequence listing). Preferred CT266 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 36; and/or (b) which is a fragment of at least n consecutive amino acids of SEQ ID NO: 36, wherein n is 7 or more (e.g. 8, 10, 12, 14, 16, 18, 20, 25, 30, 35, 40, 50, 60, 70, 80, 90, 100, 150, 200, 250 or more). These CT266 Hypothetical proteins include variants (e.g. allelic variants, homologs, orthologs, paralogs, mutants, etc.) of SEQ ID NO: 36. Preferred fragments of (b) comprise an epitope from SEQ ID NO: 36. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 36. Other fragments omit one or more domains of the protein (e.g. omission of a signal peptide, of a cytoplasmic domain, of a transmembrane domain, or of an extracellular domain).

Hypothetical Protein (CT077) One example of a CT077 Hypothetical Protein is disclosed as SEQ ID NO^{s} 65 & 66 in WO 03/049762 (GenBank accession number: AAC67668.1 GI:3328472 ‘CT077’; SEQ ID NO: 37 in attached sequence listing). Preferred CT077 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 37; and/or (b) which is a fragment of at least n consecutive amino acids of SEQ ID NO: 37, wherein n is 7 or more (e.g. 8, 10, 12, 14, 16, 18, 20, 25, 30, 35, 40, 50, 60, 70, 80, 90, 100, 150, 200, 250 or more). These CT077 Hypothetical proteins include variants (e.g. allelic variants, homologs, orthologs, paralogs, mutants; etc.) of SEQ ID NO: 37. Preferred fragments of (b) comprise an epitope from SEQ ID NO: 37. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 37. Other fragments omit one or more domains of the protein (e.g. omission of a signal peptide, of a cytoplasmic domain, of a transmembrane domain, or of an extracellular domain).

Hypothetical Protein (CT456) One example of a CT456 Hypothetical Protein is disclosed as SEQ ID NO^{s} 255 & 256 in WO 03/049762 (GenBank accession number: AAC68056.1 GI:3328889 ‘CT456’; SEQ ID NO: 38 in attached sequence listing). Preferred CT456 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 38; and/or (b) which is a fragment of at least n consecutive amino acids of SEQ ID NO: 38, wherein n is 7 or more (e.g. 8, 10, 12, 14, 16, 18, 20, 25, 30, 35, 40, 50, 60, 70, 80, 90, 100, 150, 200, 250 or more). These CT456 Hypothetical proteins include variants (e.g. allelic variants, homologs, orthologs, paralogs, mutants, etc.) of SEQ ID NO: 38. Preferred fragments of (b) comprise an epitope from SEQ ID NO: 38. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 38. Other fragments omit one or more domains of the protein (e.g. omission of a signal peptide, of a cytoplasmic domain, of a transmembrane domain, or of an extracellular domain).

Hypothetical Protein (CT165) One example of a CT165 Hypothetical Protein is disclosed (GenBank accession number: AAC67756.1 GI:3328568 CT165’; SEQ ID NO: 39 in attached sequence listing). Preferred Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 39; and/or (b) which is a fragment of at least n consecutive amino acids of SEQ ID NO: 39, wherein n is 7 or more (e.g. 8, 10, 12, 14, 16, 18, 20, 25, 30, 35, 40, 50, 60, 70, 80, 90, 100, 150, 200, 250 or more). These CT165 Hypothetical proteins include variants (e.g. allelic variants, homologs, orthologs, paralogs, mutants, etc.) of SEQ ID NO: 39. Preferred fragments of (b) comprise an epitope from SEQ ID NO: 39. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 39. Other fragments omit one or more domains of the protein (e.g. omission of a signal peptide, of a cytoplasmic domain, of a transmembrane domain, or of an extracellular domain).

PorB (CT713) One example of a PorB Protein is disclosed as SEQ ID NO^{s} 201 & 202 in WO 03/049762 (GenBank accession number: AAC68308.1 GI:3329169 ‘CT713’; SEQ ID NO: 40 in attached sequence listing). Preferred PorB proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 40; and/or (b) which is a fragment of at least n consecutive amino acids of SEQ ID NO: 40, wherein n is 7 or more (e.g. 8, 10, 12, 14, 16, 18, 20, 25, 30, 35, 40, 50, 60, 70, 80, 90, 100, 150, 200, 250 or more). These PorB proteins include variants (e.g. allelic variants, homologs, orthologs, paralogs, mutants, etc.) of SEQ ID NO: 40. Preferred fragments of (b) comprise an epitope from SEQ ID NO: 40. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 40. Other fragments omit one or more domains of the protein (e.g. omission of a signal peptide, of a cytoplasmic domain, of a transmembrane domain, or of an extracellular domain).

The immunogenicity of other Chlamydia trachomatis antigens of known and unknown biological function may be improved by combination with two or more Chlamydia trachomatis antigens from either the first antigen group and/or the second and/or the third antigen group and/or the fourth antigen group. Such other Chlamydia trachomatis antigens of known and unknown biological function include a fifth antigen group consisting of: (1) CT082 (hypothetical); (2) CT181 (Hypothetical); (3) CT050 (Hypothetical); (4) CT157 (Phospholipase D superfamily); and (5) CT128 (AdK adenylate cyclase).

Hypothetical Protein (CT082) One example of a CT082 Hypothetical Protein is disclosed as (GenBank accession number: AAC67673.1 GI:3328477 ‘CT082’; SEQ ID NO: 41 in attached sequence listing). Preferred CT082 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 41; and/or (b) which is a fragment of at least n consecutive amino acids of SEQ ID NO: 41, wherein n is 7 or more (e.g. 8, 10, 12, 14, 16, 18, 20, 25, 30, 35, 40, 50, 60, 70, 80, 90, 100, 150, 200, 250 or more). These CT082 Hypothetical proteins include variants (e.g. allelic variants, homologs, orthologs, paralogs, mutants, etc.) of SEQ ID NO: 41. Preferred fragments of (b) comprise an epitope from SEQ ID NO: 41. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 41. Other fragments omit one or more domains of the protein (e.g. omission of a signal peptide, of a cytoplasmic domain, of a transmembrane domain, or of an extracellular domain).

Hypothetical Protein (CT181) One example of a CT181 Hypothetical Protein is disclosed as SEQ ID NO^{s} 245 & 246 in WO 03/049762 (GenBank accession number: AAC67772.1 GI:3328585 ‘CT181’; SEQ ID NO: 42 in attached sequence listing). Preferred CT181 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 42; and/or (b) which is a fragment of at least n consecutive amino acids of SEQ ID NO: 42, wherein n is 7 or more (e.g. 8, 10, 12, 14, 16, 18, 20, 25, 30, 35, 40, 50, 60, 70, 80, 90, 100, 150, 200, 250 or more). These CT181 Hypothetical proteins include variants (e.g. allelic variants, homologs, orthologs, paralogs, mutants, etc.) of SEQ ID NO: 42. Preferred fragments of (b) comprise an epitope from SEQ ID NO: 42. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 42. Other fragments omit one or more domains of the protein (e.g. omission of a signal peptide, of a cytoplasmic domain, of a transmembrane domain, or of an extracellular domain).

Hypothetical Protein (CT050) One example of a CT050 Hypothetical Protein is disclosed as (GenBank accession number: AAC67641.1 GI:3328442 ‘CT050’; SEQ ID NO: 43 in attached sequence listing). Preferred CT050 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 43; and/or (b) which is a fragment of at least n consecutive amino acids of SEQ ID NO: 43, wherein n is 7 or more (e.g. 8, 10, 12, 14, 16, 18, 20, 25, 30, 35, 40, 50, 60, 70, 80, 90, 100, 150, 200, 250 or more). These CT050 Hypothetical proteins include variants (e.g. allelic variants, homologs, orthologs, paralogs, mutants, etc.) of SEQ ID NO: 43. Preferred fragments of (b) comprise an epitope from SEQ ID NO: 43. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 43. Other fragments omit one or more domains of the protein (e.g. omission of a signal peptide, of a cytoplasmic domain, of a transmembrane domain, or of an extracellular domain).

Phospholipase D SuperFamily (CT157) One example of a Phospholipase D SuperFamily Protein is disclosed as (GenBank accession number: AAC67748.1 G1:3328559 ‘CT157’; SEQ ID NO: 44 in attached sequence listing). Preferred Phospholipase D SuperFamily proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 44; and/or (b) which is a fragment of at least n consecutive amino acids of SEQ ID NO: 44, wherein n is 7 or more (e.g. 8, 10, 12, 14, 16, 18, 20, 25, 30, 35, 40, 50, 60, 70, 80, 90, 100, 150, 200, 250 or more). These Phospholipase D SuperFamily proteins include variants (e.g. allelic variants, homologs, orthologs, paralogs, mutants, etc.) of SEQ ID NO: 44. Preferred fragments of (b) comprise an epitope from SEQ ID NO: 44. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 44. Other fragments omit one or more domains of the protein (e.g. omission of a signal peptide, of a cytoplasmic domain, of a transmembrane domain, or of an extracellular domain).

AdK (Adenylate Kinase) (CT128) One example of an Adenylate Kinase Protein is disclosed as (GenBank accession number: AAC67719.1 GI:3328527 ‘CT128’; SEQ ID NO: 45 in attached sequence listing). Preferred Adenylate Kinase proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 45; and/or (b) which is a fragment of at least n consecutive amino acids of SEQ ID NO: 45, wherein n is 7 or more (e.g. 8, 10, 12, 14, 16, 18, 20, 25, 30, 35, 40, 50, 60, 70, 80, 90, 100, 150, 200, 250 or more). These Adenylate Kinase proteins include variants (e.g. allelic variants, homologs, orthologs, paralogs, mutants, etc.) of SEQ ID NO: 45. Preferred fragments of (b) comprise an epitope from SEQ ID NO: 45. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 45. Other fragments omit one or more domains of the protein (e.g. omission of a signal peptide, of a cytoplasmic domain, of a transmembrane domain, or of an extracellular domain).

The immunogenicity of other Chlamydia trachomatis antigens of known and unknown biological function may be improved by combination with two or more Chlamydia trachomatis antigens from either the first antigen group and/or the second and/or the third antigen group and/or the fourth antigen group and/or the fifth antigen group. Such other Chlamydia trachomatis antigens of known and unknown biological function include a sixth antigen group consisting of: (1) CT153 (Hypothetical); (2) CT262 (Hypothetical); (3) CT276 (Hypothetical); (4) CT296 (Hypothetical); (5) CT372 (Hypothetical); (6) CT412 (PmpA); (7) CT480 (OligoPeptide Binding Protein); (8) CT548 (Hypothetical); (9) CT043 (Hypothetical); (10) CT635 (Hypothetical); (11) CT859 (Metalloprotease); (12) CT671 (Hypothetical); (13) CT016 (Hypothetical); (14) CT017 (Hypothetical); (15) CT043 (Hypothetical); (16) CT082 (Hypothetical); (17) CT548 (Hypothetical); (19) CT089 (Low Calcium Response Element); (20) CT812 (PmpD) and (21) CT869 (PmpE).

Hypothetical Protein (CT153) One example of a CT153 Hypothetical Protein is disclosed as (GenBank accession number: AAC67744.1 GI:3328555 ‘CT153’; SEQ ID NO: 46 in attached sequence listing). Preferred CT153 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 46; and/or (b) which is a fragment of at least n consecutive amino acids of SEQ ID NO: 46, wherein n is 7 or more (e.g. 8, 10, 12, 14, 16, 18, 20, 25, 30, 35, 40, 50, 60, 70, 80, 90, 100, 150, 200, 250 or more). These CT153 Hypothetical proteins include variants (e.g. allelic variants, homologs, orthologs, paralogs, mutants, etc.) of SEQ ID NO: 46. Preferred fragments of (b) comprise an epitope from SEQ ID NO: 46. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 46. Other fragments omit one or more domains of the protein (e.g. omission of a signal peptide, of a cytoplasmic domain, of a transmembrane domain, or of an extracellular domain).

Hypothetical Protein (CT262) One example of a CT262 Hypothetical Protein is disclosed as (GenBank accession number: AAC67835.1 GI:3328652 ‘CT262’; SEQ ID NO: 47 in attached sequence listing). Preferred CT262 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 47; and/or (b) which is a fragment of at least n consecutive amino acids of SEQ ID NO: 47, wherein n is 7 or more (e.g. 8, 10, 12, 14, 16, 18, 20, 25, 30, 35, 40, 50, 60, 70, 80, 90, 100, 150, 200, 250 or more). These CT262 Hypothetical proteins include variants (e.g. allelic variants, homologs, orthologs, paralogs, mutants, etc.) of SEQ ID NO: 47. Preferred fragments of (b) comprise an epitope from SEQ ID NO: 47. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 47. Other fragments omit one or more domains of the protein (e.g. omission of a signal peptide, of a cytoplasmic domain, of a transmembrane domain, or of an extracellular domain).

Hypothetical Protein (CT276) One example of a CT276 Hypothetical Protein is disclosed as (GenBank accession number: AAC67869.1 GI:3328689 ‘CT276’; SEQ ID NO: 48 in attached sequence listing). Preferred CT276 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 48; and/or (b) which is a fragment of at least n consecutive amino acids of SEQ ID NO: 48, wherein n is 7 or more (e.g. 8, 10, 12, 14, 16, 18, 20, 25, 30, 35, 40, 50, 60, 70, 80, 90, 100, 150, 200, 250 or more). These CT276 Hypothetical proteins include variants (e.g. allelic variants, homologs, orthologs, paralogs, mutants, etc.) of SEQ ID NO: 48. Preferred fragments of (b) comprise an epitope from SEQ ID NO: 48. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 48. Other fragments omit one or more domains of the protein (e.g. omission of a signal peptide, of a cytoplasmic domain, of a transmembrane domain, or of an extracellular domain).

Hypothetical Protein (CT296) One example of a CT296 Hypothetical Protein is disclosed as (GenBank accession number: AAC67889.1 GI:3328711 ‘CT296’; SEQ ID NO: 49 in attached sequence listing). Preferred CT296 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 49; and/or (b) which is a fragment of at least n consecutive amino acids of SEQ ID NO: 49, wherein n is 7 or more (e.g. 8, 10, 12, 14, 16, 18, 20, 25, 30, 35, 40, 50, 60, 70, 80, 90, 100, 150, 200, 250 or more). These CT296 Hypothetical proteins include variants (e.g. allelic variants, homologs, orthologs, paralogs, mutants, etc.) of SEQ ID NO: 49. Preferred fragments of (b) comprise an epitope from SEQ ID NO: 49. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 49. Other fragments omit one or more domains of the protein (e.g. omission of a signal peptide, of a cytoplasmic domain, of a transmembrane domain, or of an extracellular domain).

Hypothetical Protein (CT372) One example of a CT372 Hypothetical Protein is disclosed as SEQ ID NO^{s} 187 & 188 in WO 03/049762 (GenBank accession number: AAC67968.1 GI:3328796 ‘CT372’; SEQ ID NO: 50 in attached sequence listing). Preferred CT372 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 50; and/or (b) which is a fragment of at least n consecutive amino acids of SEQ ID NO: 50, wherein n is 7 or more (e.g. 8, 10, 12, 14, 16, 18, 20, 25, 30, 35, 40, 50, 60, 70, 80, 90, 100, 150, 200, 250 or more). These CT372 Hypothetical proteins include variants (e.g. allelic variants, homologs, orthologs, paralogs, mutants, etc.) of SEQ ID NO: 50. Preferred fragments of (b) comprise an epitope from SEQ ID NO: 50. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 50. Other fragments omit one or more domains of the protein (e.g. omission of a signal peptide, of a cytoplasmic domain, of a transmembrane domain, or of an extracellular domain).

Putative Outer Membrane Protein A (PmpA) (CT412) One example of a PmpA Protein is disclosed as SEQ ID NO^{s} 89 & 90 in WO 03/049762 (GenBank accession number: AAC68009.1 GI:3328840 ‘CT412’; SEQ ID NO: 51 in attached sequence listing and also SEQ ID No 15 above). Preferred PmpA proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 51; and/or (b) which is a fragment of at least n consecutive amino acids of SEQ ID NO: 51, wherein n is 7 or more (e.g. 8, 10, 12, 14, 16, 18, 20, 25, 30, 35, 40, 50, 60, 70, 80, 90, 100, 150, 200, 250 or more). These PmpA proteins include variants (e.g. allelic variants, homologs, orthologs, paralogs, mutants, etc.) of SEQ ID NO: 51. Preferred fragments of (b) comprise an epitope from SEQ ID NO: 51. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 51. Other fragments omit one or more domains of the protein (e.g. omission of a signal peptide, of a cytoplasmic domain, of a transmembrane domain, or of an extracellular domain).

Oligopeptide Binding Lipoprotein (CT480) One example of an OligoPeptide Binding Protein is disclosed as SEQ ID NO^{s} 141 & 142 in WO 03/049762 (GenBank accession number: AAC68080.1 GI:3328915 ‘CT480’; SEQ ID NO: 52 in attached sequence listing). Preferred OligoPeptide Binding proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 52; and/or (b) which is a fragment of at least n consecutive amino acids of SEQ ID NO: 52, wherein n is 7 or more (e.g. 8, 10, 12, 14, 16, 18, 20, 25, 30, 35, 40, 50, 60, 70, 80, 90, 100, 150, 200, 250 or more). These OligoPeptide Binding proteins include variants (e.g. allelic variants, homologs, orthologs, paralogs, mutants, etc.) of SEQ ID NO: 52. Preferred fragments of (b) comprise an epitope from SEQ ID NO: 52. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 52. Other fragments omit one or more domains of the protein (e.g. omission of a signal peptide, of a cytoplasmic domain, of a transmembrane domain, or of an extracellular domain).

Hypothetical Protein (CT548) One example of a Hypothetical Protein is disclosed as SEQ ID NO^{s} 153 & 154 in WO 03/049762 (GenBank accession number: AAC68150.1 GI:3328987 ‘CT548’; SEQ ID NO: 53 in attached sequence listing). Preferred CT548 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 53; and/or (b) which is a fragment of at least n consecutive amino acids of SEQ ID NO: 53, wherein n is 7 or more (e.g. 8, 10, 12, 14, 16, 18, 20, 25, 30, 35, 40, 50, 60, 70, 80, 90, 100, 150, 200, 250 or more). These CT548 Hypothetical proteins include variants (e.g. allelic variants, homologs, orthologs, paralogs, mutants, etc.) of SEQ ID NO: 53. Preferred fragments of (b) comprise an epitope from SEQ ID NO: 53. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 53. Other fragments omit one or more domains of the protein (e.g. omission of a signal peptide, of a cytoplasmic domain, of a transmembrane domain, or of an extracellular domain).

Hypothetical Protein (CT043) One example of a CT043 Hypothetical Protein is disclosed as (GenBank accession number: AAC67634.1 GI:3328435 ‘CT043’; SEQ ID NO: 54 in attached sequence listing). Preferred CT043 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5%. or more) to SEQ ID NO: 54; and/or (b) which is a fragment of at least n consecutive amino acids of SEQ ID NO: 54, wherein n is 7 or more (e.g. 8, 10, 12, 14, 16, 18, 20, 25, 30, 35, 40, 50, 60, 70, 80, 90, 100, 150, 200, 250 or more). These CT043 Hypothetical proteins include variants (e.g. allelic variants, homologs, orthologs, paralogs, mutants, etc.) of SEQ ID NO: 54. Preferred fragments of (b) comprise an epitope from SEQ ID NO: 54. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 54. Other fragments omit one or more domains of the protein (e.g. omission of a signal peptide, of a cytoplasmic domain, of a transmembrane domain, or of an extracellular domain).

Hypothetical Protein (CT635) One example of a CT635 Hypothetical Protein is disclosed as (GenBank accession number: AAC68239.1 GI:3329083 ‘CT635’; SEQ ID NO: 55 in attached sequence listing). Preferred CT635 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 55; and/or (b) which is a fragment of at least n consecutive amino acids of SEQ ID NO: 55, wherein n is 7 or more (e.g. 8, 10, 12, 14, 16, 18, 20, 25, 30, 35, 40, 50, 60, 70, 80, 90, 100, 150, 200, 250 or more). These CT635 Hypothetical proteins include variants (e.g. allelic variants, homologs, orthologs, paralogs, mutants, etc.) of SEQ ID NO: 55. Preferred fragments of (b) comprise an epitope from SEQ ID NO: 55. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 55. Other fragments omit one or more domains of the protein (e.g. omission of a signal peptide, of a cytoplasmic domain, of a transmembrane domain, or of an extracellular domain).

Metalloprotease (CT859) One example of a Metalloproease Protein is disclosed as (GenBank accession number: ‘CT859’ AAC68457.1 GI:3329333; SEQ ID NO: 56 in attached sequence listing). Preferred Metalloprotease proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 56; and/or (b) which is a fragment of at least n consecutive amino acids of SEQ ID NO: 56, wherein n is 7 or more (e.g. 8, 10, 12, 14, 16, 18, 20, 25, 30, 35, 40, 50, 60, 70, 80, 90, 100, 150, 200, 250 or more). These Metalloprotease proteins include variants (e.g. allelic variants, homologs, orthologs, paralogs, mutants, etc.) of SEQ ID NO: 56. Preferred fragments of (b) comprise an epitope from SEQ ID NO: 56. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 56. Other fragments omit one or more domains of the protein (e.g. omission of a signal peptide, of a cytoplasmic domain, of a transmembrane domain, or of an extracellular domain).

Hypothetical Protein (CT671) One example of a CT671 Hypothetical Protein is disclosed as (GenBank accession number: AAC68266.1 GI:3329122 ‘CT671’; SEQ ID NO: 57 in attached sequence listing). Preferred CT671 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 57; and/or (b) which is a fragment of at least n consecutive amino acids of SEQ ID NO: 57, wherein n is 7 or more (e.g. 8, 10, 12, 14, 16, 18, 20, 25, 30, 35, 40, 50, 60, 70, 80, 90, 100, 150, 200, 250 or more). These CT671 Hypothetical proteins include variants (e.g. allelic variants, homologs, orthologs, paralogs, mutants, etc.) of SEQ ID NO: 57. Preferred fragments of (b) comprise an epitope from SEQ ID NO: 57. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 57. Other fragments omit one or more domains of the protein (e.g. omission of a signal peptide, of a cytoplasmic domain, of a transmembrane domain, or of an extracellular domain).

Hypothetical Protein (CT016) One example of a CT016 Hypothetical Protein is disclosed as (GenBank accession number: AAC67606.1 GI:3328405 ‘CT016’; SEQ ID NO: 58 in attached sequence listing). Preferred CT016 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 58; and/or (b) which is a fragment of at least n consecutive amino acids of SEQ ID NO: 58, wherein n is 7 or more (e.g. 8, 10, 12, 14, 16, 18, 20, 25, 30, 35, 40, 50, 60, 70, 80, 90, 100, 150, 200, 250 or more). These CT016 Hypothetical proteins include variants (e.g. allelic variants, homologs, orthologs, paralogs, mutants, etc.) of SEQ ID NO: 58. Preferred fragments of (b) comprise an epitope from SEQ ID NO: 58. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 58. Other fragments omit one or more domains of the protein (e.g. omission of a signal peptide, of a cytoplasmic domain, of a transmembrane domain, or of an extracellular domain).

Hypothetical Protein (CT017) One example of a CT017 Hypothetical Protein is disclosed as (GenBank accession number: AAC67607.1 GI:3328406 ‘CT017’; SEQ ID NO: 59 in attached sequence listing). Preferred CT017 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 59; and/or (b) which is a fragment of at least n consecutive amino acids of SEQ ID NO: 59, wherein n is 7 or more (e.g. 8, 10, 12, 14, 16, 18, 20, 25, 30, 35, 40, 50, 60, 70, 80, 90, 100, 150, 200, 250 or more). These CT017 Hypothetical proteins include variants (e.g. allelic variants, homologs, orthologs, paralogs, mutants, etc.) of SEQ ID NO: 59. Preferred fragments of (b) comprise an epitope from SEQ ID NO: 59. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 59. Other fragments omit one or more domains of the protein (e.g. omission of a signal peptide, of a cytoplasmic domain, of a transmembrane domain, or of an extracellular domain).

Hypothetical Protein (CT043) One example of a CT043 Hypothetical Protein is disclosed as (GenBank accession number: AAC67634.1 GI:3328435 ‘CT043’; SEQ ID NO: 60 in attached sequence listing). Preferred CT043 Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 60; and/or (b) which is a fragment of at least n consecutive amino acids of SEQ ID NO: 60, wherein n is 7 or more (e.g. 8, 10, 12, 14, 16, 18, 20, 25, 30, 35, 40, 50, 60, 70, 80, 90, 100, 150, 200, 250 or more). These CT043 Hypothetical proteins include variants (e.g. allelic variants, homologs, orthologs, paralogs, mutants, etc.) of SEQ ID NO: 60. Preferred fragments of (b) comprise an epitope from SEQ ID NO: 60. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 60. Other fragments omit one or more domains of the protein (e.g. omission of a signal peptide, of a cytoplasmic domain, of a transmembrane domain, or of an extracellular domain).

Hypothetical Protein (CT082) This hypothetical protein is already discussed above as SEQ ID No 39.

Hypothetical Protein (CT548) One example of a Hypothetical Protein is disclosed as (GenBank accession number: AAC68150.1 GI:3328987 ‘CT548’; SEQ ID NO: 61 in attached sequence listing). Preferred Hypothetical proteins for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%;99%, 99.5% or more) to SEQ ID NO: 61; and/or (b) which is a fragment of at least n consecutive amino acids of SEQ ID NO: 61, wherein n is 7 or more (e.g. 8, 10, 12, 14, 16, 18, 20, 25, 30, 35, 40, 50, 60, 70, 80, 90, 100, 150, 200, 250 or more). These Hypothetical proteins include variants (e.g. allelic variants, homologs, orthologs, paralogs, mutants, etc.) of SEQ ID NO: 61. Preferred fragments of (b) comprise an epitope from SEQ ID NO: 61. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 61. Other fragments omit one or more domains of the protein (e.g. omission of a signal peptide, of a cytoplasmic domain, of a transmembrane domain, or of an extracellular domain).

LcrE (CT089) This Low Calcium Response Element protein is discussed above as SEQ ID NO: 2 and SEQ ID NO 41.

PmpD (CT812) This polymorphic membrane protein D is discussed above as SEQ ID NO: 18 (CT812).

PmpE (CT869) This polymorphic membrane protein E is discussed above as SEQ ID NO: 19.

The invention includes a composition comprising a combination of Chlamydia trachomatis antigens, said combination selected from the group consisting of two, three, four, or five Chlamydia trachomatis antigens of the first antigen group and one, two, three, four, or five antigens of the fourth antigen group.

The invention includes a composition comprising a combination of Chlamydia trachomatis antigens, said combination selected from the group consisting of two, three, four, or five Chlamydia trachomatis antigens of the first antigen group and one, two, three, four or five antigens of the fifth antigen group.

The invention includes a composition comprising a combination of Chlamydia trachomatis antigens, said combination selected from the group consisting of two, three, four, or five Chlamydia trachomatis antigens of the first antigen group and one, two, three, four or five antigens of the sixth antigen group.

The invention includes a composition comprising a combination of Chlamydia trachomatis antigens, said combination selected from the group consisting of two, three, four, or five Chlamydia trachomatis antigens of the second antigen group and one, two, three, four or five antigens of the fourth antigen group.

The invention includes a composition comprising a combination of Chlamydia trachomatis antigens, said combination selected from the group consisting of two, three, four, or five Chlamydia trachomatis antigens of the second antigen group and one, two, three, four or five antigens of the fifth antigen group.

The invention includes a composition comprising a combination of Chlamydia trachomatis antigens, said combination selected from the group consisting of two, three, four, or five Chlamydia trachomatis antigens of the second antigen group and one, two, three, four or five antigens of the sixth antigen group.

The invention thus includes a composition comprising a combination of Chlamydia trachomatis antigens, said combination selected from the group consisting of two, three, four, or five Chlamydia trachomatis antigens of the first antigen group and one, two, three, four, five or six Chlamydia trachomatis antigens of the third antigen group and one, two, three, four, five, six, seven, eight, nine or ten antigens of the fourth antigen group and one, two, three, four or five Chlamydia trachomatis antigens of the fifth antigen group and one, two, three, four, five, six, seven, eight, nine, ten, eleven or twelve antigens of the sixth antigen group.

Preferably, the combination is selected from the group consisting of three, four, or five Chlamydia trachomatis antigens from the first antigen group and three, four, or five Chlamydia trachomatis antigens from the third antigen group and three, four or five Chlamydia trachomatis antigens from the fourth antigen group and one; two, three, four or five Chlamydia trachomatis antigens of the fifth antigen group and one, two, three, four, five, six, seven, eight, nine, ten, eleven or twelve antigens of the sixth antigen group.

Still more preferably, the combination consists of five Chlamydia trachomatis antigens from the first antigen group and three, four or five Chlamydia trachomatis antigens from the third antigen group and three, four or five antigens from the fourth antigen group and one, two, three, four, five or six Chlamydia trachomatis antigens of the fifth antigen group and one, two, three, four, five, six, seven, eight, nine, ten, eleven or twelve antigens of the sixth antigen group.

The invention further includes a composition comprising a combination of Chlamydia trachomatis antigens, said combination selected from the group consisting of two, three, four, five, six, seven, eight, nine, ten, eleven, twelve or thirteen Chlamydia trachomatis antigens of the second antigen group and one, two, three, four, five or six Chlamydia trachomatis antigens of the third antigen group and one, two, three, four, five, six, seven, eight or nine antigens of the fourth antigen group. Preferably, the combination is selected from the group consisting of three, four, or five Chlamydia trachomatis antigens from the second antigen group and three, four or five Chlamydia trachomatis from the third antigen group and three, four or five antigens of the fourth antigen group. Still more preferably, the combination consists of five Chlamydia trachomatis antigens from the second antigen group and three, four or five Chlamydia trachomatis antigens of the third antigen group and three, four or five antigens of the fourth antigen group.

There is an upper limit to the number of Chlamydia trachomatis antigens which will be in the compositions of the invention. Preferably, the number of Chlamydia trachomatis antigens in a composition of the invention is less than 20, less than 19, less than 18, less than 17, less than 16, less than 15, less than 14, less than 13, less than 12, less than 11, less than 10, less than 9, less than 8, less than 7, less than 6, less than 5, less than 4, or less than 3. Still more preferably, the number of Chlamydia trachomatis antigens in a composition of the invention is less than 6, less than 5, or less than 4. The Chlamydia trachomatis antigens used in the invention are preferably isolated, i.e., separate and discrete, from the whole organism with which the molecule is found in nature or, when the polynucleotide or polypeptide is not found in nature, is sufficiently free of other biological macromolecules so that the polynucleotide or polypeptide can be used for its intended purpose.

Preferably, the composition of the present invention comprises a combination of Chlamydia trachomatis antigens, wherein said combination selected from the group consisting of: (1) CT016 and CT128 and CT671 and CT262; (2) CT296 and CT372 and CT635 and CT859; (3) CT412 and CT480 and CT869 and CT871; (4) CT050 and CT153 and CT157 and CT165; (5) CT276 and CT296 and CT456 and CT480; (6) CT089 and CT381 and CT396 and CT548; (7) CT635 and CT700 and CT711 and CT859; (8) CT812 and CT869 and CT552 and CT671; (9) CT713 and CT017 and CT043 and CT082; (10) CT266 and CT443 and CT559 and CT597; and (11) CT045 and CT089 and CT396 and CT398 and CT39 (12) CT681 and CT547; (13) CT623 and CT414; or other combinations thereof.

Preferably, the composition of the present invention comprises a combination of Chlamydia trachomatis antigens, said combination selected from the group consisting of: (1) CT016 and CT128 and CT671 and CT262; (2) CT296 and CT372 and CT635 and CT859; (3) CT412 and CT480 and CT869 and CT871; (4) CT050 and CT153 and CT157 and CT165; (5) CT276 and CT296 and CT456 and CT480; (6) CT089 and CT381 and CT396 and CT548; (7) CT635 and CT700 and CT711 and CT859; (8) CT812 and CT869 and CT552 and CT671; (9) CT713 and CT017 and CT043 and CT082; (10) CT266 and CT443 and CT559 and CT597; and (11) CT045 and CT089 and CT396 and CT398 and CT39 (12) CT681 and CT547; (13) CT623 and CT414; or other combinations thereof; in combination with an immunoregulatory agent which is selected from the group consisting of CFA, Alum, CpG, AlOH, Alum and CpG, AlOH and CpG, LTK63 and LTK63 and CpG.

Preferably, the composition of the present invention comprises a combination of Chlamydia trachomatis antigens, said combination selected from the group consisting of: 1) CT016 and CT128 and CT671 and CT262; (2) CT296 and CT372 and CT635 and CT859; (3) CT412 and CT480 and CT869 and CT871; (4) CT050 and CT153 and CT157 and CT165; (5) CT276 and CT296 and CT456 and CT480; (6) CT089 and CT381 and CT396 and CT548; (7) CT635 and CT700 and CT711 and CT859; (8) CT812 and CT869 and CT552 and CT671; (9) CT713 and CT017 and CT043 and CT082; (10) CT266 and CT443 and CT559 and CT597; and (11) CT045 and CT089 and CT396 and CT398 and CT39 (12) CT681 and CT547; (13) CT623 and CT414; or other combinations thereof; in combination with Alum and CpG or AlOH and CpG.

Preferably, the composition of the present invention comprises a combination of Chlamydia trachomatis antigens, said combination selected from the group consisting of (1) CT242 and CT316; (2) CT467 and CT444; and (3) CT812 and CT082; or other combinations thereof.

Preferably, the composition of the present invention comprises a combination of Chlamydia trachomatis antigens, said combination selected from the group consisting of (1) CT242 and CT316; (2) CT467 and CT444; and (3) CT812 and CT082; or other combinations thereof in combination with an immunoregulatory agent which is selected from the group consisting of CFA, Alum, CpG, AlOH, Alum and CpG, AlOH and CpG, LTK63 and LTK63 and CpG.

Preferably, the composition of the present invention comprises a combination of Chlamydia trachomatis antigens, said combination selected from the group consisting of (1) CT242 and CT316; (2) CT467 and CT444; and (3) CT812 and CT082; or other combinations thereof in combination with Alum and CpG or AlOH and CpG.

The immunogenic compositions of the present invention may comprise one or more antigens selected from a “fourth antigen” group consisting of: (1) CT559 (YscJ); (2) CT600 (Pal); (3) CT541 (Mip); (4) CT623 (CHLPN 76 kDA homologue) (5) CT700 (Hypothetical protein). (6) CT266 (Hypothetical protein); (7) CT077 (Hypothetical protein); (8) CT456 (Hypothetical protein); (9) CT165 (Hypothetical protein) and (10) CT713 (PorB).

Preferably the immunogenic compositions of the present invention comprise one or more antigens selected from a “fourth antigen” group consisting of: (1) CT559 (YscJ); (2) CT600 (Pal); (3) CT541 (Mip); (4) CT623 (CHLPN 76 kDA homologue) (5) CT700 (Hypothetical protein). (6) CT266 (Hypothetical protein); (7) CT077 (Hypothetical protein); (8) CT456 (Hypothetical protein); (9) CT165 (Hypothetical protein) and (10) CT713 (PorB); or other combinations thereof in combination with an immunoregulatory agent which is selected from the group consisting of CFA, Alum, CpG, AlOH, Alum and CpG, AlOH and CpG LTK63 and LTK63 and CpG.

Still more preferably the immunogenic compositions of the present invention comprise one or more antigens selected from a “fourth antigen” group consisting of: (1) CT559 (YscJ); (2) CT600 (Pal); (3) CT541 (Mip); (4) CT623 (CHLPN 76 kDA homologue) (5) CT700 (Hypothetical protein). (6) CT266 (Hypothetical protein); (7) CT077 (Hypothetical protein); (8) CT456 (Hypothetical protein); (9) CT165 (Hypothetical protein) and (10) CT713 (PorB); or other combinations thereof in combination with Alum and CpG or AlOH and CpG.

The immunogenic compositions of the present invention may comprise one or more antigens selected from a “fifth antigen” group consisting of: (1) CT082 (hypothetical); (2) CT181 (Hypothetical); (3) CT050 (Hypothetical); (4) CT157 (Phospholipase D superfamily); and (5) CT128 (AdK adenylate cyclase).

Preferably the immunogenic compositions of the present invention comprise one or more antigens selected from a “fifth antigen” group consisting of: (1) CT082 (hypothetical); (2) CT181 (Hypothetical); (3) CT050 (Hypothetical); (4) CT157 (Phospholipase D superfamily); and (5) CT128 (AdK adenylate cyclase) or other combinations thereof in combination with an immunoregulatory agent which is selected from the group consisting of CFA, Alum, CpG, AlOH, Alum and CpG, AlOH and CpG, LTK63, LTK63 and CpG.

Still more preferably the immunogenic compositions of the present invention comprise one or more antigens selected from a “fifth antigen” group consisting of: (1) CT082 (hypothetical); (2) CT181 (Hypothetical); (3) CT050 (Hypothetical); (4) CT157 (Phospholipase D superfamily); and (5) CT128 (AdK adenylate cyclase); or other combinations thereof in combination with Alum and CpG or AlOH and CpG.

The immunogenic compositions of the present invention may comprise one or more antigens selected from a “sixth antigen” group consisting of: (1) CT153 (Hypothetical); (2) CT262 (Hypothetical); (3) CT276 (Hypothetical); (4) CT296 (Hypothetical); (5) CT372 (Hypothetical); (6) CT412 (PmpA); (7) CT480 (OligoPeptide Binding Protein); (8) CT548 (Hypothetical); (9) CT043 (Hypothetical); (10) CT635 (Hypothetical); (11) CT859 (Metalloprotease); (12) CT671 (Hypothetical); (13) CT016 (Hypothetical); (14) CT017 (Hypothetical); (15) CT043 (Hypothetical); (16) CT082 (Hypothetical); (17) CT548 (Hypothetical); (19) CT089 (Low Calcium Response Element); (20) CT812 (PmpD) and (21) CT869 (PmpE); or other combinations thereof.

Preferably the immunogenic compositions of the present invention comprise one or more antigens selected from a “sixth antigen” group consisting of: (1) CT153 (Hypothetical); (2) CT262 (Hypothetical); (3) CT276 (Hypothetical); (4) CT296 (Hypothetical); (5) CT372 (Hypothetical); (6) CT412 (PmpA); (7) CT480 (OligoPeptide Binding Protein); (8) CT548 (Hypothetical); (9) CT043 (Hypothetical); (10) CT635 (Hypothetical); (11) CT859 (Metalloprotease); (12) CT671 (Hypothetical); (13) CT016 (Hypothetical); (14) CT017 (Hypothetical); (15) CT043 (Hypothetical); (16) CT082 (Hypothetical); (17) CT548 (Hypothetical); (19) CT089 (Low Calcium Response Element); (20) CT812 (PmpD) and (21) CT869 (PmpE); or other combinations thereof in combination with an immunoregulatory agent which is selected from the group consisting of CFA, Alum, CpG, AlOH, Alum and CpG, AlOH and CpG, LTK63, LTK63 and CpG.

Still more preferably the immunogenic compositions of the present invention comprise one or more antigens selected from a “sixth antigen” group consisting of: (1) CT153 (Hypothetical); (2) CT262 (Hypothetical); (3) CT276 (Hypothetical); (4) CT296 (Hypothetical); (5) CT372 (Hypothetical); (6) CT412 (PmpA); (7) CT480 (OligoPeptide Binding Protein); (8) CT548 (Hypothetical); (9) CT043 (Hypothetical); (10) CT635 (Hypothetical); (11) CT859 (Metalloprotease); (12) CT671 (Hypothetical); (13) CT016 (Hypothetical); (14) CT017 (Hypothetical); (15) CT043 (Hypothetical); (16) CT082 (Hypothetical); (17) CT548 (Hypothetical); (19) CT089 (Low Calcium Response Element); (20) CT812 (PmpD) and (21) CT869 (PmpE); or other combinations thereof in combination with Alum and CpG or AlOH and CpG.

FACS analyses, Western Blot analyses and In-vitro neutralisation analyses—carried out as described in the Examples and in WO 03/049762—demonstrate that proteins in the first, second, third, fourth, fifth and antigen groups are surface-exposed and immunoaccessible proteins and are useful immunogens. These properties are not evident from the sequence alone. In addition, proteins described in the fourth, fifth and sixth antigen groups (as well as the first, second, third and fourth antigen groups) which are described as “hypothetical” typically have no known cellular location or biological function and generally, do not have any bacterial homologue, such as a Chlamydia pneumoniae homologues.

The immunogenic compositions of the present invention may comprise one or more antigens selected from a “third antigen” group consisting of: (1) Pgp3; (2) CT412 (PmpA); (3) CT413 (PmpB); (4) CT414 (PmpC); (5) CT812 (PmpD); (6) CT869 (PmpE); (7) CT870 (PmpF); (8) CT871 (PmpG); (9) CT872 (PmpH); (10) PmpI; (11) CT681 (MOMP); (12) CT529 (Cap1); (13) Hsp-60; and (14) CT443 (OmcB).

Preferably the immunogenic compositions of the present invention comprise one or more antigens selected from a “third antigen” group consisting of: (1) Pgp3; (2) CT412 (PmpA); (3) CT413 (PmpB); (4) CT414 (PmpC); (5) CT812 (PmpD); (6) CT869 (PmpE); (7) CT870 (PmpF); (8) CT871 (PmpG); (9) CT872 (PmpH); (10) PmpI; (11) CT681 (MOMP); (12) CT529 (Cap1); (13) Hsp-60; and (14) CT443 (OmcB); in combination with an immunoregulatory agent which is selected from the group consisting of CFA, Alum, CpG, AlOH, Alum and CpG, AlOH and CpG, LTK63 and LTK63 and CpG.

Still more preferably the immunogenic compositions of the present invention comprise one or more antigens selected from a “third antigen” group consisting of: (1) Pgp3; (2) CT412 (PmpA); (3) CT413 (PmpB); (4) CT414 (PmpC); (5) CT812 (PmpD); (6) CT869 (PmpE); (7) CT870 (PmpF); (8) CT871 (PmpG); (9) CT872 (PmpH); (10) PmpI; (11) CT681 (MOMP); (12) CT529 (Cap1); (13) Hsp-60; (14) CT443 (OmcB); in combination with Alum and CpG or AlOH and CpG.

The immunogenic compositions of the present invention may comprise the Pmp antigens: (2) CT412 (PmpA); (3) CT413 (PmpB); (4) CT414 (PmpC); (5) CT812 (PmpD); (6) CT869 (PmpE); (7) CT870 (PmpF); (8) CT871 (PmpG); (9) CT872 (PmpH); and (10) PmpI

Preferably the immunogenic compositions of the present invention comprise the PmP antigens (2) CT412 (PmpA); (3) CT413 (PmpB); (4) CT414 (PmpC); (5) CT812 (PmpD); (6) CT869 (PmpE); (7) CT870 (PmpF); (8) CT871 (PmpG); (9) CT872 (PmpH); and (10) PmpI in combination with an immunoregulatory agent which is selected from the group consisting of CFA, Alum, CpG, AlOH, Alum and CpG, AlOH and CpG, LTK63 and LTK63 and CpG.

Still more preferably the immunogenic compositions of the present invention comprise the PmP antigens (2) CT412 (PmpA); (3) CT413 (PmpB); (4) CT414 (PmpC); (5) CT812 (PmpD); (6) CT869 (PmpE); (7) CT870 (PmpF); (8) CT871 (PmpG); (9) CT872 (PmpH); and (10) PmpI; in combination with Alum and CpG or AlOH and CpG.

The immunogenic compositions of the present invention may comprise one or more antigens selected from a “first or second antigen” group consisting of: (1) 045 (PepA); (2) CT089 (LcrE); (3) CT396 (DnaK); (4) CT398 (Hypothetical); (5) CT381 (ArtJ); (6) CT242 (OmpH-like); (7) CT316 (L7/L12); (8) CT444 (OmcA); (9) CT467 (AtoS); (10) CT547 (Hypothetical); (11) CT587 (Enolase); (12) CT823 (HtrA); (13) CT761 (MurG).

Preferably the immunogenic compositions of the present invention comprise one or more antigens selected from a “first or second antigen” group consisting of: (1) 045 (PepA); (2) CT089 (LcrE); (3) CT396 (DnaK); (4) CT398 (Hypothetical); (5) CT381 (ArtJ); (6) CT242 (OmpH-like); (7) CT316 (L7/L12); (8) CT444 (OmcA); (9) CT467 (AtoS); (10) CT547 (Hypothetical); (11) CT587 (Enolase); (12) CT823 (HtrA); (13) CT761 (MurG); in combination with an immunoregulatory agent which is selected from the group consisting of CFA, Alum, CpG, AlOH, Alum and CpG, AlOH and CpG, LTK63 and LTK63 and CpG.

Still more preferably the immunogenic compositions of the present invention comprise one or more antigens selected from a “first or second antigen” group consisting of: (1) 045 (PepA); (2) CT089 (LcrE); (3) CT396 (DnaK); (4) CT398 (Hypothetical); (5) CT381 (ArtJ); (6) CT242 (OmpH-like); (7) CT316 (L7/L12); (8) CT444 (OmcA); (9) CT467 (AtoS); (10) CT547 (Hypothetical); (11) CT587 (Enolase); (12) CT823 (HtrA); (13) CT761 (MurG in combination with Alum and CpG or AlOH and CpG.

Preferably the immunogenic composition comprises: CT089 and CT381 and CT396 and CT548.

Preferably the immunogenic composition comprises: CT089 and CT381 and CT396 and CT548 in combination with an immunoregulatory agent which is selected from the group consisting of CFA, Alum, CpG, AlOH, Alum and CpG, AlOH and CpG, LTK63 and LTK63 and CpG.

Preferably the immunogenic composition comprises: CT089 and CT381 and CT396 and CT548 in combination with Alum and CpG or AlOH and CpG

Preferably the immunogenic compositions of the present invention comprises: CT045 in combination with Alum and CpG or AlOH and CpG.

Preferably the immunogenic compositions of the present invention comprises: CT089 in combination with Alum and CpG or AlOH and CpG.

Preferably the immunogenic compositions of the present invention comprises: CT396 combination with Alum and CpG or AlOH and CpG.

Preferably the immunogenic compositions of the present invention comprises: CT398 in combination with Alum and CpG or AlOH and CpG.

Preferably the immunogenic compositions of the present invention comprises: CT381 in combination with Alum and CpG or AlOH and CpG.

Preferably the immunogenic compositions of the present invention comprises: CT242 in combination with Alum and CpG or AlOH and CpG.

Preferably the immunogenic compositions of the present invention comprises: CT316 in combination with Alum and CpG or AlOH and CpG.

Preferably the immunogenic compositions of the present invention comprises: CT444 in combination with Alum and CpG or AlOH and CpG.

Preferably the immunogenic compositions of the present invention comprises: CT467 in combination with Alum and CpG or AlOH and CpG.

Preferably the immunogenic compositions of the present invention comprises: CT587 in combination with Alum and CpG or AlOH and CpG.

Preferably the immunogenic compositions of the present invention comprises: CT823 in combination with Alum and CpG or AlOH and CpG.

Preferably the immunogenic compositions of the present invention comprises: CT761 in combination with Alum and CpG or AlOH and CpG.

### Fusion Proteins

The Chlamydia trachomatis antigens used in the invention may be present in the composition as individual separate polypeptides. Generally, the recombinant fusion proteins of the present invention are prepared as a GST-fusion protein and/or a His-tagged fusion protein.

However, preferably, at least two (i.e. 2, 3, 4, 5, 6, 7, 8, 9, 10, 11, 12, 13, 14, 15, 16, 17, 18, 19 or 20) of the antigens are expressed as a single polypeptide chain (a ‘hybrid’ polypeptide). Hybrid polypeptides offer two principal advantages: first, a polypeptide that may be unstable or poorly expressed on its own can be assisted by adding a suitable hybrid partner that overcomes the problem; second, commercial manufacture is simplified as only one expression and purification need be employed in order to produce two polypeptides which are both antigenically useful.

The hybrid polypeptide may comprise two or more polypeptide sequences from the first antigen group. Accordingly, the invention includes a composition comprising a first amino acid sequence and a second amino acid sequence, wherein said first and second amino acid sequences are selected from a Chlamydia trachomatis antigen or a fragment thereof of the first antigen group. Preferably, the first and second amino acid sequences in the hybrid polypeptide comprise different epitopes.

The hybrid polypeptide may comprise two or more polypeptide sequences from the second antigen group. Accordingly, the invention includes a composition comprising a first amino acid sequence and a second amino acid sequence, wherein said first and second amino acid sequences are selected from a Chlamydia trachomatis antigen or a fragment thereof of the second antigen group. Preferably, the first and second amino acid sequences in the hybrid polypeptide comprise difference epitopes.

The hybrid polypeptide may comprise one or more polypeptide sequences from the first antigen group and one or more polypeptide sequences from the second antigen group. Accordingly, the invention includes a composition comprising a first amino acid sequence and a second amino acid sequence, said first amino acid sequence selected from a Chlamydia trachomatis antigen or a fragment thereof from the first antigen group and said second amino acid sequence selected from a Chlamydia trachomatis antigen or a fragment thereof from the second antigen group. Preferably, the first and second amino acid sequences in the hybrid polypeptide comprise difference epitopes.

The hybrid polypeptide may comprise one or more polypeptide sequences from the first antigen group and one or more polypeptide sequences from the third antigen group. Accordingly, the invention includes a composition comprising a first amino acid sequence and a second amino acid sequence, said first amino acid sequence selected from a Chlamydia trachomatis antigen or a fragment thereof from the first antigen group and said second amino acid sequence selected from a Chlamydia trachomatis antigen or a fragment thereof from the third antigen group. Preferably, the first and second amino acid sequences in the hybrid polypeptide comprise difference epitopes.

The hybrid polypeptide may comprise one or more polypeptide sequences from the second antigen group and one or more polypeptide sequences from the third antigen group. Accordingly, the invention includes a composition comprising a first amino acid sequence and a second amino acid sequence, said first amino acid sequence selected from a Chlamydia trachomatis antigen or a fragment thereof from the second antigen group and said second amino acid sequence selected from a Chlamydia trachomatis antigen or a fragment thereof from the third antigen group. Preferably, the first and second amino acid sequences in the hybrid polypeptide comprise difference epitopes.

Hybrids consisting of amino acid sequences from two, three, four, five, six, seven, eight, nine, or ten Chlamydia trachomatis antigens are preferred. In particular, hybrids consisting of amino acid sequences from two, three, four, or five Chlamydia trachomatis antigens are preferred. Different hybrid polypeptides may be mixed together in a single formulation. Within such combinations, a Chlamydia trachomatis antigen may be present in more than one hybrid polypeptide and/or as a non-hybrid polypeptide. It is preferred, however, that an antigen is present either as a hybrid or as a non-hybrid, but not as both.

Two-antigen hybrids for use in the invention may comprise: (1) PepA & LcrE; (2) PepA & OmpH-like; (3) PepA & L7/L12; (4) PepA & ArtJ; (5) PepA & DnaK; (6) PepA & CT398; (7) PepA & OmcA; (8) PepA & AtoS; (9) PepA & CT547; (10) PepA & Eno; (11) PepA & HrtA; (12) PepA & MurG; (13) LcrE & OmpH-like; (14) LcrE & L7/L12; (15) LcrE & ArtJ; (16) LcrE & DnaK; (17) LcrE & CT398; (18) LcrE & OmcA; (19) LcrE & AtoS; (20) LcrE & CT547; (21) LcrE & Eno; (22) LcrE & HrtA; (23) LcrE & MurG; (24) OmpH-like & L7/L12; (25) OmpH-like & ArtJ; (26) OmpH-like & DnaK; (27) OmpH-like & CT398; (28) OmpH-like & OmcA; (29) OmpH-like & AtoS; (30) OmpH-like & CT547; (31) OmpH-like & Eno; (32) OmpH-like & HrtA; (33) OmpH-like & MurG; (34) L7/L12 & ArtJ; (35) L7/L12 & DnaK; (36) L7/L12 & CT398; (37) L7/L12 & OmcA; (38) L7/L12 & AtoS; (39) L7/L12 & CT547; (40) L7/L12 & Eno; (41) L7/L12 & HrtA; (42) L7/L12 & MurG; (43) ArtJ & DnaK; (44) ArtJ & CT398; (45) ArtJ & OmcA; (46) ArtJ & AtoS; (47) ArtJ & CT547; (48) ArtJ & Eno; (49) ArtJ & HrtA; (50) ArtJ & MurG; (51) DnaK & CT398; (52) DnaK & OmcA; (53) DnaK & AtoS; (54) DnaK & CT547; (55) DnaK & Eno; (56) DnaK & HrtA; (57) DnaK & MurG; (58) CT398 & OmcA; (59) CT398 & AtoS; (60) CT398 & CT547; (61) CT398 & Eno; (62) CT398 & HrtA; (63) CT398 & MurG; (64) OmcA & AtoS; (65) OmcA & CT547; (66) OmcA & Eno; (67) OmcA & HrtA; (68) OmcA & MurG; (69) AtoS & CT547; (70) AtoS & Eno; (71) AtoS & HrtA; (72) AtoS & MurG; (73) CT547 & Eno; (74) CT547 & HrtA; (75) CT547 & MurG; (76) Eno & HrtA; (77) Eno & MurG; (78) HrtA & MurG or (79) PmpD (CT812) and Hypothetical (CT082).

Two antigen hybrids for use in the present invention may also comprise combinations of antigens selected from the third, fourth, fifth and sixth antigen groups.

Hybrid polypeptides can be represented by the formula NH₂-A-{-X-L-}ₙ-B—COOH, wherein: X is an amino acid sequence of a Chlamydia trachomatis antigen or a fragment thereof from the first antigen group, the second antigen group or the third antigen group; L is an optional linker amino acid sequence; A is an optional N-terminal amino acid sequence; B is an optional C-terminal amino acid sequence; and n is 2, 3, 4, 5, 6, 7, 8, 9, 10, 11, 12, 13, 14 or 15.

If a —X— moiety has a leader peptide sequence in its wild-type form, this may be included or omitted in the hybrid protein. In some embodiments, the leader peptides will be deleted except for that of the —X— moiety located at the N-terminus of the hybrid protein i.e. the leader peptide of X₁ will be retained, but the leader peptides of X₂ . . . Xₙ will be omitted. This is equivalent to deleting all leader peptides and using the leader peptide of X₁ as moiety -A-.

For each n instances of {-X-L-}, linker amino acid sequence -L- may be present or absent. For instance, when n=2 the hybrid may be NH₂—X₁-L₁-X₂-L₂-COOH, NH₂—X₁—X₂—COOH, NH₂—X₁-L₁-X₂—COOH, NH₂—X₁—X₂-L₂-COOH, etc. Linker amino acid sequence(s) -L- will typically be short (e.g. 20 or fewer amino acids i.e. 19, 18, 17, 16, 15, 14, 13, 12, 11, 10, 9, 8, 7, 6, 5, 4, 3, 2, 1). Examples comprise short peptide sequences which facilitate cloning, poly-glycine linkers (i.e. comprising Glyₙ where n=2, 3, 4, 5, 6, 7, 8, 9, 10 or more), and histidine tags (i.e. Hisₙ where n=3, 4, 5, 6, 7, 8, 9, 10 or more). Other suitable linker amino acid sequences will be apparent to those skilled in the art. A useful linker is GSGGGG (SEQ ID 1), with the Gly-Ser dipeptide being formed from a BamHI restriction site, thus aiding cloning and manipulation, and the (Gly)₄ tetrapeptide being a typical poly-glycine linker.

-A- is an optional N-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct protein trafficking, or short peptide sequences which facilitate cloning or purification (e.g. histidine tags i.e. Hisₙ where n=3, 4, 5, 6, 7, 8, 9, 10 or more). Other suitable N-terminal amino acid sequences will be apparent to those skilled in the art. If X₁ lacks its own N-terminus methionine, -A- is preferably an oligopeptide (e.g. with 1, 2, 3, 4, 5, 6, 7 or 8 amino acids) which provides a N-terminus methionine.

—B— is an optional C-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include sequences to direct protein trafficking, short peptide sequences which facilitate cloning or purification (e.g. comprising histidine tags i.e. Hisₙ where n=3, 4, 5, 6, 7, 8, 9, 10 or more), or sequences which enhance protein stability. Other suitable C-terminal amino acid sequences will be apparent to those skilled in the art. Most preferably, n is 2 or 3.

The invention also provides nucleic acid encoding hybrid polypeptides of the invention. Furthermore, the invention provides nucleic acid which can hybridise to this nucleic acid, preferably under “high stringency” conditions (e.g. 65° C. in a 0.1×SSC, 0.5% SDS solution). Polypeptides of the invention can be prepared by various means (e.g. recombinant expression, purification from cell culture, chemical synthesis, etc.) and in various forms (e.g. native, fusions, non-glycosylated, lipidated, etc.). They are preferably prepared in substantially pure form (i.e. substantially free from other chlamydial or host cell proteins).

Nucleic acid according to the invention can be prepared in many ways (e.g. by chemical synthesis, from genomic or cDNA libraries, from the organism itself, etc.) and can take various forms (e.g. single stranded, double stranded, vectors, probes, etc.). They are preferably prepared in substantially pure form (i.e. substantially free from other chlamydial or host cell nucleic acids).

The term “nucleic acid” includes DNA and RNA, and also their analogues, such as those containing modified backbones (e.g. phosphorothioates, etc.), and also peptide nucleic acids (PNA), etc. The invention includes nucleic acid comprising sequences complementary to those described above (e.g. for antisense or probing purposes).

The invention also provides a process for producing a polypeptide of the invention, comprising the step of culturing a host cell transformed with nucleic acid of the invention under conditions which induce polypeptide expression.

The invention provides a process for producing a polypeptide of the invention, comprising the step of synthesizing at least part of the polypeptide by chemical means.

The invention provides a process for producing nucleic acid of the invention, comprising the step of amplifying nucleic acid using a primer-based amplification method (e.g. PCR).

The invention provides a process for producing nucleic acid of the invention, comprising the step of synthesizing at least part of the nucleic acid by chemical means.

### Strains

Preferred polypeptides of the invention comprise an amino acid sequence found in C. trachomatis serovar D, or in one or more of an epidemiologically prevalent serotype.

Where hybrid polypeptides are used, the individual antigens within the hybrid (i.e. individual —X— moieties) may be from one or more strains. Where n=2, for instance, X₂ may be from the same strain as X₁ or from a different strain. Where n=3, the strains might be (i) X₁═X₂═X₃ (ii) X₁═X₂≠X₃ (iii) X₁≠X₂═X₃ (iv) X₁≠X₂≠X₃ or (v) X₁═X₃≠X₂, etc.

### Heterologous Host

Whilst expression of the polypeptides of the invention may take place in Chlamydia, the invention preferably utilises a heterologous host. The heterologous host may be prokaryotic (e.g. a bacterium) or eukaryotic. It is preferably E. coli, but other suitable hosts include Bacillus subtilis, Vibrio cholerae, Salmonella typhi, Salmonella typhimurium, Neisseria lactamica, Neisseria cinerea, Mycobacteria (e.g. M. tuberculosis), yeasts, etc.

### Immunogenic Compositions and Medicaments

Compositions of the invention are preferably immunogenic compositions, and are more preferably vaccine compositions. The pH of the composition is preferably between 6 and 8, preferably about 7. The pH may be maintained by the use of a buffer. The composition may be sterile and/or pyrogen-free. The composition may be isotonic with respect to humans.

Vaccines according to the invention may either be prophylactic (i.e. to prevent infection) or therapeutic (i.e. to treat infection), but will typically be prophylactic. Accordingly, the invention includes a method for the therapeutic or prophylactic treatment of Chlamydia trachomatis infection in an animal susceptible to chlamydial infection comprising administering to said animal a therapeutic or prophylactic amount of the immunogenic compositions of the invention. Preferably, the immunogenic composition comprises a combination of Chlamydia trachomatis antigens, said combination selected from the group consisting of two, three, four, or all five Chlamydia trachomatis antigens of the first antigen group. Still more preferably, the combination consists of all five Chlamydia trachomatis antigens of the first antigen group.

Alternatively, the immunogenic composition comprises a combination of Chlamydia trachomatis antigens, said combination selected from the group consisting of two, three, four, five, six, seven, eight, nine, ten, eleven, twelve, or thirteen Chlamydia trachomatis antigens selected from the second antigen group. Preferably, the combination is selected from the group consisting of three, four, or five Chlamydia trachomatis antigens selected from the second antigen group. Still more preferably, the combination consists of five Chlamydia trachomatis antigens selected from the second antigen group.

Alternatively, the immunogenic composition comprises a combination of Chlamydia trachomatis antigens, said combination consisting of two, three, four, or five Chlamydia trachomatis antigens of the first antigen group and one, two, three, four, five or six Chlamydia trachomatis antigens of the third antigen group. Preferably, the combination consists of three, four or five Chlamydia trachomatis antigens of the first antigen group and one, two, three, four, five or six Chlamydia trachomatis antigens of the third antigen group.

Alternatively, the immunigenic composition comprises a combination of Chlamydia trachomatis antigens, said combination consisting of two, three, four, five, six, seven, eight, nine, ten, eleven, twelve or thirteen Chlamydia trachomatis antigens of the second antigen group and one, two, three, four, five or six Chlamydia trachomatis antigens of the third antigen group. Preferably, the combination is selected from the group consisting of three, four, or five Chlamydia trachomatis antigens from the second antigen group and three, four or five Chlamydia trachomatis from the third antigen group. Still more preferably, the combination consists of five Chlamydia trachomatis antigens from the second antigen group and three, four or five Chlamydia trachomatis antigens of the third antigen group.

Alternatively, the immunigenic composition comprises a combination of Chlamydia trachomatis antigens, said combination consisting of two, three, four; five, six, seven, eight, nine or ten Chlamydia trachomatis antigens of the fourth antigen group and one, two, three, four or five Chlamydia trachomatis antigens of the fifth antigen group and one, two, three, four, five, six, seven, eight, nine, ten, eleven, twelve, thirteen, fourteen, fifteen, sixteen, seventeen, eighteen, nineteen, twenty or twentyone antigens of the sixth antigen group. Preferably, the combination is selected from the group consisting of three, four, or five Chlamydia trachomatis antigens from the fourth antigen group and three, four or five Chlamydia trachomatis from the fifth antigen group. Still more preferably, the combination consists of five Chlamydia trachomatis antigens from the fourth antigen group and three, four or five Chlamydia trachomatis antigens of the fifth antigen group.

The invention also comprises an immunogenic composition comprising one or more immunoregulatory agents. Preferably, one or more of the immunoregulatory agents include an adjuvant. The adjuvant may be selected from one or more of the group consisting of a TH1 adjuvant and TH2 adjuvant, further discussed below. The adjuvant may be selected from the group consisting of a mineral salt, such as an aluminium salt and an oligonucleotide containing a CpG motif. Most preferably, the immunogenic composition includes both an aluminium salt and an oligonucleotide containing a CpG motif. Alternatively, the immunogenic composition includes an ADP ribosylating toxin, such as a detoxified ADP ribosylating toxin and an oligonucleotide containing a CpG motif.

The compositions of the invention will preferably elicit both a cell mediated immune response as well as a humoral immune response in order to effectively address a Chlamydia intracellular infection. This immune response will preferably induce long lasting (eg neutralising) antibodies and a cell mediated immunity that can quickly respond upon exposure to Chlamydia.

Two types of T cells, CD4 and CD8 cells, are generally thought necessary to initiate and/or enhance cell mediated immunity and humoral immunity. CD8 T cells can express a CD8 co-receptor and are commonly referred to as Cytotoxic T lymphocytes (CTLs). CD8 T cells are able to recognized or interact with antigens displayed on MHC Class I molecules.

CD4 T cells can express a CD4 co-receptor and are commonly referred to as T helper cells. CD4 T cells are able to recognize antigenic peptides bound to MHC class II molecules. Upon interaction with a MHC class II molecule, the CD4 cells can secrete factors such as cytokines. These secreted cytokines can activate B cells, cytotoxic T cells, macrophages, and other cells that participate in an immune response. Helper T cells or CD4+ cells can be further divided into two functionally distinct subsets: TH1 phenotype and TH2 phenotypes which differ in their cytokine and effector function.

Activated TH1 cells enhance cellular immunity (including an increase in antigen-specific CTL production) and are therefore of particular value in responding to intracellular infections. Activated TH1 cells may secrete one or more of IL-2, IFN-gamma, and TNF-beta. A TH1 immune response may result in local inflammatory reactions by activating macrophages, NK (natural killer) cells, and CD8 cytotoxic T cells (CTLs). A TH1 immune response may also act to expand the immune response by stimulating growth of B and T cells with IL-12. TH1 stimulated B cells may secrete IgG2a.

Activated TH2 cells enhance antibody production and are therefore of value in responding to extracellular infections. Activated TH2 cells may secrete one or more of IL-4, IL-5, IL-6, and IL-10. A TH2 immune response may result in the production of IgG1, IgE, IgA and memory B cells for future protection.

An enhanced immune response may include one or more of an enhanced TH1 immune response and a TH2 immune response.

An enhanced TH1 immune response may include one or more of an increase in CTLs, an increase in one or more of the cytokines associated with a TH1 immune response (such as IL-2, IFN-gamma, and TNF-beta), an increase in activated macrophages, an increase in NK activity, or an increase in the production of IgG2a. Preferably, the enhanced TH1 immune response will include an increase in IgG2a production.

A TH1 immune response may be elicited using a TH1 adjuvant. A TH1 adjuvant will generally elicit increased levels of IgG2a production relative to immunization of the antigen without adjuvant. TH1 adjuvants suitable for use in the invention may include for example saponin formulations, virosomes and virus like particles, non-toxic derivatives of enterobacterial lipopolysaccharide (LPS), immunostimulatory oligonucleotides. Immunostimulatory oligonucleotides, such as oligonucleotides containing a CpG motif, are preferred TH1 adjuvants for use in the invention.

An enhanced TH2 immune response may include one or more of an increase in one or more of the cytokines associated with a TH2 immune response (such as IL-4, IL-5, IL-6 and IL-10), or an increase in the production of IgG1, IgE, IgA and memory B cells. Preferably, the enhanced TH2 immune response will include an increase in IgG1 production.

A TH2 immune response may be elicited using a TH2 adjuvant. A TH2 adjuvant will generally elicit increased levels of IgG1 production relative to immunization of the antigen without adjuvant. TH2 adjuvants suitable for use in the invention include, for example, mineral containing compositions, oil-emulsions, and ADP-ribosylating toxins and detoxified derivatives thereof. Mineral containing compositions, such as aluminium salts are preferred TH2 adjuvants for use in the invention.

Preferably, the invention includes a composition comprising a combination of a TH1 adjuvant and a TH2 adjuvant. Preferably, such a composition elicits an enhanced TH1 and an enhanced TH2 response, i.e., an increase in the production of both IgG1 and IgG2a production relative to immunization without an adjuvant. Still more preferably, the composition comprising a combination of a TH1 and a TH2 adjuvant elicits an increased TH1 and/or an increased TH2 immune response relative to immunization with a single adjuvant (i.e., relative to immunization with a TH1 adjuvant alone or immunization with a TH2 adjuvant alone).

As discussed further in the Examples, use of the combination of a mineral salt, such as an aluminium salt, and an oligonucleotide containing a CpG motif provide for an enhanced immune response. This improved immune response is wholly unexpected and could not be predicted from the use of either agent alone. The invention therefore includes an oligonucleotide containing a CpG motif, a mineral salt such as an aluminium salt, and an antigen associated with a sexually transmissible disease, such as a Chlamydia trachomatis antigen. Further examples of antigens associated with a sexually transmissible disease are discussed further below.

The invention also provides a composition of the invention for use as a medicament. The medicament is preferably able to raise an immune response in a mammal (i.e. it is an immunogenic composition) and is more preferably a vaccine. The invention also provides the use of the compositions of the invention in the manufacture of a medicament for raising an immune response in a mammal. The medicament is preferably a vaccine.

The immune response may be one or both of a TH1 immune response and a TH2 response. Preferably, immune response provides for one or both of an enhanced TH1 response and an enhanced TH2 response.

The enhanced immune response may be one or both of a systemic and a mucosal immune response. Preferably, the immune response provides for one or both of an enhanced systemic and an enhanced mucosal immune response. Preferably the mucosal immune response is a TH2 immune response. Preferably, the mucosal immune response includes an increase in the production of IgA.

The invention also provides for a kit comprising a first component comprising a combination of Chlamydia trachomatis antigens. The combination of Chlamydia trachomatis antigens may be one or more of the immunogenic compositions of the invention. The kit may further include a second component comprising one or more of the following: instructions, syringe or other delivery device, adjuvant, or pharmaceutically acceptable formulating solution.

The invention also provides a delivery device pre-filled with the immunogenic compositions of the invention.

The invention also provides a method for raising an immune response in a mammal comprising the step of administering an effective amount of a composition of the invention. The immune response is preferably protective and preferably involves antibodies and/or cell-mediated immunity. Preferably, the immune response includes one or both of a TH1 immune response and a TH2 immune response. The method may raise a booster response.

The mammal is preferably a human. Where the vaccine is for prophylactic use, the human is preferably a child (e.g. a toddler or infant) or a teenager; where the vaccine is for therapeutic use, the human is preferably a teenager or an adult. A vaccine intended for children may also be administered to adults e.g. to assess safety, dosage, immunogenicity, etc.

One way of checking efficacy of therapeutic treatment involves monitoring C. trachomatis infection after administration of the compositions of the invention. One way of checking efficacy of prophylactic treatment involves monitoring immune responses both systemically (such as monitoring the level of IgG1 and IgG2a production) and mucosally (such as monitoring the level of IgA production) against the Chlamydia trachomatis antigens in the compositions of the invention after administration of the composition. Typically, serum Chlamydia specific antibody responses are determined post-immunisation but pre-challenge whereas mucosal Chlamydia specific antibody body responses are determined post-immunisation and post-challenge.

These uses and methods are preferably for the prevention and/or treatment of a disease caused by a Chlamydia (e.g. trachoma, pelvic inflammatory disease, epididymitis, infant pneumonia, etc.). The compositions may also be effective against C. pneumoniae.

The vaccine compositions of the present invention can be evaluated in in vitro and in vivo animal models prior to host, e.g., human, administration. For example, in vitro neutralization by Peterson et al (1988) is suitable for testing vaccine compositions directed toward Chlamydia trachomatis.

One example of such an in vitro test is described as follows. Hyper-immune antisera is diluted in PBS containing 5% guinea pig serum, as a complement source. Chlamydia trachomatis (10⁴ IFU; inclusion forming units) are added to the antisera dilutions. The antigen-antibody mixtures are incubated at 37° C. for 45 minutes and inoculated into duplicate confluent Hep-2 or HeLa cell monolayers contained in glass vials (e.g., 15 by 45 mm), which have been washed twice with PBS prior to inoculation. The monolayer cells are infected by centrifugation at 1000×g for 1 hour followed by stationary incubation at 37° C. for 1 hour. Infected monolayers are incubated for 48 or 72 hours, fixed and stained with Chlamydia specific antibody, such as anti-MOMP. Inclusion-bearing cells are counted in ten fields at a magnification of 200×. Neutralization titer is assigned on the dilution that gives 50% inhibition as compared to control monolayers/IFU.

The efficacy of vaccine compositions can also be determined in vivo by challenging animal models of Chlamydia trachomatis infection, e.g., guinea pigs or mice, with the vaccine compositions. For example, in vivo vaccine composition challenge studies in the guinea pig model of Chlamydia trachomatis infection can be performed. A description of one example of this type of approach follows. Female guinea pigs weighing 450-500 g are housed in an environmentally controlled room with a 12 hour light-dark cycle and immunized with vaccine compositions via a variety of immunization routes. Post-vaccination, guinea pigs are infected in the genital tract with the agent of guinea pig inclusion conjunctivitis (GPIC), which has been grown in HeLa or McCoy cells (Rank et al. (1988)). Each animal receives approximately 1.4×10⁷ inclusion forming units (IFU) contained in 0.05 ml of sucrose-phosphate-glutamate buffer, pH 7.4 (Schacter, 1980). The course of infection monitored by determining the percentage of inclusion-bearing cells by indirect immunofluorescence with GPIC specific antisera, or by Giemsa-stained smear from a scraping from the genital tract (Rank et al 1988). Antibody titers in the serum is determined by an enzyme-linked immunosorbent assay.

Alternatively, in vivo vaccine compositions challenge studies can be performed in the murine model of Chlamydia trachomatis (Morrison et al 1995). A description of one example of this type of approach is as follows. Female mice 7 to 12 weeks of age receive 2.5 mg of depoprovera subcutaneously at 10 and 3 days before vaginal infection. Post-vaccination, mice are infected in the genital tract with 1,500 inclusion-forming units of Chlamydia trachomatis contained in 5 ml of sucrose-phosphate-glutamate buffer, pH 7.4. The course of infection is monitored by determining the percentage of inclusion-bearing cells by indirect immunofluorescence with Chlamydia trachomatis specific antisera, or by a Giemsa-stained smear from a scraping from the genital tract of an infected mouse. The presence of antibody titers in the serum of a mouse is determined by an enzyme-linked immunosorbent assay.

Compositions of the invention will generally be administered directly to a patient. Direct delivery may be accomplished by parenteral injection (e.g. subcutaneously, intraperitoneally, intravenously, intramuscularly, or to the interstitial space of a tissue), or mucosally, such as by rectal, oral (e.g. tablet, spray), vaginal, topical, transdermal (See e.g. WO99/27961) or transcutaneous (See e.g. WO02/074244 and WO02/064162), intranasal (See e.g. WO03/028760), ocular, aural, pulmonary or other mucosal administration.

The invention may be used to elicit systemic and/or mucosal immunity, preferably to elicit an enhanced systemic and/or mucosal immunity.

Preferably the enhanced systemic and/or mucosal immunity is reflected in an enhanced TH1 and/or TH2 immune response. Preferably, the enhanced immune response includes an increase in the production of IgG1 and/or IgG2a and/or IgA.

Dosage treatment can be a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc.

Chlamydial infections affect various areas of the body and so the compositions of the invention may be prepared in various forms. For example, the compositions may be prepared as injectables, either as liquid solutions or suspensions. Solid forms suitable for solution in, or suspension in, liquid vehicles prior to injection can also be prepared (e.g. a lyophilised composition or a spray-freeze dried composition). The composition may be prepared for topical administration e.g. as an ointment, cream or powder. The composition may be prepared for oral administration e.g. as a tablet or capsule, as a spray, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as drops. The composition may be in kit form, designed such that a combined composition is reconstituted just prior to administration to a patient. Such kits may comprise one or more antigens in liquid form and one or more lyophilised antigens.

Immunogenic compositions used as vaccines comprise an immunologically effective amount of antigen(s), as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesize antibodies, the degree of protection desired, the formulation of the vaccine, the treating doctor's assessment of the medical situation, and other relevant factors. It is expected that the amount will fall in a relatively broad range that can be determined through routine trials.

### Further Components of the Composition

The composition of the invention will typically, in addition to the components mentioned above, comprise one or more ‘pharmaceutically acceptable carriers’, which include any carrier that does not itself induce the production of antibodies harmful to the individual receiving the composition. Suitable carriers are typically large, slowly metabolised macromolecules such as proteins, polysaccharides, polylactic acids, polyglycolic acids, polymeric amino acids, amino acid copolymers, and lipid aggregates (such as oil droplets or liposomes). Such carriers are well known to those of ordinary skill in the art. The vaccines may also contain diluents, such as water, saline, glycerol, etc. Additionally, auxiliary substances, such as wetting or emulsifying agents, pH buffering substances, and the like, may be present. A thorough discussion of pharmaceutically acceptable excipients is available in Gennaro (2000) Remington: The Science and Practice of Pharmacy. 20th ed., ISBN: 0683306472.

### ImmunoRegulatory Agents

Vaccines of the present invention may be administered in conjunction with other immunoregulatory agents. In particular, compositions will usually include an adjuvant. Adjuvants for use with the invention include, but are not limited to, one or more of the following set forth below:

### A. Mineral Containing Compositions

Mineral containing compositions suitable for use as adjuvants in the invention include mineral salts, such as aluminum salts and calcium salts. The invention includes mineral salts such as hydroxides (e.g. oxyhydroxides), phosphates (e.g. hydroxyphosphates, orthophosphates), sulfates, etc. (e.g. see chapters 8 & 9 of Vaccine Design . . . (1995) eds. Powell & Newman. ISBN: 030644867X. Plenum.), or mixtures of different mineral compounds (e.g. a mixture of a phosphate and a hydroxide adjuvant, optionally with an excess of the phosphate), with the compounds taking any suitable form (e.g. gel, crystalline, amorphous, etc.), and with adsorption to the salt(s) being preferred. The mineral containing compositions may also be formulated as a particle of metal salt (WO00/23105).

Aluminum salts may be included in immunogenic compositions and/or vaccines of the invention such that the dose of Al³⁺ is between 0.2 and 1.0 mg per dose.

Preferably the adjuvant is alum, preferably an aluminium salt such as aluminium hydroxide (AlOH) or aluminium phosphate or aluminium sulfate. Still more preferably the adjuvant is aluminium hydroxide (AlOH).

Preferably a mineral salt, such as an aluminium salt, is combined with and another adjuvant, such as an oligonucleotide containing a CpG motif or an ADP ribosylating toxin. Still more preferably, the mineral salt is combined with an oligonucleotide containing a CpG motif.

### B. Oil-Emulsions

Oil-emulsion compositions suitable for use as adjuvants in the invention include squalene-water emulsions, such as MF59 (5% Squalene, 0.5% Tween 80, and 0.5% Span 85, formulated into submicron particles using a microfluidizer). See WO90/14837. See also, Frey et al., “Comparison of the safety, tolerability, and immunogenicity of a MF59-adjuvanted influenza vaccine and a non-adjuvanted influenza vaccine in non-elderly adults”, Vaccine (2003) 21:4234-4237. MF59 is used as the adjuvant in the FLUAD™ influenza virus trivalent subunit vaccine.

Particularly preferred adjuvants for use in the compositions are submicron oil-in-water emulsions. Preferred submicron oil-in-water emulsions for use herein are squalene/water emulsions optionally containing varying amounts of MTP-PE, such as a submicron oil-in-water emulsion containing 4-5% w/v squalene, 0.25-1.0% w/v Tween 80™ (polyoxyelthylenesorbitan monooleate), and/or 0.25-1.0% Span 85™ (sorbitan trioleate), and, optionally, N-acetylmuramyl-L-alanyl-D-isogluatminyl-L-alanine-2-(1′-2′-dipalmitoyl-sn-glycero-3-huydroxyphosphophoryloxy)-ethylamine (MTP-PE), for example, the submicron oil-in-water emulsion known as “MF59” (International Publication No. WO90/14837; U.S. Pat. Nos. 6,299,884 and 6,451,325, incorporated herein by reference in their entireties; and Ott et al., “MF59—Design and Evaluation of a Safe and Potent Adjuvant for Human Vaccines” in Vaccine Design: The Subunit and Adjuvant Approach (Powell, M. F. and Newman, M. J. eds.) Plenum Press, New York, 1995, pp. 277-296). MF59 contains 4-5% w/v Squalene (e.g. 4.3%), 0.25-0.5% w/v Tween 80™, and 0.5% w/v Span 85™ and optionally contains various amounts of MTP-PE, formulated into submicron particles using a microfluidizer such as Model 110Y microfluidizer (Microfluidics, Newton, Mass.). For example, MTP-PE may be present in an amount of about 0-500 μg/dose, more preferably 0-250 μg/dose and most preferably, 0-100 μg/dose. As used herein, the term “MF59-0” refers to the above submicron oil-in-water emulsion lacking MTP-PE, while the term MF59-MTP denotes a formulation that contains MTP-PE. For instance, “MF59-100” contains 100 μg MTP-PE per dose, and so on. MF69, another submicron oil-in-water emulsion for use herein, contains 4.3% w/v squalene, 0.25% w/v Tween 80™, and 0.75% w/v Span 85™ and optionally MTP-PE. Yet another submicron oil-in-water emulsion is MF75, also known as SAF, containing 10% squalene, 0.4% Tween 80™, 5% pluronic-blocked polymer L121, and thr-MDP, also microfluidized into a submicron emulsion. MF75-MTP denotes an MF75 formulation that includes MTP, such as from 100-400 μg MTP-PE per dose.

Submicron oil-in-water emulsions, methods of making the same and immunostimulating agents, such as muramyl peptides, for use in the compositions, are described in detail in International Publication No. WO90/14837 and U.S. Pat. Nos. 6,299,884 and 6,451,325, incorporated herein by reference in their entireties. Complete Freund's adjuvant (CFA) and incomplete Freund's adjuvant (IFA) may also be used as adjuvants in the invention.

### C. Saponin Formulations

Saponin formulations, may also be used as adjuvants in the invention. Saponins are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs.

Saponin compositions have been purified using High Performance Thin Layer Chromatography (HP-LC) and Reversed Phase High Performance Liquid Chromatography (RP-HPLC). Specific purified fractions using these techniques have been identified, including QS7, QS17, QS18, QS21, QH-A, QH-B and QH-C. Preferably, the saponin is QS21. A method of production of QS21 is disclosed in U.S. Pat. No. 5,057,540. Saponin formulations may also comprise a sterol, such as cholesterol (see WO96/33739).

Combinations of saponins and cholesterols can be used to form unique particles called Immunostimulating Complexes (ISCOMs). ISCOMs typically also include a phospholipid such as phosphatidylethanolamine or phosphatidylcholine. Any known saponin can be used in ISCOMs. Preferably, the ISCOM includes one or more of Quil A, QHA and QHC. ISCOMs are further described in EP0109942, WO96/11711 and WO96/33739. Optionally, the ISCOMS may be devoid of additional detergent. See WO00/07621.

A review of the development of saponin based adjuvants can be found at Barr, et al., “ISCOMs and other saponin based adjuvants”, Advanced Drug Delivery Reviews (1998) 32:247-271. See also Sjolander, et al., “Uptake and adjuvant activity of orally delivered saponin and ISCOM vaccines”, Advanced Drug Delivery Reviews (1998) 32:321-338.

### D. Virosomes and Virus Like Particles (VLPs)

Virosomes and Virus Like Particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1). VLPs are discussed further in WO03/024480, WO03/024481, and Niikura et al., “Chimeric Recombinant Hepatitis E Virus-Like Particles as an Oral Vaccine Vehicle Presenting Foreign Epitopes”, Virology (2002) 293:273-280; Lenz et al., “Papillomavirus-Like Particles Induce Acute Activation of Dendritic Cells”, Journal of Immunology (2001) 5246-5355; Pinto, et al., “Cellular Immune Responses to Human Papillomavirus (HPV)-16 L1 Healthy Volunteers Immunized with Recombinant HPV-16 L1 Virus-Like Particles”, Journal of Infectious Diseases (2003) 188:327-338; and Gerber et al., “Human Papillomavirus Virus-Like Particles Are Efficient Oral Immunogens when Coadministered with Escherichia coli Heat-Labile Entertoxin Mutant R192G or CpG”, Journal of Virology (2001) 75(10):4752-4760. Virosomes are discussed further in, for example, Gluck et al., “New Technology Platforms in the Development of Vaccines for the Future”, Vaccine (2002) 20:B10-B16. Immunopotentiating reconstituted influenza virosomes (IRIV) are used as the subunit antigen delivery system in the intranasal trivalent INFLEXAL™ product {Mischler & Metcalfe (2002) Vaccine 20 Suppl 5:B17-23} and the INFLUVAC PLUS™ product.

### E. Bacterial or Microbial Derivatives

Adjuvants suitable for use in the invention include bacterial or microbial derivatives such as:

(1) Non-Toxic Derivatives of Enterobacterial Lipopolysaccharide (LPS)

Such derivatives include Monophosphoryl lipid A (MPL) and 3-O-deacylated MPL (3dMPL). 3dMPL is a mixture of 3 De-O-acylated monophosphoryl lipid A with 4, 5 or 6 acylated chains. A preferred “small particle” form of 3 De-O-acylated monophosphoryl lipid A is disclosed in EP 0 689 454. Such “small particles” of 3dMPL are small enough to be sterile filtered through a 0.22 micron membrane (see EP 0 689 454). Other non-toxic LPS derivatives include monophosphoryl lipid A mimics, such as aminoalkyl glucosaminide phosphate derivatives e.g. RC-529. See Johnson et al. (1999) Bioorg Med Chem Lett 9:2273-2278.

(2) Lipid A Derivatives

Lipid A derivatives include derivatives of lipid A from Escherichia coli such as OM-174. OM-174 is described for example in Meraldi et al., “OM-174, a New Adjuvant with a Potential for Human Use, Induces a Protective Response with Administered with the Synthetic C-Terminal Fragment 242-310 from the circumsporozoite protein of Plasmodium berghei”, Vaccine (2003) 21:2485-2491; and Pajak, et al., “The Adjuvant OM-174 induces both the migration and maturation of murine dendritic cells in vivo”, Vaccine (2003) 21:836-842.

(3) Immunostimulatory Oligonucleotides

Immunostimulatory oligonucleotides suitable for use as adjuvants in the invention include nucleotide sequences containing a CpG motif (a sequence containing an unmethylated cytosine followed by guanosine and linked by a phosphate bond). Bacterial double stranded RNA or oligonucleotides containing palindromic or poly(dG) sequences have also been shown to be immunostimulatory.

The CpG's can include nucleotide modifications/analogs such as phosphorothioate modifications and can be double-stranded or single-stranded. Optionally, the guanosine may be replaced with an analog such as 2′-deoxy-7-deazaguanosine. See Kandimalla, et al., “Divergent synthetic nucleotide motif recognition pattern: design and development of potent immunomodulatory oligodeoxyribonucleotide agents with distinct cytokine induction profiles”, Nucleic Acids Research (2003) 31(9): 2393-2400; WO02/26757 and WO99/62923 for examples of possible analog substitutions. The adjuvant effect of CpG oligonucleotides is further discussed in Krieg, “CpG motifs: the active ingredient in bacterial extracts?”, Nature Medicine (2003) 9(7): 831-835; McCluskie, et al., “Parenteral and mucosal prime-boost immunization strategies in mice with hepatitis B surface antigen and CpG DNA”, FEMS Immunology and Medical Microbiology (2002) 32:179-185; WO98/40100; U.S. Pat. No. 6,207,646; U.S. Pat. No. 6,239,116 and U.S. Pat. No. 6,429,199.

The CpG sequence may be directed to TLR9, such as the motif GTCGTT or TTCGTT. See Kandimalla, et al., “Toll-like receptor 9: modulation of recognition and cytokine induction by novel synthetic CpG DNAs”, Biochemical Society Transactions (2003) 31 (part 3): 654-658. The CpG sequence may be specific for inducing a Th1 immune response, such as a CpG-A ODN, or it may be more specific for inducing a B cell response, such a CpG-B ODN. CpG-A and CpG-B ODNs are discussed in Blackwell, et al., “CpG-A-Induced Monocyte IFN-gamma-Inducible Protein-10 Production is Regulated by Plasmacytoid Dendritic Cell Derived IFN-alpha”, J. lmmunol. (2003) 170(8):4061-4068; Krieg, “From A to Z on CpG”, TRENDS in Immunology (2002) 23(2): 64-65 and WO01/95935. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, Kandimalla, et al., “Secondary structures in CpG oligonucleotides affect immunostimulatory activity”, BBRC (2003) 306:948-953; Kandimalla, et al., “Toll-like receptor 9: modulation of recognition and cytokine induction by novel synthetic GpG DNAs”, Biochemical Society Transactions (2003) 31(part 3):664-658; Bhagat et al., “CpG penta- and hexadeoxyribonucleotides as potent immunomodulatory agents” BBRC (2003) 300:853-861 and WO03/035836.

Preferably the adjuvant is CpG. Even more preferably, the adjuvant is Alum and an oligonucleotide containing a CpG motif or AlOH and an oligonucleotide containing a CpG motif.

(4) ADP-Ribosylating Toxins and Detoxified Derivatives Thereof.

Bacterial ADP-ribosylating toxins and detoxified derivatives thereof may be used as adjuvants in the invention. Preferably, the protein is derived from E. coli (i.e., E. coli heat labile enterotoxin “LT), cholera (“CT”), or pertussis (“PT”). The use of detoxified ADP-ribosylating toxins as mucosal adjuvants is described in WO95/17211 and as parenteral adjuvants in WO98/42375. Preferably, the adjuvant is a detoxified LT mutant such as LT-K63, LT-R72, and LTR1920. The use of ADP-ribosylating toxins and detoxified derivatives thereof, particularly LT-K63 and LT-R72, as adjuvants can be found in the following references, each of which is specifically incorporated by reference herein in their entirety: Beignon, et al., “The LTR72 Mutant of Heat-Labile Enterotoxin of Escherichia coli Enhances the Ability of Peptide Antigens to Elicit CD4+ T Cells and Secrete Gamma Interferon after Coapplication onto Bare Skin”, Infection and Immunity (2002) 70(6):3012-3019; Pizza, et al., “Mucosal vaccines: non toxic derivatives of LT and CT as mucosal adjuvants”, Vaccine (2001) 19:2534-2541; Pizza, et al., “LTK63 and LTR72, two mucosal adjuvants ready for clinical trials” Int. J. Med. Microbiol (2000) 290(4-5):455-461; Scharton-Kersten et al., “Transcutaneous Immunization with Bacterial ADP-Ribosylating Exotoxins, Subunits and Unrelated Adjuvants”, Infection and Immunity (2000) 68(9):5306-5313; Ryan et al., “Mutants of Escherichia coli Heat-Labile Toxin Act as Effective Mucosal Adjuvants for Nasal Delivery of an Acellular Pertussis Vaccine: Differential Effects of the Nontoxic AB Complex and Enzyme Activity on Th1 and Th2 Cells” Infection and Immunity (1999) 67(12):6270-6280; Partidos et al., “Heat-labile enterotoxin of Escherichia coli and its site-directed mutant LTK63 enhance the proliferative and cytotoxic T-cell responses to intranasally co-immunized synthetic peptides”, Immunol. Lett. (1999) 67(3):209-216; Peppoloni et al., “Mutants of the Escherichia coli heat-labile enterotoxin as safe and strong adjuvants for intranasal delivery of vaccines”, Vaccines (2003) 2(2):285-293; and Pine et al., (2002) “Intranasal immunization with influenza vaccine and a detoxified mutant of heat labile enterotoxin from Escherichia coli (LTK63)” J. Control Release (2002) 85(1-3):263-270. Numerical reference for amino acid substitutions is preferably based on the alignments of the A and B subunits of ADP-ribosylating toxins set forth in Domenighini et al., Mol. Microbiol (1995) 15(6):1165-1167, specifically incorporated herein by reference in its entirety.

Preferably the adjuvant is an ADP-ribosylating toxin and an oligonucleotide containing a CpG motif (see for example, WO 01/34185)

Preferably the adjuvant is a detoxified ADP-ribosylating toxin and an oligonucleotide containing a CpG motif.

Preferably the detoxified ADP-ribosylating toxin is LTK63 or LTK72.

Preferably the adjuvant is LTK63. Preferably the adjuvant is LTK72.

Preferably the adjuvant is LTK63 and an oligonucleotide containing a CpG motif.

Preferably the adjuvant is LTK72 and an oligonucleotide containing a CpG motif.

### F. Bioadhesives and Mucoadhesives

Bioadhesives and mucoadhesives may also be used as adjuvants in the invention. Suitable bioadhesives include esterified hyaluronic acid microspheres (Singh et al. (2001) J. Cont. Rele. 70:267-276) or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention. E.g. WO99/27960.

### G. Microparticles

Microparticles may also be used as adjuvants in the invention. Microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

### H. Liposomes

Examples of liposome formulations suitable for use as adjuvants are described in U.S. Pat. No. 6,090,406, U.S. Pat. No. 5,916,588, and EP 0 626 169.

### L Polyoxyethylene Ether and Polyoxyethylene Ester Formulations

Adjuvants suitable for use in the invention include polyoxyethylene ethers and polyoxyethylene esters. WO99/52549. Such formulations further include polyoxyethylene sorbitan ester surfactants in combination with an octoxynol (WO01/21207) as well as polyoxyethylene alkyl ethers or ester surfactants in combination with at least one additional non-ionic surfactant such as an octoxynol (WO01/21152).

Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

### J. Polyphosphazene (PCPP)

PCPP formulations are described, for example, in Andrianov et al., “Preparation of hydrogel microspheres by coacervation of aqueous polyphophazene solutions”, Biomaterials (1998) 19(1-3):109-115 and Payne et al., “Protein Release from Polyphosphazene Matrices”, Adv. Drug. Delivery Review (1998) 31(3):185-196.

### K. Muramyl Peptides

Examples of muramyl peptides suitable for use as adjuvants in the invention include N-acetyl-muramyl-L-threonyl-D-isoglutamine(thr-MDP), N-acetyl-normuramyl-1-alanyl-d-isoglutamine(nor-MDP), and N-acetylmuramyl-1-alanyl-d-isoglutaminyl-1-alanine-2-(1′-2′-dipalmitoyl-sn-glycero-3-hydroxyphosphoryloxy)-ethylamine MTP-PE).

### L. Imidazoquinolone Compounds

Examples of imidazoquinolone compounds suitable for use adjuvants in the invention include Imiquamod and its homologues, described further in Stanley, “Imiquimod and the imidazoquinolones: mechanism of action and therapeutic potential” Clin Exp Dermatol (2002) 27(7):571-577 and Jones, “Resiquimod 3M”, Curr Opin Investig Drugs (2003) 4(2):214-218.

The invention may also comprise combinations of aspects of one or more of the adjuvants identified above. For example, the following adjuvant compositions may be used in the invention:
(1) a saponin and an oil-in-water emulsion (WO99/11241);
(2) a saponin (e.g., QS21)+a non-toxic LPS derivative (e.g. 3dMPL) (see WO94/00153);
(3) a saponin (e.g., QS21)+a non-toxic LPS derivative (e.g. 3dMPL)+a cholesterol;
(4) a saponin (e.g. QS21)+3dMPL+IL-12 (optionally+a sterol) (WO98/57659);
(5) combinations of 3dMPL with, for example, QS21 and/or oil-in-water emulsions (See European patent applications 0835318, 0735898 and 0761231);
(6) SAF, containing 10% Squalane, 0.4% Tween 80, 5% pluronic-block polymer L121, and thr-MDP, either microfluidized into a submicron emulsion or vortexed to generate a larger particle size emulsion.
(7) Ribi™ adjuvant system (RAS), (Ribi Immunochem) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™);
(8) one or more mineral salts (such as an aluminum salt)+a non-toxic derivative of LPS (such as 3dPML); and
(9) one or more mineral salts (such as an aluminum salt)+an immunostimulatory oligonucleotide (such as a nucleotide sequence including a CpG motif).

Aluminum salts and MF59 are preferred adjuvants for use with injectable influenza vaccines. Bacterial toxins and bioadhesives are preferred adjuvants for use with mucosally-delivered vaccines, such as nasal vaccines.

### M. Human Immunomodulators

Human immunomodulators suitable for use as adjuvants in the invention include cytokines, such as interleukins (e.g. IL-1, IL-2, IL-4, IL-5, IL-6, IL-7, IL-12, etc.), interferons (e.g. interferon-γ), macrophage colony stimulating factor, and tumor necrosis factor.

### Further Antigens

The compositions of the invention may further comprise antigen derived from one or more sexually transmitted diseases in addition to Chlamydia trachomatis. Preferably the antigen is derived from one or more of the following sexually transmitted diseases: N. gonorrhoeae (See e.g. WO99/24578, WO99/36544, WO99/57280, WO02/079243); human papilloma virus; Treponema pallidum; herpes simplex virus (HSV-1 or HSV-2); HIV (HIV-1 or HIV-2); and Haemophilus ducreyi.

A preferred composition comprises: (1) at least t of the Chlamydia trachomatis antigens from either the first antigen group or the second antigen group, where t is 2, 3, 4, 5, 6, 7, 8, 9, 10, 11, 12 or 13, preferably t is five; (2) one or more antigens from another sexually transmitted disease. Preferably, the sexually transmitted disease is selected from the group consisting of herpes simplex virus, preferably HSV-1 and/or HSV-2; human papillomavirus; N. gonorrhoeae; Treponema pallidum; and Haemophilus ducreyi. These compositions can thus provide protection against the following sexually-transmitted diseases: chlamydia, genital herpes, genital warts, gonorrhoea, syphilis and chancroid (See, WO00/15255).

Antigens associated with or derived from N. gonorrhoeae may include, for example, a Por (or porin) protein, such as PorB (see Zhu et al., Vaccine (2004) 22:660-669), a transferring binding protein, such as TbpA and TbpB (See Price et al., Infection and Immunity (2004) 71(1):277-283), a opacity protein (such as Opa), a reduction-modifiable protein (Rmp), and outer membrane vesicle (OMV) preparations (see Plante et al., J Infectious Disease (2000) 182:848-855).

Antigens associated with or derived from human papillomavirus (HPV) may include, for example, one or more of E1-E7, L1, L2, and fusions thereof. Preferably, the compositions of the invention may include a virus-like particle (VLP) comprising the L1 major capsid protein. Preferably the HPV antigens are protective against one or more of HPV serotypes 6, 11, 16 and 18.

Where a saccharide or carbohydrate antigen is used, it is preferably conjugated to a carrier protein in order to enhance immunogenicity (See e.g. Ramsay et al. (2001) Lancet 357(9251):195-196; Lindberg (1999) Vaccine 17 Suppl 2:S28-36; Buttery & Moxon (2000) J R Coll Physicians Lond 34:163-168; Ahmad & Chapnick (1999) Infect Dis Clin North Am 13:113-133, viiGoldblatt (1998) J. Med. Microbiol. 47:563-567; European patent 0 477 508; U.S. Pat. No. 5,306,492 International patent application WO98/42721 Conjugate Vaccines (eds. Cruse et al.) ISBN 3805549326, particularly vol. 10:48-114 Hermanson (1996) Bioconjugate Techniques ISBN: 0123423368 or 012342335X). Preferred carrier proteins are bacterial toxins or toxoids, such as diphtheria or tetanus toxoids. The CRM₁₉₇ diphtheria toxoid is particularly preferred (See Research Disclosure, 453077 (January 2002). Other carrier polypeptides include the N. meningitidis outer membrane protein (See EP-A-0372501), synthetic peptides (See EP-A-0378881 and EP-A-0427347), heat shock proteins (See WO93/17712 and WO94/03208), pertussis proteins (See WO98/58668 and EP-A-0471177), protein D from H. influenzae (See WO00/56360), cytokines (See WO91/01146), lymphokines, hormones, growth factors, toxin A or B from C. difficile (See WO00/61761), iron-uptake proteins (See WO01/72337), etc. Where a mixture comprises capsular saccharides from both serogroups A and C, it may be preferred that the ratio (w/w) of MenA saccharide:MenC saccharide is greater than 1 (e.g. 2:1, 3:1, 4:1, 5:1, 10:1 or higher). Different saccharides can be conjugated to the same or different type of carrier protein. Any suitable conjugation reaction can be used, with any suitable linker where necessary.

Toxic protein antigens may be detoxified where necessary e.g. detoxification of pertussis toxin by chemical and/or genetic means.

Where a diphtheria antigen is included in the composition it is preferred also to include tetanus antigen and pertussis antigens. Similarly, where a tetanus antigen is included it is preferred also to include diphtheria and pertussis antigens. Similarly, where a pertussis antigen is included it is preferred also to include diphtheria and tetanus antigens.

Antigens in the composition will typically be present at a concentration of at least 1 μg/ml each.

In general, the concentration of any given antigen will be sufficient to elicit an immune response against that antigen.

As an alternative to using protein antigens in the composition of the invention, nucleic acid encoding the antigen may be used (See e.g. Robinson & Torres (1997) Seminars in Immunology 9:271-283; Donnelly et al. (1997) Annu Rev Immunol 15:617-648 Scott-Taylor & Dalgleish (2000) Expert Opin Investig Drugs 9:471-480 Apostolopoulos & Plebanski (2000) Curr Opin Mol Ther 2:441-447 Ilan (1999) Curr Opin Mol Ther 1:116-120 Dubensky et al. (2000) Mol Med 6:723-732; Robinson & Pertmer (2000) Adv Virus Res 55:1-74 Donnelly et al. (2000) Am J Respir Crit Care Med 162(4 Pt 2):S190-193 Davis (1999) Mt. Sinai J. Med. 66:84-90). Protein components of the compositions of the invention may thus be replaced by nucleic acid (preferably DNA e.g. in the form of a plasmid) that encodes the protein.

### Definitions

The term “comprising” means “including” as well as “consisting” e.g. a composition “comprising” X may consist exclusively of X or may include something additional e.g. X+Y.

The term “about” in relation to a numerical value x means, for example, x±10%.

References to a percentage sequence identity between two amino acid sequences means that, when aligned, that percentage of amino acids are the same in comparing the two sequences. This alignment and the percent homology or sequence identity can be determined using software programs known in the art, for example those described in section 7.7.18 of Current Protocols in Molecular Biology (F. M. Ausubel et al., eds., 1987) Supplement 30. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in Smith & Waterman (1981) Adv. Appl. Math. 2: 482-489

### Examples

The present invention will be defined only by way of example. It will be understood that the invention has been described by way of example only and modifications may be made whilst remaining within the scope and spirit of the invention. Tables 1(a) and 1(b), below, summarize characterisation data of the CT antigens of the invention. These tables also include data which will be further explained in the examples which follow.

The following columns are set forth in Table 1(a): Gene identification number (Gene ID), Protein ID and the corresponding Current Annotation were retrieved from the D/UW-3/CX genome filed in GenBank (accession number AE001273). Fusion Type: Indicates whether the data was generated from a His or GST fusion peptide (or both). Theoretical Molecular Weight represents the molecular mass (in kilodaltons) which were calculated for predicted mature forms of the referenced protein. Antiserum: Western blot Analysis (WB profile) summarizes the western blot results obtained by probing total EB proteins with antisera against the respective recombinant CT proteins. The number in brackets refers to panel number in FIG. 2. WB results are classified as follows: C indicates Consistent (i.e., the predominant band observed is consistent with the expected molecular weight; additional minor bands may also be present); PC indicates Partially Consistent (i.e., a band of expected molecular weight is present together with additional bands of higher molecular weight or greater intensity); NC represents Nonconsistent (i.e., the detected bands do not correspond to the expected molecular weight); N represents Negative (i.e., no profile obtained). Antiserum: FACS Assay (KS score) includes the results of FACS analysis, expressed as K-S scores. The serum titers giving 50% neutralization of infectivity for the 9 C. trachomatis recombinant antigens described in the text (PepA, ArtJ, DnaK, CT398, CT547, Enolase, MOMP, OmpH-like, Atos). Each titer was assessed in 3 separate experiments (SEM values shown). Antiserum: Neutralizing Titre (reciprocal) represents neutralizing antibody titres for the respective CT antigens. The results are as follows: PepA (CT045) 1:100; ArtJ (CT381) 1:370; DnaK (CT396) 1:230; Hypothetical (CT398) 1:540; Hypothetical (CT547) 1:40; Enolase (CT587) 1:180; MOMP (CT681) 1:160; OmpH-like (CT242) 1:190; AtoS (CT467) 1:500. All of the proteins that showed a K-S score higher than 8.0 have been listed as FACS-positive. Antigen: Reported 2DE/MALDI-TOF detection are depicted as yes/no/? (=not determined) results in the last column of the Table.

TABLE 1(a)
Characterisation of Chlamydia trachomatis (CT) expressed proteins

| | | | | | | | | Antigen: |
|---|---|---|---|---|---|---|---|---|
| | | | | | | | | Reported |
| | | | | | | | Antiserum: | 2DE/ |
| | | | | | | Antiserum: | Neutralizing | MALDI- |
| | Protein | | | Theoretical | Antiserum: | FACS assay (K [symbol] | titre | TOF |
| Gene ID | ID | Current annotation | Fusion type | MWt (kDa) | WB analysis | S score) | (reciprocal) | detection |
| CT045 | PepA | pep A (Leucyl Aminopeptidase A) | HIS | 54.0 | C | 16.81 | 100 | Yes |
| CT381 | ArtJ | artJ (Arginine Binding Protein) | HIS | 26.0 | C | 32.54 | 370 | No |
| CT396 | DnaK | dnaK (HSP-70 heat shock protein) | HIS | 70.6 | C | 34.50 | 230 | Yes |
| CT398 | CT398 | Hypothetical protein | His&GST | 29.4 | C | 31.24 | 540 | Yes |
| CT547 | CT547 | Hypothetical protein | HIS | 32.6 | PC | 28.21 | 40 | No |
| CT587 | Enolase | eno (Enolase) | HIS | 45.3 | C | 20.85 | 180 | Yes |
| CT681 | MOMP | ompA (Major Outer Membrane | HIS | 40.1 | C | 34.66 | 160 | Yes |
| | | Protein) | | | | | | |
| CT242 | OmpH | ompH-Like Outer Membrane | HIS | 15.8 | C | <8 | 190 | No |
| | | Protein | | | | | | |
| CT467 | AToS | atoS (2-component sensor histidine | GST | 39.8 | N | <8 | 500 | No |
| | | kinase) | | | | | | |
| CT043 | CT043 | hypothetical<<Cpn0387 | GST | 18.4 | ? | 27.53 | ? | ? |
| CT050 | CT050 | Hypothetical protein | GST | 56.6 | C (1) | 20.68 | <30 | No |
| CT082 | CT082 | Hypothetical protein. | GST | 59.4 | C (2) | 25.63 | <30 | Yes |
| CT089 | LcrE | lcrE (Low Calcium response E) | HIS | 43.0 | C (3) | 12.59 | <30 | No |
| CT128 | Adk | adk (adenylate kinase) | GST | 27.6 | C (4) | 16.00 | <30 | No |
| CT153 | CT153 | hpothetical >Cpn0176 (6445) | GST | 90.8 | ? | 13.33 | ? | ? |
| | | <<MAC/perform domain | | | | | | |
| CT157 | CT157 | Phospholipase D Superfamily | GST | 45.2 | C (5) | 19.77 | <30 | No |
| CT165 | CT165 | Hypothetical protein | GST | 16.8 | C (6) | 10.46 | <30 | No |
| CT262 | CT262 | hypothetical > Cpn0411 | His-ib | 28.7 | ? | 19.31 | ? | ? |
| CT266 | CT266 | Hypothetical protein | HIS | 43.9 | PC (7) | 21.29 | <30 | No |
| | | >Cpn0415 (6696) | | | | | | |
| CT276 | CT276 | hypothetical (acidic) > Cpn0425 | GST | 21.3 | ? | 19.85 | ? | ? |
| | | (6706) | | | | | | |
| CT296 | dcrA | hypothetical divalent cation | GST | 17.9 | ? | 17.70 | ? | ? |
| | | dependent regulator (Raulston) | | | | | | |
| CT316 | L7/L12 | rl7 (Ribosomal protein L7/L12) | HIS | 13.4 | C (8) | 9.68 | <30 | Yes |
| CT372 | CT372 | hypothetical (basic) | HIS | 49.3 | ? | 24.77 | ? | ? |
| CT443 | OmcB | omcB (60 kDa Cysteine-Rich OMP) | HIS | 56.2 | C (9) | 21.28 | <30 | Yes |
| CT444 | OmcA | omcA (9 kDa Cysteine-Rich OMP) | GST | 9.0 | PC (10) | 15.00 | <30 | No |
| CT456 | CT456 | Hypothetical protein | GST | 97.6 | N (11) | 10.90 | <30 | Yes |
| CT480 | oppA | oligopeptide binding protein (1 of 5 | pHis&pGST | 58.8 | ? | 27.45/9.48 | ? | ? |
| | | genes) | | | | | | |
| CT541 | Mip- | mip (FKBP-type cis-trans | GST | 24.5 | C (12) | 9.94 | <30 | Yes |
| | like | Isomerase) | | | | | | |
| CT548 | CT548 | hypothetical | GST | ? | ? | 14.78 | ? | ? |
| CT559 | YscJ | yscJ (Yop proteins translocation | HIS | 33.3 | C (13) | 23.21 | <30 | No |
| | | lipoprotein J) | | | | | | |
| CT600 | Pal | pal (Peptidoglycan-Associated | HIS | 19.1 | C (14) | 10.46 | <30 | No |
| | | Lipoprotein) | | | | | | |
| CT623 | CT623 | CHLPN 76 kDa Homolog | GST | 45.6 | C (15) | 15.89 | <30 | No |
| CT635 | CT635 | hypothetical | His&GST | ? | ? | 11.62/11.52 | ? | ? |
| CT671 | CT671 | hypothetical | his | ? | ? | 9.29 | ? | ? |
| CT713 | PorB | porB (Outer Membrane Protein | HIS | 34.4 | C (16) | 25.82 | <30 | Yes |
| | | Analog) | | | | | | |
| CT823 | HtrA | htrA (DO serine protease) | HIS | 51.4 | PC (17) | 26.62 | <30 | Yes |
| CT859 | CT859 | metalloprotese | his&GST | ? | ? | 10.91/9.46 | ? | ? |
| CT412 | pmpA | OM protein A | His | 105.6 | ? | 10.92 | ? | ? |
| CT414 | PmpC | pmpC (Putative outer membrane | GST | 184.9 | C (18) | 9.03 | <30 | No |
| | | protein C) | | | | | | |
| CT812 | PmpD | pmpD (Putative Outer Membrane | GST | 157.6 | N (19) | 10.43 | <30 | Yes |
| | | Protein D) | | | | | | |
| CT869 | PmpE | pmpE (Putative Outer Membrane | HIS | 102.7 | N (20) | 15.28 | <30 | No |
| | | Protein E) | | | | | | |

[symbol]

indicates data missing or illegible when filed

Similar columns are represented in Table 1(b). In this table, the In-vitro Neutralizing Activity column, indicates either neg (negative) or ND (not determined).

TABLE 1(b)
Characterisation of Expressed Chlamydia trachomatis (CT) Proteins cont

| | | | | | | In-vitro |
|---|---|---|---|---|---|---|
| | | | Molecular | Western | K-S | neut |
| Gene ID | Gene Annotation | Fusion Type | Mass (kDa) | Blot (WB) | Score | activity |
| CT016 | Hypothetical | HIS | 26.63 | Neg | 17.94 | neg |
| CT017 | Hypothetical | HIS | 47.79 | Neg | 12.18 | neg |
| CT043 | Hypothetical | HIS | 18.38 | Consistent | 27.53 | neg |
| CT082 | Hypothetical | HIS | 59 | Partly C | 15.89 | neg |
| CT548 | Hypothetical | GST | 21.9 | C | 14.78 | neg |
| CT153 | Hypothetical | GST | 90.86 | C | 13.33 | neg |
| CT262 | Hypothetical | HIS | 28.81 | Neg | 19.31 | neg |
| CT276 | Hypothetical | GST | 21.37 | Not C | 19.85 | neg |
| CT296 | Hypothetical | GST | 17.98 | Neg | 17.70 | neg |
| CT372 | Hypothetical | HIS | 49.00 | Partly C | 24.77 | neg |
| CT398 | Hypothetical | GST | | | 27.03 | neg |
| CT398 | Hypothetical | HIS | | | 22.96 | neg |
| CT548 | Hypothetical | GST | | | 14.78 | neg |
| CT043 | Hypothetical | HIS | | | 27.53 | neg |
| CT635 | Hypothetical | GST | 16.77 | Neg | 11.52 | ND |
| CT635 | Hypothetical | HIS | 16.77 | Neg | 11.62 | ND |
| CT671 | Hypothetical | HIS | 31 | Neg | 20.91 | ND |
| CT671 | Hypothetical | GST | 31 | Neg | 18.07 | ND |
| CT089 | Low Calcium | GST | 44 | C | 11.9 | neg |
| | Response Element | | | | | |
| | (LcrE) | | | | | |
| CT812 | PmpD | GST | 168 | Not C | 23.48 | neg |
| CT412 | Putative Outer | HIS | 107 | Not C | 10.92 | neg |
| | Membrane Protein A | | | | | |
| CT480 | Oligopeptide | GST | 79.89 | C | 9.48 | neg |
| | Binding | | | | | |
| | Lipoprotein | | | | | |
| CT480 | Oligopeptide | HIS | 79.89 | C | 27.45 | neg |
| | Binding | | | | | |
| | Lipoprotein | | | | | |
| CT859 | Metalloprotease | GST | 34.21 | C | 9.46 | ND |
| CT859 | Metalloprotease | HIS | 34.21 | C | 10.91 | neg |
| CT869 | PmpE | GST | 106 | PC | 30.67 | neg |
| CT053 | | | | | | ND |

### Example 1

### Western Blot, FACS and In Vitro Neutralization Assay and Analysis of CT Antigens, as Shown in Table 1(a)

The Western Blot, FACS and In Vitro Neutralization assays and analysis of Tables 1(a) and 1(b) are further discussed in this Example. Preparation of the materials and details of these assays are set forth below.

Preparation of C. trachomatis EBs and chromosomal DNA: C. trachomatis GO/96, a clinical isolate of C. trachomatis serotype D from a patient with non-gonococcal urethritis at the Sant'Orsola Polyclinic, Bologna, Italy, was grown in LLC-MK2 cell cultures (ATCC CCL-7). EBs were harvested 48 h after infection and purified by gradient centrifugation as described previously (See Schachter, J., and P. B. Wyrick. 1994. Methods Enzymol. 236:377-390). Purified chlamydiae were resuspended in sucrose-phosphate transport buffer and stored at −80° C. until use. When required, prior to storage EB infectivity was heat inactivated by 3 h of incubation at 56° C. Chromosomal DNA was prepared from gradient-purified EBs by lysing the cells overnight at 37° C. with 10 mM Tris-HCl, 150 mM NaCl, 3 mM EDTA, 0.6% SDS, 100 μg of proteinase K/ml, sequential extraction with phenol, phenol-chloroform, and chloroform, alcohol precipitation and resuspension in TE buffer, pH 8.

In silico analyses: All the 894 protein coding genes and the corresponding peptide sequences encoded by the C. trachomatis genome UW-3/Cx (Stephens et al., 1998. Science 282: 754-9) were retrieved from the National Center for Biotechnology Information web site (http://www.ncbi.nlm.nih.gov/). Putative surface exposed proteins were selected primarily on the basis of GenBank annotation and sequence similarity to proteins known to be secreted or surface-associated. Sequences annotated as hypothetical, which typically lack significant homologies to well characterized proteins, were analyzed for the presence of leader peptide and/or transmembrane regions with PSORT algorithm (Gardy et al., Nucleic Acids Res. 2003 Jul. 1; 31(13):3613-7). Following these criteria, a set of 158 peptides were selected for expression and in vitro screening.

Cloning and expression of recombinant proteins: Selected ORFs from the C. trachomatis UW-3/Cx genome (Stephens et al., supra) were cloned into plasmid expression vectors so as to obtain two kinds of recombinant proteins: (i) proteins with a hexa-histidine tag at the C terminus (ct-His), and (ii) proteins fused with both glutathione S-transferase (GST) at their N terminus and a hexa-histidine tag at their C terminus (Gst-ct) as described in (Montigiani, et al., 2002. Infect Immun 70:368-79). Escherichia coli BL21 and BL21(DE3) (Novagen) were the recipient of pET21b-derived recombinant plasmids and pGEX-derived plasmids respectively. PCR primers were designed so as to amplify genes without the signal peptide coding sequence. When a signal peptide or processing site was not clearly predictable, the ORF sequence was cloned in its full-length form. Recombinant clones were grown in Luria-Bertani medium (500 ml) containing 100 ug of ampicillin/ml and grown at 37° C. until an optical density at 600 nm (OD600) of 0.5 was reached. Expression of recombinant proteins was then induced by adding 1 mM isopropyl-D-thiogalactopyranoside (IPTG). Three hours after IPTG induction, cells were collected by centrifugation at 6000×g for 20 min. at 4° C. Before protein purification, aliquots of the cell pellets (corresponding to an OD600 of 0.1) were resuspended in sample loading buffer (60 mM Tris-HCl [pH 6.8], 5% [wt/vol] SDS, 10% [vol/vol] glycerol, 0.1% [wt/vol] bromophenol blue, 100 mM dithiothreitol [DTT]), boiled for 5 min, and analyzed by SDS-polyacrylamide gel electrophoresis (SDS-PAGE).

Purification of recombinant proteins. The cell pellets obtained from centrifugation of 500 ml induced recombinant E. coli cultures were suspended with 10 ml B-PER™ (Bacterial-Protein Extraction Reagent, Pierce), 1 mM MgCl2, 100 Kunits units DNAse I (Sigma), and 1 mg/ml lysozime (Sigma). After 30 min at room temperature under gentle shaking the lysate was clarified by centrifugation at 30.000 g for 30 min at 4° C. and the supernatant (soluble proteins) was separated from the pellet (debris, insoluble proteins and inclusion bodies).

Soluble His-tagged proteins were purified by an immobilized metal affinity chromatography (IMAC) using 1 ml mini-columns of Ni-activated Chelating Sepharose Fast Flow (Amersham). After loading the column was washed with 20 mM Imidazole and the remaining proteins were eluted by one step elution using 250 mM Imidazole buffer, 50 mM phosphate, 300 mM NaCl, pH 8.0.

Insoluble His-tagged proteins were purified by suspending the pellet, coming from centrifugation of B-PER lysate, in 50 mM TRIS-HCl, 1 mM TCEP (Tris(2-carboxyethyl)-phosphine hydrochloride, Pierce) and 6M guanidine hydrochloride, pH 8.5, and performing an IMAC in denaturing conditions of the clarified solubilized proteins. Briefly: the resuspended material was centrifuged at 30.000 g for 30 min and the supernatant was loaded on 1 ml minicolumns of Ni-activated Chelating Sepharose Fast Flow (Pharmacia) equilibrated with 50 mM TRIS-HCl, 1 mM TCEP, 6M guanidine hydrochloride, pH 8.5. The column was washed with 50 mM TRIS-HCl buffer, 1 mM TCEP, 6M urea, 20 mM imidazole, pH 8.5. Recombinant proteins were then eluted with the same buffer containing 250 mM imidazole.

The soluble GST-fusion proteins were purified by subjecting the B-PER soluble lysate to glutathione affinity purification using 0.5 ml mini-columns of Glutathione-Sepharose 4B resin (Amersham) equilibrated with 10 ml PBS, pH 7.4. After column washing with equilibrium buffer the proteins were eluted with 50 mM TRIS buffer, 10 mM reduced glutathione, pH 8.0.

Protein concentration was determined using the Bradford method.

As the Examples demonstrate, in some embodiments, a HIS tagged protein was used whereas in other embodiments a GST tagged protein was used. In other instances, combinations of HIS tagged or GST tagged proteins were used. Preferably the immunogenic compositions comprise one or more HIS tagged proteins.

Eluted protein fractions were analyzed by SDS-Page and purified proteins were stored at −20° C. after addition of 2 mM Dithiothreitol (Sigma) and 40% glycerol.

Preparation of mouse antisera: Groups of four 5- to 6-week-old CD1 female mice (Charles River, Como, Italy) were immunized intraperitoneally at days 1, 15, and 28 with 20 ug of purified recombinant protein in Freund's adjuvant. Pre-immune and immune sera were prepared from blood samples collected on days 0 and 43 respectively and pooled before use. In order to reduce the amount of antibodies possibly elicited by contaminating E. coli antigens, the immune sera were incubated overnight at 4° C. with nitrocellulose strips adsorbed with an E. coli BL21 total protein extract.

Immunological assays: For Western blot analysis, total proteins from purified C. trachomatis GO/96 serotype D EBs (2 ug per lane) were separated by SDS-PAGE and electroblotted onto nitrocellulose membranes. After 30 min. of saturation with PBS-dried skimmed milk (5% w/v) membranes were incubated overnight with preimmune and immune sera (standard dilution 1:400) and then washed 3× with phosphate-buffered saline (PBS)-Tween 20 (0.1% v/v). Following a 1 hour incubation with a peroxidase-conjugated anti-mouse antibody (final dilution 1:5,000 Amersham;) and washing with PBS-Tween, blots were developed using an Opti-4CN Substrate Kit (Bio-Rad).

Flow cytometry assays: Analyses were performed essentially as previously described (See Montigiani et al., supra). Gradient purified, heat-inactivated GO/96 serotype D EBs (2×105 cells) from C. trachomatis resuspended in phosphate-saline buffer (PBS), 0.1% bovine serum albumin (BSA), were incubated for 30 min. at 4° C. with the specific mouse antisera (standard dilution 1:400). After centrifugation and washing with 200 μl of PBS-0.1% BSA, the samples were incubated for 30 minutes at 4° C. with Goat Anti-Mouse IgG, F(ab)′2-specific, conjugated with R-Phycoerythrin (Jackson Immunoresearch Laboratories Inc.). The samples were washed with PBS-0.1% BSA, resuspended in 150 μl of PBS-0.1% BSA and analysed by Flow Cytometry using a FACSCalibur apparatus (Becton Dickinson, Mountain View, Calif.). Control samples were similarly prepared. Positive control antibodies were: i), a commercial anti-C. pneumoniae specific monoclonal antibody (Argene Biosoft, Varilhes, France) and, ii), a mouse polyclonal serum prepared by immunizing mice with gradient purified C. trachomatis EBs.

Background control sera were obtained from mice immunized with the purified GST or HIS peptide used in the fusion constructs (GST control, HIS control). FACS data were analysed using the Cell Quest Software (Becton Dickinson, Mountain View, Calif.). The significance of the FACS assay data has been elaborated by calculating the Kolmogorov-Smirnov statistic (K-S score.) (See Young, I. T. 1977. J Histochem Cytochem 25:935-41). The K-S statistic allows determining the significance of the difference between two overlaid histograms representing the FACS profiles of a testing protein antiserum and its relative control. All the proteins that showed a K-S score higher than 8.0 have been listed as FACS positive, being the difference between the two histograms statistically significant (p<0.05). The D/s(n) values (an index of dissimilarity between the two curves) are reported as “K-S score” in Tables 1(a) and 1(b).

In vitro neutralization assays: In vitro neutralization assays were performed on LLC-MK2 (Rhesus monkey kidney) epithelial cell cultures. Serial four-fold dilutions of mouse immune and corresponding preimmune sera were prepared in sucrose-phosphate-glutamic acid buffer (SPG). Mouse polyclonal sera to whole EBs were used as positive control of neutralization, whereas SPG buffer alone was used as negative control of neutralization (control of infection). Purified infectious EBs from C. trachomatis GO/96 serotype D were diluted in SPG buffer to contain 3×10⁵ IFU/ml, and 10 ul of EBs suspension were added to each serum dilution in a final volume of 100 ul. Antibody-EB interaction was allowed to proceed for 30 min at 37° C. on a slowly rocking platform. The 100 ul of reaction mix from each sample was used to inoculate PBS-washed LLC-MK2 confluent monolayers (in triplicate for each serum dilution), in a 96-well tissue culture plate, and centrifuged at 805×g for 1 hour at 37° C. After centrifugation Eagle's minimal essential medium containing Earle's salts, 20% fetal bovine serum and 1 ug/ml cycloheximide was added. Infected cultures were incubated at 37° C. in 5% CO₂ for 72 hours. The monolayers were fixed with methanol and the chlamydial inclusions were detected by staining with a mouse anti-Chlamydia fluorescein-conjugated monoclonal antibody (Merifluor Chlamydia, Meridian Diagnostics, Inc.) and quantified by counting 5 fields per well at a magnification of 40×. The inhibition of infectivity due to EBs interaction with the immune sera was calculated as percentage reduction in mean IFU number as compared to the SPG (buffer only)/EBs control. In this calculation the IFU counts obtained with immune sera were corrected for background inhibition of infection due to the corresponding pre-immune mouse serum. According to common practice, the sera were considered as “neutralizing” if they could cause a 50% or greater reduction in infectivity. The corresponding neutralizing titer was defined as the serum dilution at which a 50% reduction of infectivity was observed. Experimental variability was evaluated by calculating the standard error of measurement (SEM), from three titration experiments for each recombinant antigen, as shown in FIG. 2.

Results of the Western Blot, FACS and In Vitro Neutralization assays and analysis are depicted in Tables 1(a) and 1(b) and are further discussed below.

In silico selection: The genomic ORFs to be expressed and submitted to functional screenings were selected on the basis of in silico analyses and literature searches, using bioinformatics tools and criteria similar to those described in a previous similar study on C. pneumoniae (Montigiani, et al., 2002). Essentially, we searched the genome of C. trachomatis serovar D for ORF's encoding proteins likely to be located on the surface of EBs. In order to maximize the chances of identifying bacterial surface proteins we initially selected C. trachomatis proteins having a significant sequence similarity to proteins found to be surface exposed in C. pneumoniae as previously reported (Montigiani, et al., 2002). A second step search was based essentially on the presence of a recognizable leader peptide (mostly as detected by the PSORT software), predicted transmembrane regions, and/or remote sequence similarities to surface proteins of other gram-negative bacteria detected with PSI-Blast runs against the non-redundant GenBank protein database. A third criterion was the addition to the panel of proteins described as immunogenic in animal models and humans. Using this procedure we selected a total of 158 ORFs, 114 of which had at least 40% of identity to proteins of C. pneumoniae, while 44 remained below such threshold and were considered as C. trachomatis specific.

Antigen cloning and expression: The 158 ORFs were amplified by PCRs and cloned in two different E. coli expression vectors in order to obtain S each antigen as GST and/or His-tag fusion protein. Considering that the presence of an N-terminal signal peptide could have induced a possible targeting of the recombinant protein toward the E. coli cytoplasmic membrane, the N-terminal signal peptide nucleotide sequence was excluded from the expression construct. By the analysis of the ORFs expression we found that 94% of the selected genes could be expressed and 87% of them (corresponding to 137 different ORFs) could also be purified to recombinant fusion proteins that could be used as antigens for mice immunization. In total, 259 recombinant C. trachomatis fusion proteins, deriving from the 137 different genes cloned, were obtained and analysed for their quality in order to be used as antigens for mice immunization. Mice were immunized with 201 recombinant C. trachomatis fusion proteins to produce mouse sera that have been analysed for their capability to recognize surface exposed proteins on C. trachomatis EBs and their capability of interfering with the process of in vitro infection of epithelial cell culture.

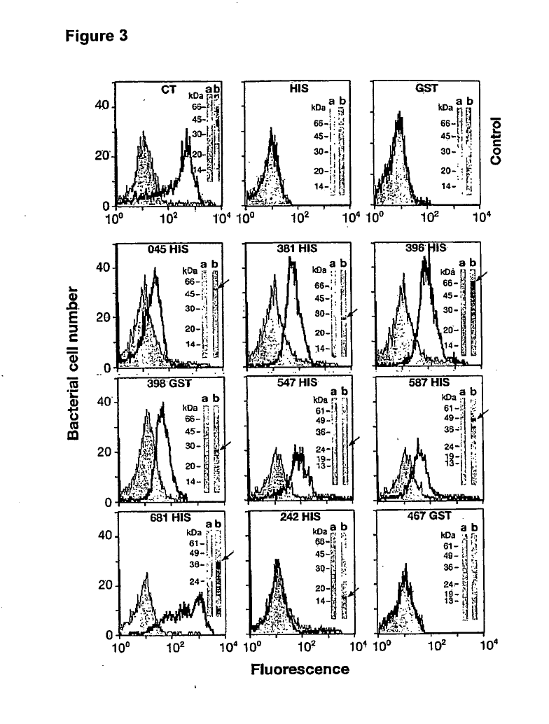

Identification of surface exposed proteins by flow cytometry: Mice were immunized with 201 recombinant C. trachomatis fusion proteins to produce mouse sera that have been analysed both for their capability to recognize surface exposed proteins on C. trachomatis EBs and their capability of interfering with the process of in vitro infection of epithelial cell culture. Immunofluorescent staining of C. trachomatis EBs and flow cytometric analysis have been used to investigate the capability of mouse sera, obtained by immunization with a panel of 137 different C. trachomatis recombinant antigens, to recognize possibly surface exposed proteins. We had previously shown that flow cytometry can be a very useful tool to detect antibody binding to the surface of chlamydial EBs, by identifying a new panel of C. pneumoniae surface exposed proteins. Although C. trachomatis serovar L and E had already been analyzed by flow cytometry (See Waldman, et al., (1987) Cytometry 8, 55-59; and Taraktchoglou, et al., (2001). Infect Immun 69, 968-76), we first verified if this method could also be applied to C. trachomatis serovar D EBs analysis, by setting up a series of positive and negative controls. As shown in FIG. 3, Panel A, a mouse polyclonal serum obtained by immunizing mice with purified whole C. trachomatis serovar D EBs, can significantly shift the flow cytometric profile of the bacterial cell population, as compared to a negative, pre-immune serum. As a positive control we also used a commercial anti-MOMP C. trachomatis specific monoclonal antibody (Argene), which gave a similar result as the polyclonal serum (data not shown). We also set up a series of negative controls, to exclude possible cross-reactions between mouse sera and the chlamydial cell surface. In particular sera obtained by immunizing mice with the protein fraction eluted from the Ni columns loaded with a BL21(pET21b+) protein extract (His control, FIG. 3, Panel 2) and with GST protein (GST control, FIG. 3, Panel 3) were compared to the respective pre-immune sera. Negative controls never showed a shift of the histogram as compared to pre-immune sera. The control results indicated the specificity and reliability of the flow cytometric assay we set up.

We then analyzed all sera raised against recombinant C. trachomatis antigens for their capability to recognize surface exposed proteins on purified EBs, as determined by FACS binding assay. All the proteins that showed a K-S score higher than 8.0 have been listed as FACS positive, being the difference between the testing and the control histograms statistically significant (p<0.05). Of 137 different gene products analyzed, 28 showed to be able to induce antibodies capable of binding to the surface of purified EBs. Proteins that showed a positive result have been listed in Tables 1(a) and 1(b). The protein list in Table 1(a) is divided into two sections: (i) proteins that gave a positive result in the FACS assay and/or in the neutralization assay, therefore considered to be possibly surface exposed and with a neutralizing effect; (ii) proteins that showed to be able to induce antibodies directed versus surface exposed proteins of the EBs but did not show a detectable neutralizing effect. A comparative analysis of the proteins that resulted to be surface exposed in the C. trachomatis genomic screening shows that 21 out of 28 FACS positive antigens have a degree of homology higher than 40% to C. pneumoniae proteins that, as published in our previous work (Montigiani, et al., 2002), are likely surface exposed.

Analysis of the antisera to the recombinant antigens by Western blotting: The panel of sera was also screened by Western blot analysis on whole protein extracts of purified chlamydial EBs, in order to visualize their capability to recognize a band of the expected molecular weight. The results of this analysis are reported in Tables 1(a) and 1(b), while the Western blot profiles are shown in FIG. 1. In total, 22 out of the 30 sera described in Table 1(a) resulted to be “consistent”, that is they appeared to recognize a band of the expected molecular weight on EBs protein extracts. Four sera, (anti-CT547, anti-CT266, anti-CT444, anti-CT823) were classified as “partially consistent”, due to the presence of a band at the expected molecular mass plus few different bands of weaker intensity. Finally, four sera gave a negative Western blot pattern (anti-CT467, anti-CT456, anti-CT812, anti-CT823). Three out of the four Western blot negative sera (anti-CT456, anti-CT812, anti-CT823) gave a positive result in the FACS binding assay, even if with not very high K-S scores (K-S<15). It is worth noting that two of the Western blot negative sera were raised against antigens (CT812, CT823) belonging to the Pmp family (PmpD and PmpG), a Chlamydia specific family of complex proteins many of which have already been localized on the chlamydial cell surface at least in C. pneumoniae (See, e.g., Knudsen et al., (1999) Infect Immun 67, 375-83; Christiansen et al., (1999) Am Heart J 138, S491-5; Mygind, et al., (2000) FEMS Microbiol Lett 186, 163-9; and Vandahl, et al., (2002) BMC Microbiol 2, 36). The Western blot negative serum obtained by immunization with CT467 (AtoS) was scored as negative also in the FACS assay, but surprisingly it showed a high neutralizing titer (FIG. 2).

Evaluation of the antisera for in vitro neutralizing properties: An in vitro neutralization assay on purified C. trachomatis EBs allowed us to identify neutralizing antigens. Infectious EBs were pre-incubated with the mouse antisera obtained with C. trachomatis recombinant antigens and then tested for their capability to infect a monolayer of epithelial cells. By using this assay, as summarized in Table 1(a) (section 1) 9 sera have proved to be effectively neutralizing at a dilution higher than 1:30. These 9 sera were obtained by immunizing mice with recombinant proteins encoded by the following C. trachomatis genes: pepA (CT045), encoding a leucyl aminopeptidase; artJ (CT381), encoding a putative extracellular solute (possibly Arginine) binding protein of an aminoacid transport system; dnaK (CT396), encoding a well described chaperonin of the hsp70 family; two “hypothetical” genes CT398 and CT547; eno (CT587), encoding a protein homologous to bacterial enolases, glycolytic enzymes that can be found also on bacterial surfaces; ompA (CT681), encoding the major outer membrane protein; CT242 (OmpH-like), encoding a protein homologue to of the OmpH family of bacterial proteins, some members of which have been reported to be chaperones involved in outer membrane biosynthesis; atoS (CT467), encoding a putative sensor member of a transport system. As shown in FIG. 2, and summarized in Table 1(a), three of the recombinant antigens (ArtJ (CT381), CT398 and AtoS (CT467)) were able to induce antibodies with high neutralizing activity (neutralizing serum titers above 1:300); four of them (DnaK (CT396), Enolase (CT587), OmpA (and OmpH-like (CT242)) induced sera with intermediate neutralizing titers (between 1:180 and 1:300), finally sera raised against two proteins (PepA (CT045) and CT547) had titers equal or less than 100. FIG. 3, on Panels 4 to 12, shows the FACS profiles of the 9 proteins that resulted to be neutralizing, demonstrating that 7 of them are able to induce antibodies directed versus the surface of EBs, while two of them (OmpH-like and AtoS) did not show this capability. The Western blot profiles, against whole-EBs protein extracts, of the sera raised against the FACS-positive neutralizing antigens (FIG. 3) resulted to be either fully consistent, i.e. with a single band of the expected molecular weight (CT045-PepA, CT381-ArtJ) or partially consistent, i.e. showing a major band of the expected molecular weight besides other bands (CT396-DnaK, CT398, CT547, CT587-Enolase, CT681-MOMP). However, in the case of CT396 (DnaK) and CT681 (MOMP), it should be noted that previous work using 2D electrophoretic mapping and either immunoblotting with a specific monoclonal (Bini, et al., (1996) Electrophoresis 17, 185-90) or spot identification by mass spectrometry (Shaw, et al., (2002) Proteomics 2, 164-86) shows that these proteins do appear in EB extracts as multiple electrophoretic species of different Mw, probably due to processing and/or post-translational modifications. Of the 3 remaining ‘partially consistent’ profiles, those obtained with the antisera to recombinant CT398 and CT547-Enolase show that the antibodies recognize predominantly a band of the expected size, whereas in the case of the hypothetical CT547 there is in fact a doubt about the specificity of the antiserum. The two FACS negative and neutralizing antigens showed a different behavior. While the Western blot profile of CT242 (OmpH-like) is fully consistent showing a single band of the expected molecular weight (FIG. 3, Panel 8), the blot of CT467 (AtoS) resulted to be completely negative (FIG. 3, Panel 9).

In the case of the anti-OmpH (CT242) serum, the apparent contradiction between FACS and Western blot profiles could be explained assuming a different sensitivity between the two assays. However, the AtoS (CT467) results remain contradictory. Considering that the above findings could be partially explained by the fact that for safety reasons the FACS analyses were performed on heath-inactivated preparations of EB and that the inactivation procedure could have totally (anti-AtoS) or partially (anti-OmpH) destroyed conformational epitopes essential for antibody binding, we also tested these antisera in a dot-blot assay (REF) using infectious EBs spotted on a nitrocellulose membrane, as described by Kawa and Stephens (Kawa and Stephens, 2002). However, the dot-blot assay results only confirmed the results obtained with the FACS assay.

Further discussion and analysis of the results of the Western Blot, FACS and In Vitro Neutralization assays and analysis as shown in Tables 1(a) and 1(b) follows below.

Tables 1(a) and 1(b) present the results of FACS and the ‘in vitro neutralization’ assays obtained from sera raised against a set of C. trachomatis recombinant fusion proteins, of which, so far, 9 “neutralizing” antigens were identified. With the exception of MOMP, none of these antigens has been previously reported as neutralizing. Previous literature also describes PorB (CT713) as a second neutralizing protein (See Kawa, D. E. and Stephens, R. S. (2002)). Antigenic topology of chlamydial PorB protein and identification of targets for immune neutralization of infectivity. (J Immunol 168, 5184-91). However, as shown in Table 1(a), the serum against our recombinant form of PorB failed to neutralize Chlamydia infection in vitro. This discrepancy may be explained considering that our recombinant antigen was water-insoluble and therefore it might have lost the correct conformation required to induce neutralizing antibodies. The possibility of a similar situation should be kept in mind also in the interpretation of data relative to the other ‘insoluble’ antigens. It is interesting to note that, besides MOMP, other proteins m this selection, including PepA, DnaK, HtrA and PorB, have been reported as proteins which are immunogenic in the course of genital tract infection in humans.

Apart from the CT antigens for which no in-vitro neutralizing data was available (CT635, CT671 and CT859—marked as ND in Table 1(b)), none of the other CT specific proteins disclosed in Table 1(b) demonstrated in-vitro neutralizing activity. However, these in-vitro results do not mean or suggest that these CT specific antigens do not or may/could not demonstrate an in-vivo protective effect especially when used in combination with one or more other CT antigens with, for example, a complementary immunological profile (see for example, the protective effect against CT challenge which was obtained when combinations of CT antigens, such as (CT242 and CT316) and (CT467 and CT444) and (CT812 and CT082) with complementary immunological profiles are used.

### Example 2

### Western Blot, FACS and In Vitro Neutralization Assay and Analysis of CT Antigens, as Shown in Table 1(b)

Table 1(b) also provides the FACS results obtained from sera raised against a set of 17 Chlamydia trachomatis recombinant fusion proteins, these being: CT016, CT017, CT043, CT082, CT153, CT262, CT276, CT296, CT372, CT398, CT548, CT043, CT635, CT671 (all Hypothetical Proteins). CT412 (Putative Outer Membrane Protein), CT 480 (Oligopeptide Binding Protein), CT859 (Metalloprotease), CT089 (Low Calcium Response Element—LcrE), CT812 (PmpD) and CT869 (PmpE). FACS analysis was carried out on either the HIS fusion and/or the GST fusion. All of these CT recombinant fusion proteins showed a K-S score higher than 8.0 and were deemed FACS positive. With the exception of CT398, CT372 and CT548 at least none of these Hypothetical proteins has been previously reported as FACS positive. In addition, the following proteins: CT050 (Hypothetical), CT165 (Hypothetical), CT711 (Hypothetical) and CT552 (Hypothetical) also showed a K-S score higher than 8.0 and were deemed FACS positive. None of these four proteins has been previously reported as FACS positive. All of these Hypothetical CT antigens are generally regarded are CT specific antigens and do not have a C. pneumoniae counterpart.

### Example 3

### Immunization with Combinations of CT Antigens from the Second, Third and Fifth Antigen Groups

The following example illustrates immunization with various combinations of CT antigens from the second, third and fifth antigen groups within a mouse model. Specifically, in this example, immunization is shown with a combination of two antigens from the second antigen group (CT242 and CT316) and a combination of one antigen from the third antigen group and one antigen from the fifth antigen group respectively (CT812 and CT082).

The methods and mouse model used in this example are discussed further below.

Mouse Model for in-vivo screening for CT protective antigens: A Mouse Model of Chlamydia trachomatis (CT) genital infection for determining in-vivo protective effect of CT antigens (resolution of a primary Chlamydia infection) was used. The model used is described as follows: Balb/c female mice 4-6 weeks old were used. The mice were immunized intra-peritoneally (ip) with a mixture of two recombinant CT antigens in the groups as set out in Table 2 below. These CT antigens were determined to be FACS positive and/or neutralizing (see Table 1(a)). Three doses of the CT antigen mixture were given. The CT antigens in Groups 1 and 2 were HIS fusion proteins. The CT antigens used in Group 3-6 were GST fusion proteins. The mice were given hormonal treatment 5 days prior to challenge with 2.5 mg of DepoProvera (medroxyprogesterone acetate).

TABLE 2
Immunization Schedule for Example 2

| | Immunising | Immunoregulatory | |
|---|---|---|---|
| Group | Composition | agent | Route of Delivery |
| 1 | CT242 (OmpH-like) + | CFA | Intra-peritoneal (i.p,) |
| | CT316 (L7/L12) | | |
| | (20 ug of each protein) | | |
| 2 | CT242 + CT316 | AlOH (200 ug) + CpG | Intra-peritoneal (i.p,) |
| | (20 ug of each protein) | (10 ug) | |
| 3 | CT467 (AtoS) + | CFA | Intra-peritoneal (i.p,) |
| | CT444 (OmcA) | | |
| | (20 ug of each protein) | | |
| 4 | CT467 + CT444 | AlOH (200 ug) + CpG | Intra-peritoneal (i.p,) |
| | (20 ug of each protein | (10 ug) | |
| 5 | CT812 | CFA | Intra-peritoneal (i.p,) |
| | (PmpD) + CT082 | | |
| | (Hypothetical) | | |
| | (20 ug of each protein) | | |
| 6 | CT812 + CT082 | AlOH (200 ug) + CpG | Intra-peritoneal (i.p,) |
| | (20 ug of each protein) | (10 ug) | |
| 7 (Negative Control) | CFA | | Intra-peritoneal (i.p,) |
| 8 (Negative Control) | AlOH (200 ug) + CpG | | Intra-peritoneal (i.p,) |
| | (10 ug) | | |
| 9 (Positive Control) | Live Chlamyida EB | | Intra-peritoneal (i.p,) |

Test Challenges: The mice were challenged intravaginally with 10⁵ IFU of purified EBs (Serovar D), 2 weeks after the last immunization dose. A read out of vaginal swabs every 7 days up to 28 days after challenge. The following assays were also carried out on pre-challenge sera: Serological analysis: FACS, WB, Neutralization assay and ELISA. The ELISA was performed by coating plates with each recombinant antigen and testing the reaction of immune sera from single mice immunized with the combination of two CT antigens. The data is expressed as the mean value calculated for each group expressed as mean ELISA units. The Chlamydia specific antibody type (IgG, IgA etc) and isotype was checked in serum post immunization but pre-challenge. The purpose of the serum studies was to determine how the mice responded to immunization with the CT antigen combinations. The purpose of the vaginal washes was to determine how the mice responded to the bacterial challenge. Chlamydia specific antibody analyses in terms of antibody type (IgG and IgA) and antibody subtype were also carried out on the vaginal washes.

Negative Controls: The negative control used was the immunoregulatory agent alone (eg CFA or AlOH and/or CpG).

Positive “live” EB controls: The positive control used was an extract from live Chlamydia Elementary Bodies (EBs). Here the mice were infected with live Chlamydia EB at the same time that the test CT combination antigenic compositions were being administered. The “live” EB positive control animals were infected for about 1.5 months (ie 6 weeks) (because 3 doses of CT antigenic combinations were administered every 2 weeks (ie over a total of 6 weeks). The animals (mice) infected with “live” EB developed a natural immunity which resolved the infection (because Chlamydia infection in mice is a transient infection). When the mice vaccinated with the CT antigenic combinations were then challenged with “live” EB, the positive control “live” EB mice were also re-challenged (ie they were given a second dose of “live” EB). As the “live” EB positive control group developed a natural immunity, generally they cleared the second re-challenge quickly. The rate of clearance of Chlamydia infection in the test mice can then be compared with the rate of clearance of infection in the EB control mice.

The results of the immunization with combinations of CT antigens of the invention are discussed below.

Results for 3×2CT antigenic combinations+CFA: Table 2 above shows the three combinations of two different CT antigens with complementary immunological profiles which are capable of providing protection against CT challenge in a mouse model of Chlamydial genital infection. The antigen combinations were administered in combination with either CFA or AlOH and CpG. The AlOH and CpG are mixed with the antigen immediately before administration.

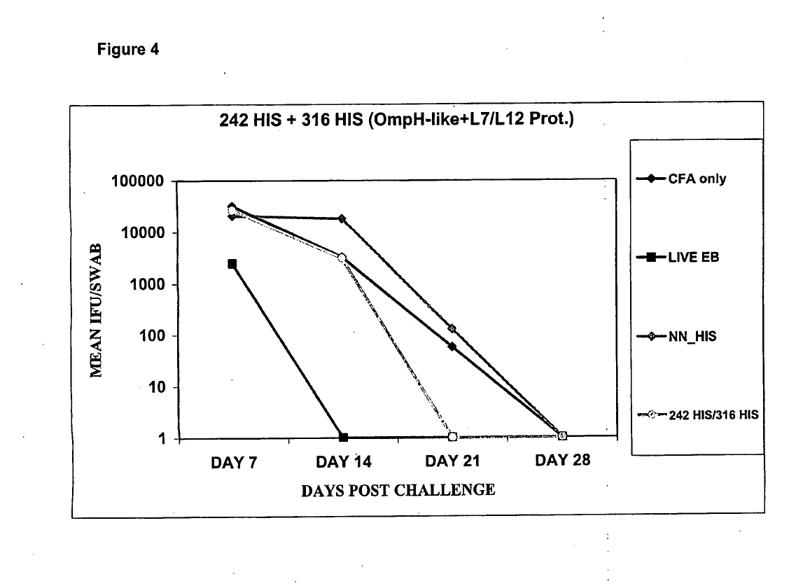

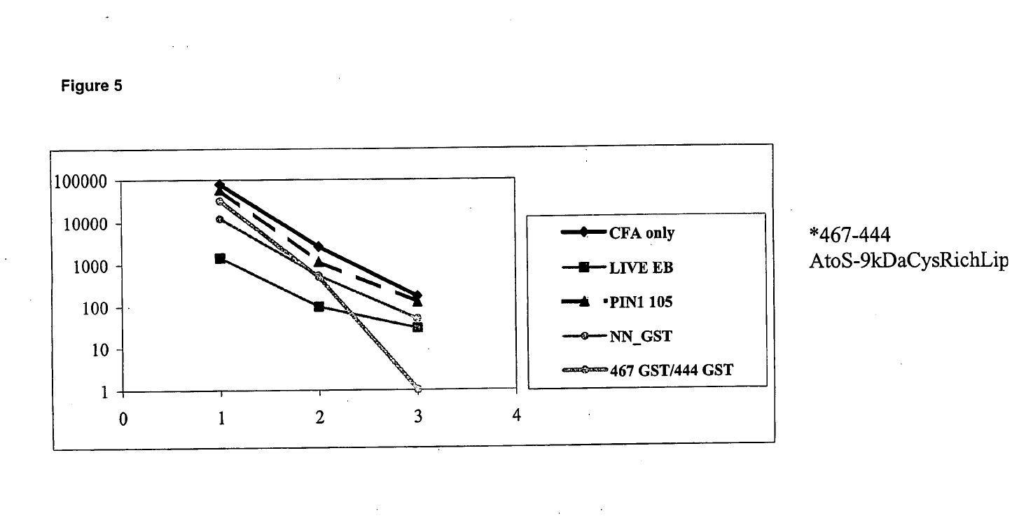

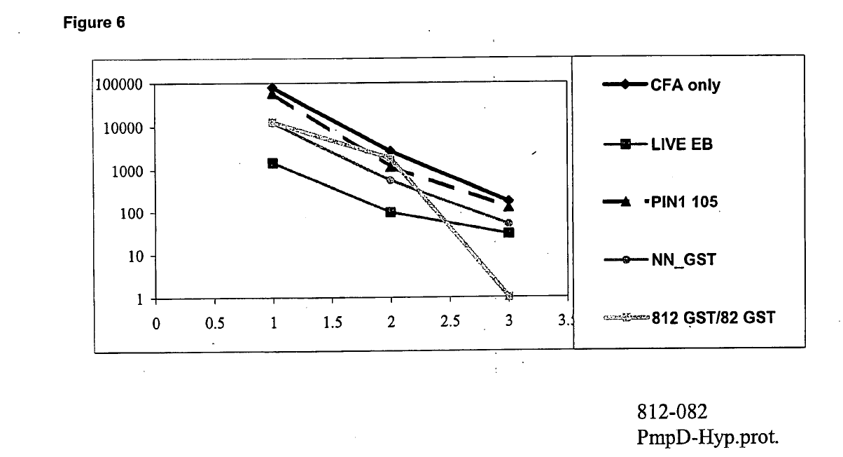

FIGS. 4-6: In the FIGS. 4-6 provided, the x axis denotes weeks post-challenge. The y axis denotes Chlamydia trachomatis units in terms of IFU/vaginal swab. The results are expressed as mean of IFU/swab recovered for each group of mice: 1=1 week or day 7. 2=2 weeks or day 14, 3=3 weeks or day 21. In each graph, both positive and negative control results are reported. A negative control=mice immunized with adjuvant alone. A positive control=mice infected with 10 (to the power of 6) Chlamydia EB IFU and rechallenged (natural protection).

The results demonstrate that a protective effect for all 3 combinations of two CT antigen was observed at 21 days post challenge.

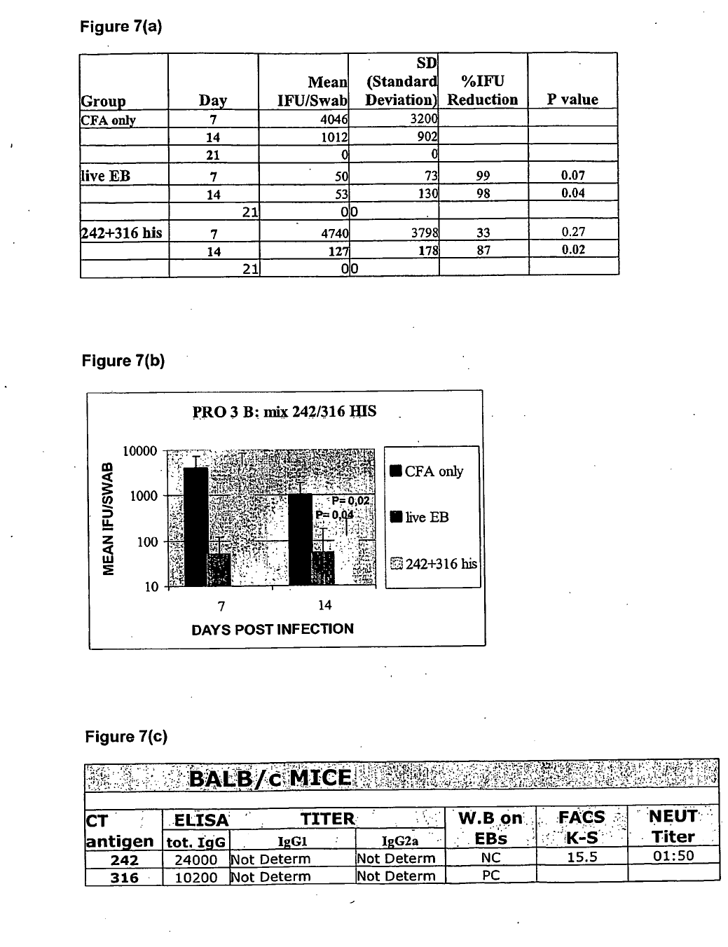

FIGS. 7(a), 7(b) and 7(c): The vaccination protocol for mice in Group 1 of Table 2 was repeated and the results obtained are set out in FIGS. 7(a)-(c). FIGS. 7(a) and 7(b) demonstrate a statistically significant protection at 14 days after CT challenge in mice immunized with a combination of CT242 and CT316 antigens and CFA adjuvant. In FIGS. 7(a) and 7(b) it is clear that at 7 days post-challenge (when Chlamydia infection is at its peak), the Chlamydia levels in the test mice vaccinated with (CT242 and CT316 and CFA) are about the same as those in the CFA controls while the EB controls show some clearance of CT infection. However, at 14 days post-challenge, the vaccinated mice have cleared the Chlamydia infection to a significant level as have the live EB control mice. It is worth noting that a stastically significant level of protection at 14 days post challenge is more meaningful than one observed at 21 days post-challenge when a much reduced level of Chlamydia bacteria is recovered from the vaginal swabs.

FIG. 7(c) indicates that the serum dilution at which a 50% reduction in infection was observed was 1:50 indicating the presence of a low in-vitro neutralizing activity for the CT214 and CT316 combination. This result indicates that a low in-vitro neutralization titre is not indicative or predictive of an in-vivo protective effect.

FIGS. 4-6 and FIG. 7(a)-(c) demonstrate that three combinations of two different CT antigen with complementary immunological profiles are capable of providing protection against CT challenge in a mouse model of Chlamydial genital infection when administered in combination with an immunoregulatory agent.

### Example 4

### Immunizations with Combinations of the First Antigen Group

The following example illustrates immunization with various combinations of CT antigens from the first antigen group within a mouse model. Specifically, in this example, immunization is shown with a combination of five antigens from the first antigen group (CT045, CT381, CT396, CT398 and CT089).

The five antigens of the first antigen group ((OmpH-like protein, ArtJ, DnaK, CT398 and HrtA) or other combinations of CT antigens as already described) were prepared as described above. The antigens are expressed and purified. Compositions of antigen combinations are then prepared comprising five antigens per composition (and containing 15 μg of each antigen per composition).

CD1 mice are divided into seven groups (5-6 mice per group for groups 1 through 6; 3 to 4 mice for groups 5, 6, 7, 8 and 9), and immunized as follows:

TABLE 3
Immunization Schedule for Example 4

| | | Route of |
|---|---|---|
| Group | Immunizing Composition | Delivery |
| 1 | Mixture of 5 antigens (15 μg/each) + CFA | Intra-peritoneal or |
| | | intra-nasal |
| 2 | Mixture of 5 antigens (15 μg/each) + AlOH | Intra-peritoneal or |
| | (200 μg) | intra-nasal |
| 3 | Mixture of 5 antigens (15 μg/each) + CpG | Intra-peritoneal or |
| | (10 ug) | intra-nasal |
| 4 | Mixture of 5 antigens (15 μg/each) + AlOH | Intra-peritoneal or |
| | (200 μg) + CpG (10 μg) | intra-nasal |
| 5 | Complete Freunds Adjuvant (CFA) | Intra-peritoneal or |
| | | intra-nasal |
| 6 | Mixture of 5 antigens (5 μg/each) + LTK63 | Intra-peritoneal or |
| | (5 μg) | Intranasal |
| 7 | AlOH (200 μg) + CpG (10 μg) | Intra-peritoneal or |
| | | intra-nasal |
| 8 | CpG (10 μg) | Intra-peritoneal or |
| | | intra-nasal |
| 9 | LTK63 (5 μg) | Intra-peritoneal or |
| | | intra-nasal |

Mice are immunized at two week intervals. Two weeks after the last immunization, all mice are challenged by intravaginal infection with Chlamydia trachomatis serovar D. When mucosal immunization (eg intra-nasal(in)) is used, the animal model is also challenged mucosally to test the protective effect of the mucosal immunogen.

### Example 5

### Immunization with Combinations of the First Antigen Group

The following example illustrates immunization with various combinations of CT antigens from the first antigen group within a mouse model. Specifically, in this example, immunization is shown with a combination of five antigens from the first antigen group (CT045, CT381, CT396, CT398 and CT089).

Mouse Model for in-vivo screening for CT protective antigens: A Mouse Model of Chlamydia trachomatis genital infection for determining in-vivo protective effect of CT antigens (resolution of a primary Chlamydia infection) was used. The model used is described as follows: Balb/c female mice 4-6 weeks old were used. The mice were immunized intra-peritoneally (ip) with a mixture of five recombinant CT antigens as set out in Table 4 below. These CT antigens were determined to be FACS positive and/or neutralizing (see Table 1(a)). Three doses of the CT five antigen mixture were given at a concentration of 15 ug per dose. The CT antigens listed in Groups 1-3 of Table 4 were HIS fusion proteins. The mice were given hormonal treatment 5 days prior to challenge with 2.5 mg of DepoProvera (medroxyprogesterone acetate).

TABLE 4
Immunization Schedule for Example 5

| | | ImmunoRegulatory | |
|---|---|---|---|
| Group | Immunising Composition | Agent | Route of Delivery |
| 1 (Test Group) | CT045 + CT089 + CT396 + | CFA | Intra-peritoneal (i.p.) |
| | CT398 + CT381 | | |
| | (15 ug/each CT antigen) | | |
| 2 (Test Group) | CT045 + CT089 + CT396 + | AlOH (200 ug) and CpG | Intra-peritoneal (i.p.) |
| | CT398 + CT381 | (10 ug) | |
| | (15 ug/each CT antigen) | | |
| 3 (Test Group) | CT045 + CT089 + CT396 + | AlOH (200 ug) alone | Intra-peritoneal (i.p.) |
| | CT398 + CT381 | | |
| | (15 ug/each CT antigen) | | |
| 4 (Test Group) | CT045 + CT089 + CT396 + | CpG (10 ug) alone | Intra-peritoneal (i.p.) |
| | CT398 + CT381 | | |
| | (15 ug/each CT antigen) | | |
| 5 | Complete Freunds | | Intra-peritoneal (i.p.) |
| (Negative control) | Adjuvant (CFA) alone | | |
| 6 | AlOH (200 μg) + CpG | | Intra-peritoneal (i.p.) |
| (Negative Control) | (10 μg) | | |
| 7 | Live Elementary Body | | Intra-peritoneal (i.p.) |
| (Positive Control) | (EB) from Chlamydia | | |
| (Protection control) | (twice - pre-challenge + | | |
| | challenge) | | |
| 8 (Infection control) | Live Elementary Body | | Intra-peritoneal (i.p.) |
| | (EB) from Chlamydia | | |
| | (once - challenge only) | | |

Test Challenges: The mice were challenged intravaginally with 10⁵ IFU of purified EBs (Serovar D), 2 weeks after the last immunization dose. A read out of vaginal swabs every 7 days up to 28 days after challenge. The following assays were also carried out on pre-challenge sera: Serological analysis: FACS, WB, Neutralization assay and ELISA. The ELISA were performed by coating plates with each recombinant antigen and testing the reaction of pre-challenge immune sera from single mice immunized with the combination of five CT antigens. The data is expressed as the mean value calculated for each group expressed as mean ELISA units. The Chlamydia specific antibody type (IgG, IgA etc) and isotype was checked in serum post immunization but pre-challenge. The purpose of the serum studies was to determine how the mice responded to immunization with the CT antigen combinations. The purpose of the vaginal washes was to determine how the mice responded to the Chlamydia bacterial challenge. Chlamydia specific antibody analyses in terms of antibody type (IgG and IgA) and antibody subtype were also carried out on the vaginal washes.

Negative Controls: The negative control used was the immunoregulatory agent alone (eg CFA or AlOH and/or CpG).

Positive “live” EB controls: The positive control used was an extract from live Chlamydia Elementary Bodies (EBs). Here the mice were infected with live Chlamydia EB at the same time that the test CT combination antigens are being administered. The “live” EB positive control animals were infected for about 1.5 months (ie 6 weeks) (because 3 doses of CT antigenic combinations were administered every 2 weeks (ie over a total of 6 weeks). The animals (mice) infected with “live” EB developed a natural immunity and resolved the infection (because Chlamydia infection in mice is a transient infection). When the mice were vaccinated with the CT antigenic combinations were then challenged with “live” EB, the positive control “live” EB mice were also re-challenged (ie they were given a second dose of “live” EB). As the “live” EB positive control group developed a natural immunity, they cleared the second re-challenge quickly.

Infection Control: In this group, the mice were only challenged with “live” EB at the same time that the “Positive Live EB controls were re-challenged and the test CT group was challenged. The purpose of this control group was to check for a possible protective effect from the negative control group (ie the group immunized with immunoregulatory agent alone)

The results for the immunizations of this example are detailed below.

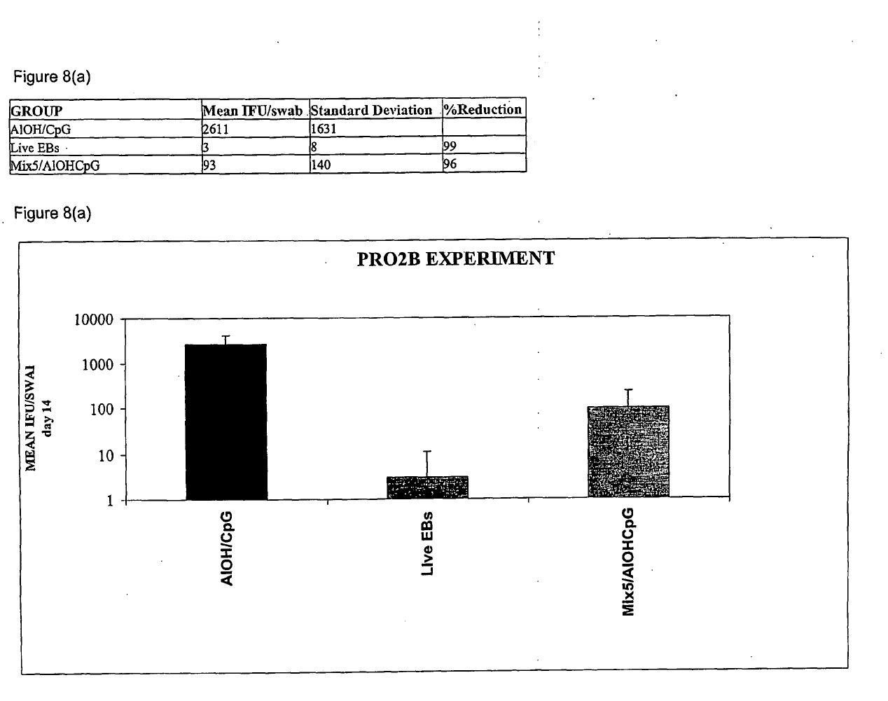

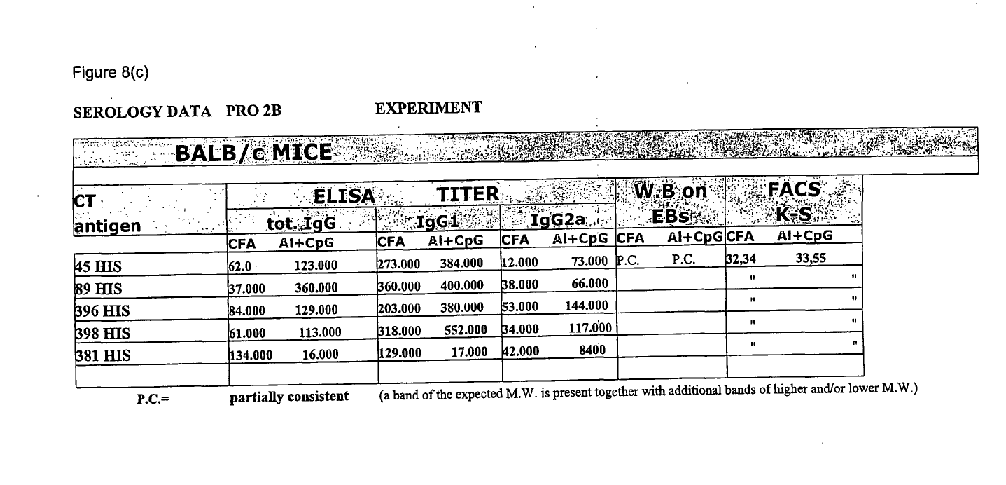

Results for 1×5 combos+CFA/AlOH+CpG: FIGS. 8(a)-8(d) show the results obtained after administration of a combination of five different CT antigens (CT045, CT089, CT396, CT398 and CT381) with complementary immunological profiles which demonstrate that this five antigen mix is capable of providing protection against CT challenge in a mouse model of Chlamydial genital infection when used in combination with an immunoregulatory agent, such as AlOH and CpG.

FIGS. 8(a), 8(b) and 8(c): In more detail: FIG. 8(b) provided, the x axis denotes results for day 14 post-challenge. The y axis denotes Chlamydia trachomatis challenge units in terms of IFU/swab at day 14. The results are expressed as mean of IFU/swab recovered for each group of mice. Both positive and negative control results are reported. A negative control=mice immunized with adjuvant alone. A positive control=mice infected with 10 (to the power of 6) Chlamydia EB IFU and rechallenged (natural protection). The results demonstrate that a protective effect for a combination of five CT antigens (CT045, CT089, CT396, CT398 and CT381) when used in combination with AlOH and CpG was observed at 14 days post challenge.

FIG. 8(c) demonstrates that Chlamydia antigen specific IgG1 and IgG2 antibody isotypes could be measured in mice serum obtained post-immunisation but pre-challenge. These Chlamydia antigen specific IgG isotype profiles are indicative of a Th2 and a Th1 protective immune response respectively. A higher level of IgG1 to IgG2 (that is, a predominance of IgG1 to IgG2) was obtained both for CFA and AlOH and CpG immunoregulatory agents with the highest IgG1 levels being obtained after administration of the 5 CT antigen mix in combination with AlOH and CpG. In addition, there was a greater fold increase in IgG2a levels for the AlOH+CpG group relative to the CFA group. Thus, the results demonstrate that an enhanced Th1 and Th2 response was observed for mice vaccinated with the 5CT antigen group and AlOH and CpG as immunoregulatory agents compared with mice vaccinated with 5CT antigen group and CFA as the immunoregulatory agent.

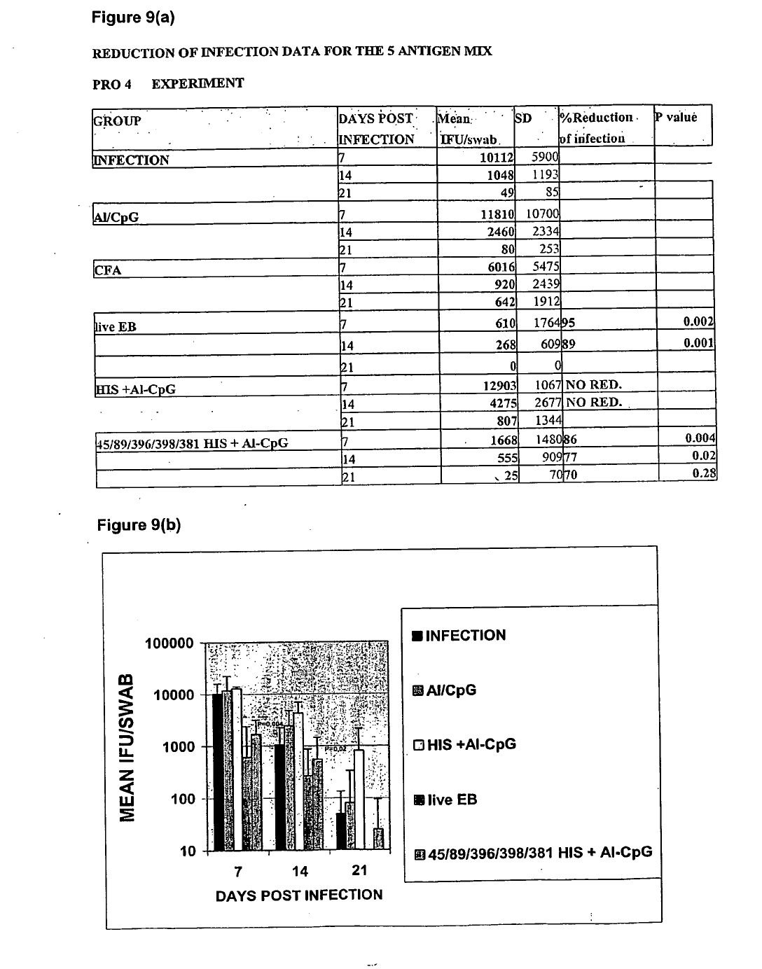

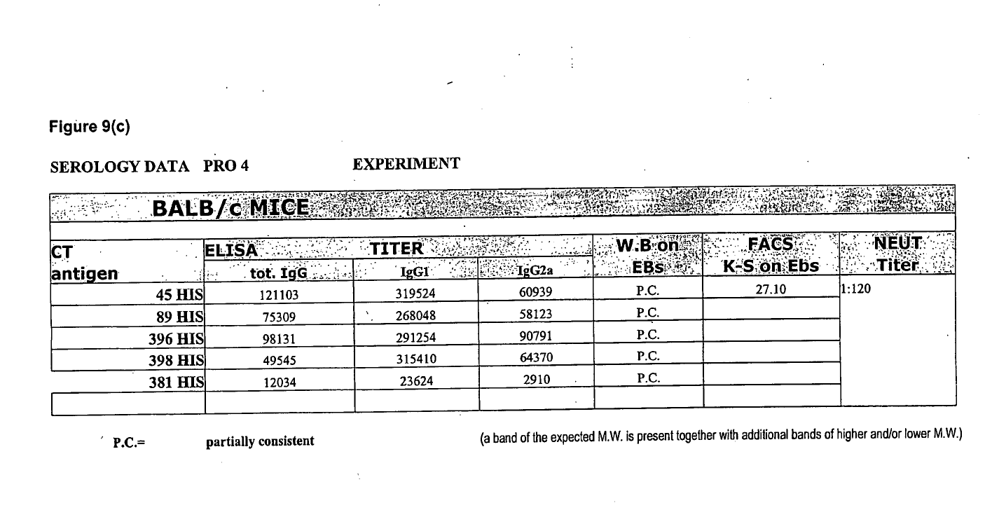

FIGS. 9(a), 9(b) and 9(c): The vaccination protocol for mice in Group 1 of Table 4 was repeated and the results obtained are set out in FIGS. 9(a)-(c). However, this time, only AlOH and CpG adjuvant was used.

FIGS. 9(a) and 9(b) demonstrate a statistically significant protection at both 7 days and 14 days after CT challenge in mice immunized with a combination of the five CT antigens (CT045, CT089, CT396, CT398 and CT381) and AlOH and CpG adjuvant. In FIG. 9(b) it is clear that at 7 days and 14 days post challenge, the vaccinated mice have cleared the Chlamydial infection to a level only slightly higher than the “live” EB positive control mice indicating that mice vaccinated with a combination of five CT antigens (CT045, CT089, CT396, CT398 and CT381) and AlOH and CpG adjuvant have almost as good a level of protective immunity as the “natural” immunity developed by the “live” EB control mice. FIG. 9(b) also demonstrates that there was a quicker and statistically significant clearance of Chlamydia infection at 7 days and 14 days post challenge. A stastically significant protective effect at 7 days post challenge is very significant finding because a Chlamydial bacterial infection in mice will peak at around 7 days post-challenge. Indeed, this is demonstrated by the EB control group which does not demonstrate a complete clearance of CT bacteria at 7 days post-challenge. A statistically significant clearance at 7 and 14 days post challenge is also far more meaningful than one observed at 21 days post challenge when the number of bacteria recovered from the vaginal swabs is relatively low.

FIG. 9(c) demonstrates that Chlamydia antigen specific IgG2a and IgG1 antibody isotypes could be measured in mice serum obtained post-immunisation but pre-challenge. These Chlamydia antigen specific IgG isotype profiles are indicative of a Th1 and a Th2 protective immune response respectively. FIG. 9(c) also indicates that the serum dilution at which a 50% reduction of Chlamydial infectivity was obtained was 1:120.

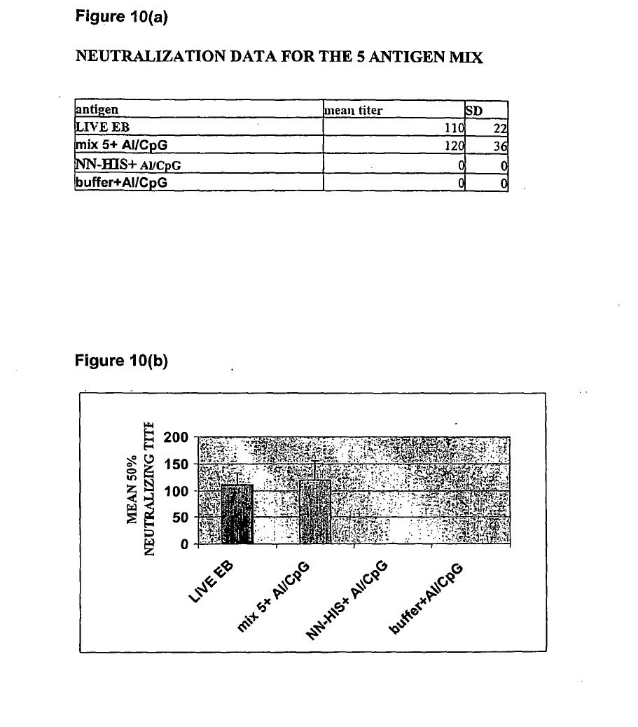

Neutralisation Data for the 5 Antigen Mix: FIGS. 10(a) and 10(b) indicate that neutralizing antibody levels obtained for the 5 CT mixture when combined with AlOH and CpG were approximately the same as those obtained for the “live” EB positive control groups whereas no neutralizing titre was detected for the negative control groups. In this regard, the serum dilutions at which a 50% reduction of Chlamydial infectivity was obtained were 1:120 and 1:110 respectively.

The results of the immunizations of this example are further discussed below.

FIGS. 8-10 demonstrate that combinations of five different CT antigens with complementary immunological profiles when used in combination with an immunoregulatory agent are capable of providing protection against CT challenge in a mouse model of Chlamydial genital infection. Without wishing to be bound by theory, it appears that the combination of AlOH and CpG elicits an enhanced IgG1 and IgG2a immune response which is indicative of an enhanced Th2 and Th1 immune response respectively.

### Overall Discussion

According to a genomic strategy aiming at the identification of new vaccine candidates, which gave promising results for other bacterial pathogens, we expressed in E. coli, as recombinant fusion proteins, 158 ORFs selected in silico from the C. trachomatis genome, and likely to encode peripherally located proteins. Polyclonal antibodies to these proteins were raised in mice and assessed, in parallel screenings, (i), for their capacity to bind purified Chlamydiae in a flow cytometry assay (identifying FACS-positive sera and corresponding antigens), and, (ii), for their capacity to induce a >50% inhibition of Chlamydial infectivity for in vitro cell cultures (neutralizing sera and antigens). The specificity of the antisera, which were partially purified by adsorption on E. coli protein extracts, was assessed by Western Blot analysis of the sera diluted 1:400 (the same dilution found optimal for the FACS assay screening) which were tested against protein extracts of gradient-purified elementary bodies of C. trachomatis. The Western Blot results showed that the majority of the 30 FACS positive and/or neutralizing antisera recognized either a single protein band of expected molecular size, or that a band consistent with the expected chlamydial antigen was anyway predominant in the WB profile, with only minor bands of different size. In fact only for 5 antigens a doubt remained as to the true specificity of the antiserum, namely in the case of the CT547 protein, for which the expected band was present but not predominant, and the 4 cases for which the WB obtained was completely blank (CT456, CT476-AtoS, and the two fusion proteins for pmpD (CT812) and pmpE (CT869).

The parallel screenings identified FACS-positive sera and corresponding antigens, and, so far, 9 ‘neutralizing’ antisera and antigens (Table1(a)). Seven of these (the recombinant forms of PepA (CT045), ArtJ (CT381), DnaK (CT396), Enolase (CT587); the 2 hypothetical products of CT398 and CT547, and the well studied product of ompA better known as the Major Outer Membrane Protein, MOMP (CT681), of C. trachomatis) were both FACS-positive and neutralizing in vitro: the neutralization data therefore seem to confirm that the binding observed in the FACS assay occurred to intact infectious EBs. On the contrary, the two recombinant antigens obtained for the OmpH-like (CT242) and AtoS (CT467) proteins elicited antibodies with in vitro neutralizing properties, but surprisingly failed to show any measurable binding in the FACS assay (FIGS. 2 and 3). The results obtained for CT242 and CT467 are surprising and unexpected as these antigens appear not to be surface-exposed and yet both have high in-vitro neutralizing titres.

AtoS (CT467): AtoS is a particular case in that the antiserum failed to detect any protein species by Western Blot analysis, and gave negative FACS assay results (with a K-S score below cut-off threshold). Nevertheless this antiserum yielded one of the best neutralization titres, second only to that elicited by the CT398 ‘hypothetical’ protein. Interestingly, in the previous similar screening on Chlamydia pneumoniae (Cpn) antigens (Montigiani et al (2002) Infect Immun 70: 368-379), the antiserum to the homolog Cpn-AtoS proved again to be WB negative, but in this case FACS positive (KS=14.61) and capable of neutralizing (average titre=270) Cpn in vitro infection of the same cell line used in the present study. The apparent inconsistency of these results may be explained by considering that an antigen present in very small amounts in the EB sample could bind too little antibody to be detected in the FACS binding assay, however it could become detectable by the in vitro neutralization assay owing to the possibility of using higher concentration of antibodies and to the amplification provided by the chlamydial replication in this type of assay. The hypothesis that AtoS is somehow lost in purified EBs, e.g. due to a particular instability, is in agreement with the fact that the AtoS protein, shown to be the sensor moiety of a 2-component system composed by AtoS and AtoC was never observed so far by mass spectrometry analysis of 2DE proteomic map nor in any of 3 CT serotypes whereas the expression of the presumably equally abundant AtoC subunit was detected in the 2DE map of serotype-A CT by MALDI-TOF analysis.

CT08 (Hypothetical Protein): CT082 (Hypothetical Protein) is part of an operon annotated as a late transcription unit, and the expression of this ORF has been detected in the EB proteome. It is interesting that our data now indicate the likely exposure of the CT082 protein on the EB surface, supported by a relatively high K-S score (25.62) in the FACS assay. This localization together with its late expression in the replicative cycle suggests an important role of CT082 for some of the multiple EB functions. Surprisingly, we could not detect a sufficient infectivity neutralization mediated by our anti CT082 antiserum. However, as pointed out above, a negative results in a screening study is not to be taken as definitive because many factors (type of recombinant expression, quality of antibody response, the necessarily artificial conditions of the in vitro neutralization assay) may influence the outcome and affect the sensitivity of these assays.

CT398 (Hypothetical Protein): The CT398 antiserum yielded the best neutralization titre in this study. The biological function of this hypothetical protein is unknown. However its presence in the EB proteome has been confirmed by mass spectrometry analysis. Our data now indicate its surface localization and neutralizing properties, and in silico analysis, although an N-terminal signal peptide is not detected by algorithms like PSORT, indicates the presence of a predicted coiled-coil structure between amino-acid residues 11 and 170 which is often present in bacterial surface proteins. Homology searches indicate some homology to a human muscle protein (MYST_HUMAN) and a structural similarly hit with gi|230767|pdb|2TMA|A Chain A, Tropomyosin.

The negative results obtained in these studies are to be considered only negative in relation to the specific procedures and conditions adopted in the screening tests. That is, a negative result may simply be a function of the assay sensitivity. A typical example of such situation is represented by the recombinant porB protein (a conserved dicarboxylate-specific porin which may feed the Chlamydial TCA cycle) which in our hands proved to be surface exposed, in agreement with published data but unable to induce neutralizing antibodies. However, as shown by other workers in the field, porB is in fact also a neutralizing antigen. The discrepancy can be explained considering that the recombinant porB used in these studies. In order to display its neutralizing activity, the initially insoluble recombinant porB had to be refolded by extraction with 1% octlyglucoside and a dialysis step against PBS. Therefore, the neutralizing activity of porB clearly depends on its folding and in our screening work we may have obtained a recombinant porB with a folding which allowed the detection of surface exposure in the FACS assay but lost the neutralizing epitope(s). A similar situation could have been envisaged, from literature data, for the other known porin of Chlamydia, that is for the ompA gene product MOMP (CT681), the best studied vaccine candidate so far, which was also described as possessing folding dependent neutralization properties. Accordingly, one could have expected that in absence of specific refolding steps, our screening results could have failed to detect recombinant MOMP as neutralizing. This however was not the case, and in fact the presence of MOMP within the short list of neutralizing antigens acquires in a way the value of an internal positive control.

The project described herein took advantage from previous work by selecting as a first option a number of C. trachomatis genes considered orthologous (up to 40% identity in the encoded polypeptide) to ‘FACS-positive’ genes of C. pneumoniae, i.e. to genes which when expressed as GST or (6)His fusion proteins elicited antibodies binding to purified C. pneumoniae cells. In Table 1(a) the names of CT proteins which had a corresponding positive screening results in C. pneumoniae are shaded, and it can be noted that 70% of the CT FACS-positive antigens we report have a Cpn ortholog previously described as FACS-positive. For general comments on the types of proteins so detected as potential constituents of the chlamydial EB surface, and degree of expected agreement of these experimental finding with the current in silico annotations, we therefore refer the reader to the discussion of the previous results (Montigiani et al (2002) ibid). As far as the neutralization assay is concerned, the published Cpn work did not included this type of assay, however subsequent work from our laboratory identified in the FACS-positive set, at least 10 Cpn neutralizing antigens (Finco et al, submitted). It is noteworthy that the AtoS, ArtJ, Enolase and OmpH-like antigens (4 of the 9 neutralizing antigens identified in this study) when expressed as Cpn specific allelic variants have neutralizing properties for Cpn in vitro infectivity as well. In contrast with the precedent C. pneumoniae study, when the majority of the Cpn Pmp's yielded soluble and ‘FACS-positive’ fusion proteins, in the present study we obtained only 4 FACS-positive Pmp fusions proteins out of 9 Pmps identified in the CT genome.

### Overall Summary

The present invention demonstrates that combinations of CT antigens are protective against Chlamydia challenge. These CT antigenic combinations are capable of inducing both a antibody response (in terms of neutralising antibody) and a cellular mediated immune response (at least in terms of a Th1 cellular profiles) which can quickly respond upon exposure to Chlamydia.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be covered by the present invention.

1. An immunogenic composition comprising a combination of Chlamydia trachomatis antigens, said combination consisting of two, three, four or all five Chlamydia trachomatis antigens of a first antigen group, said first antigen group consisting of PepA, LcrE, ArtJ, DnaK and CT398.

2. The composition of claim 1, wherein said combination consists of PepA, LcrE, ArtJ, DnaK and CT398.

3. The composition of claim 1, wherein said combination includes LcrE.

4. The composition of claim 1, said composition further comprising one or more immunoregulatory agents.

5. The composition of claim 4, wherein said one or more immunoregulatory agents include an adjuvant.

6. The composition of claim 5, wherein said adjuvant is selected from the group consisting of a TH1 adjuvant and a TH2 adjuvant.

7. The composition of claim 5, wherein the adjuvant is selected from the group consisting of aluminum salts and oligonucleotides comprising CpG motifs.

8. An immunogenic composition comprising a combination of Chlamydia trachomatis antigens, said combination consisting of two, three, four, five, six, seven, eight, nine, ten, eleven, twelve, or thirteen Chlamydia trachomatis antigens of a second antigen group, said second antigen group consisting PepA, LcrE, ArtJ, DnaK, CT398, OmpH-like, L7/L12, OmcA, AtoS, CT547, Enolase, HtrA and MurG.

9. The immunogenic composition of claim 8, wherein said combination includes one or more of the group consisting of PepA, LcrE, ArtJ, DnaK, OmpH-like and CT398.

10. The immunogenic composition of claim 8, wherein said combination includes LcrE.

11. The immunogenic composition of claim 8, wherein said combination includes OmpH-like protein.

12. The composition of claim 8, said composition further comprising one or more immunoregulatory agents.

13. The composition of claim 12, wherein said one or more immunoregulatory agents include an adjuvant.

14. The composition of claim 13, wherein said adjuvant is selected from the group consisting of a TH1 adjuvant and a TH2 adjuvant.

15. The composition of claim 13, wherein the adjuvant is selected from the group consisting of aluminum salts and oligonucleotides comprising CpG motifs.

16. A vaccine comprising the immunogenic composition of any one of the previous claims.

17. Use of the immunogenic composition according to any one of claim 1-15 or a vaccine according to claim 16 in the preparation of a medicament for the prevention or treatment of a Chlamydia trachomatis infection.

18. A method of neutralizing a Chlamydia trachomatis infection in a mammal comprising the step of administering to the mammal an effective amount of the composition of any one of claims 1 to 15 or the vaccine according to claim 16 or antibodies which recognize an immunogenic composition as defined in any one of claims 1 to 15.

19. A method of raising an immune response in a mammal against a Chlamydia trachomatis infection comprising administering to the mammal an effective amount of the composition of any one of claims 1 to 15 or the vaccine according to claim 16 or antibodies which recognizes an immunogenic composition as defined in any one of claims 1 to 15.

20. The method of claim 19, wherein said composition elicits an enhanced TH1 and TH2 immune response.

21. A method of raising Chlamydia trachomatis specific antibodies comprising administering to the mammal an effective amount of the composition of any one of claims 1 to 15 or the vaccine according to claim 16 or an antibody which recognizes a protein as defined in any one of claims 1 to 15.

22. An immunogenic composition comprising a combination of Chlamydia trachomatis antigens, said combination consisting of two, three, four or all five Chlamydia trachomatis antigens of a first antigen group, said first antigen group consisting of PepA, LcrE, ArtJ, DnaK and CT398, wherein said composition further comprising one or more immunoregulatory agents.

23. An immunogenic composition comprising an oligonucleotide containing a CpG motif, a mineral salt, and an antigen associated with a sexually transmissible disease.

24. The composition of claim 23, wherein said mineral salt is an aluminum salt.

25. The composition of claim 23, wherein said antigen is a Chlamydia trachomatis antigen.


SEQUENCE LISTING (61 sequences)

SEQ ID NO: 1 - protein, 499 aa, Chlamydia trachomatis
MVLLYSQASWDKRSKADALVLPFWMKNSKAQEAAVVDEDYKLVYQNALSNFSGKKGETAFLFGNDHTKEQKIVLLGLGKSEEVSGTTVLEAYAQATTVLRKAKCKTVNILLPTISQLRFSVEEFLTNLAAGVLSLNYNYPTYHKVDTSLPFLEKVTVMGIVSKVGDKIFRKEESLFEGVYLTRDLVNTNADEVTPEKLAAVAKDLAGEFASLDVKILDRKAILKEKMGLLAAVAKGAAVEPRFIVLDYQGKPKSKDRTVLIGKGVTFDSGGLDLKPGKAMITMKEDMAGAATVLGIFSALASLELPINVTGIIPATENAIGSAAYKMGDVYVGMTGLSVEIGSTDAEGRLILADAISYALKYCNPTRIIDFATLTGAMVVSLGESVAGFFANNDVLARDLAEASSETGEALWRMPLVEKYDQALHSDIADMKNIGSNRAGSITAALFLQRFLEDNPVAWAHLDIAGTAYHEKEELPYPKYATGFGVRCLIHYMEKFLSK

SEQ ID NO: 2 - protein, 421 aa, Chlamydia trachomatis
MTASGGAGGLGSTQTVDVARAQAAAATQDAQEVIGSQEASEASMLKGCEDLINPAAATRIKKKGEKFESLEARRKPTADKAEKKSESTEEKGDTPLEDRFTEDLSEVSGEDFRGLKNSFDDDSSPDEILDALTSKFSDPTIKDLALDYLIQTAPSDGKLKSTLIQAKHQLMSQNPQAIVGGRNVLLASETFASRANTSPSSLRSLYFQVTSSPSNCANLHQMLASYLPSEKTAVMEFLVNGMVADLKSEGPSIPPAKLQVYMTELSNLQALHSVNSFFDRNIGNLENSLKHEGHAPIPSLTTGNLTKTFLQLVEDKFPSSSKAQKALNELVGPDTGPQTEVLNLFFRALNGCSPRIFSGAEKKQQLASVITNTLDAINADNEDYPKPGDFPRSSFSSTPPHAPVPQSEIPTSPTSTQPPSP

SEQ ID NO: 3 - protein, 257 aa, Chlamydia trachomatis
MCIKRKKTWIAFLAVVCSFCLTGCLKEGGDSNSEKFIVGTNATYPPFEFVDKRGEVVGFDIDLAREISNKLGKTLDVREFSFDALILNLKQHRIDAVITGMSITPSRLKEILMIPYYGEEIKHLVLVFKGENKHPLPLTQYRSVAVQTGTYQEAYLQSLSEVHIRSFDSTLEVLMEVMHGKSPVAVLEPSIAQVVLKDFPALSTATIDLPEDQWVLGYGIGVASDRPALALKIEAAVQEIRKEGVLAELEQKWGLNN

SEQ ID NO: 4 - protein, 660 aa, Chlamydia trachomatis
MSEKRKSNKIIGIDLGTTNSCVSVMEGGQPKVIASSEGTRTTPSIVAFKGGETLVGIPAKRQAVTNPEKTLASTKRFIGRKFSEVESEIKTVPYKVAPNSKGDAVFDVEQKLYTPEEIGAQILMKMKETAEAYLGETVTEAVITVPAYFNDSQRASTKDAGRIAGLDVKRIIPEPTAAALAYGIDKEGDKKIAVFDLGGGTFDISILEIGDGVFEVLSTNGDTHLGGDDFDGVIINWMLDEFKKQEGIDLSKDNMALQRLKDAAEKAKIELSGVSSTEINQPFITIDANGPKHLALTLTRAQFEHLASSLIERTKQPCAQALKDAKLSASDIDDVLLVGGMSRMPAVQAVVKEIFGKEPNKGVNPDEVVAIGAAIQGGVLGGEVKDVLLLDVIPLSLGIETLGGVMTPLVERNTTIPTQKKQIFSTAADNQPAVTIVVLQGERPMAKDNKEIGRFDLTDIPPAPRGHPQIEVTFDIDANGILHVSAKDAASGREQKIRIEASSGLKEDEIQQMIRDAELHKEEDKQRKEASDVKNEADGMIFRAEKAVKDYHDKIPAELVKEIEEHIEKVRQAIKEDASTTAIKAASDELSTHMQKIGEAMQAQSASAAASSAANAQGGPNINSEDLKKHSFSTRPPAGGSASSTDNIEDADVEIVDKPE

SEQ ID NO: 5 - protein, 254 aa, Chlamydia trachomatis
MHDALQSILAIQELDIKMIRLMRVKKEHQNELAKIQALKTDIRRKVEEKEQEMEKLKDQIKGGEKRIQEISDQINKLENQQAAVKKMDEFNALTQEMTAANKERRTLEHQLSDLMDKQAGSEDLLISLKESLSSTENSSSAIEEEIRENIRKINEEGRSLLSQRTQLKETTDPELFSIYERLLNNKKDRVVVPIENRVCSGCHIALTPQHENLVRKQDHLVFCEHCSRILYWQELQSPSAEGATTKRRRRRTAV

SEQ ID NO: 6 - protein, 173 aa, Chlamydia trachomatis
MKKFLLLSLMSLSSLPTFAANSTGTIGIVNLRRCLEESALGKKESAEFEKMKNQFSNSMGKMEEELSSIYSKLQDDDYMEGLSETAAAELRKKFEDLSAEYNTAQGQYYQILNQSNLKRMQKIMEEVKKASETVRIQEGLSVLLNEDIVLSIDSSADKTDAVIKVLDDSFQNN

SEQ ID NO: 7 - protein, 130 aa, Chlamydia trachomatis
MTTESLETLVEQLSGLTVLELSQLKKLLEEKWDVTAAAPVVAVAGAAAAGDAPASAEPTEFAVILEDVPSDKKIGVLKVVREVTGLALKEAKEMTEGLPKTVKEKTSKSDAEDTVKKLQEAGAKAVAKGL

SEQ ID NO: 8 - protein, 88 aa, Chlamydia trachomatis
MKKTALLAALCSVVSLSSCCRIVDCCFEDPCAPIQCSPCESKKKDVDGGCNSCNGYVPACKPCGGDTHQDAKHGPQARGIPVDGKCRQ

SEQ ID NO: 9 - protein, 352 aa, Chlamydia trachomatis
MPKIDTCDSCVSNTELLAIRTRVTQSYNEAQTILSSIPDGIFLLSESGEILICNPQARAILGIPEDIQLVTRMFHDFFPDTFFGFSVQEALEKEVPPKTIRLTLSQELSQKEVEVFVRKNISHDFLFLLIRDRSDYRQLEQAIEKYRSISELGKIAATLAHEIRNPLTSISGFATLLKEELSSERHQRMLNVIIEGTRSLNSLVSSMLEYTKIQPLNLRSIDLQDFFSSLIPELSLTFPSCTFRRTILSPIQRSIDPDRLRCVIWNLVKNAVEASDEEIFLELHEKGFSVINTGTLPPNIQEKLFIPFFTTKPQGNGLGLAEAHKIMRLHGGDLVVSTQDNRTTFTILWTPA

SEQ ID NO: 10 - protein, 318 aa, Chlamydia trachomatis
MKVILRALCLFLVLPCGCYARVPSFEPFRGAIAPNRYTPKHSPELYFEMGDKYFQAKKFKQALLCFGMITHHFPEHALHPKAQFLVGLCYLEMGHPDLADKALTQYQELADTEYSEQLFAIKYSIAQSFANGKRKNIVPLEGFPKLLKADTDALRIFEEIVTASSDADLKASALYAKGALLFDRKEYSEAIKTLKKVSLQFPSHSLSPESFTLIAKIHCLQALQEPYNEQYLQDARMNAAALRKQHPNHPSNTEVENYIHHMCEAYASCLYSTGRFYEKKRKASSAKIYYSIALENFPDTSYVAKCNKRLERLSKQMS

SEQ ID NO: 11 - protein, 424 aa, Chlamydia trachomatis [sequence omitted]

SEQ ID NO: 12 - protein, 497 aa, Chlamydia trachomatis
MMKRLLCVLLSTSVFSSPMLGYSASKKDSKADICLAVSSGDQEVSQEDLLKEVSRGFSRVAAKATPGVVYIENFPKTGNQAIASPGNKRGFQENPFDYFNDEFFNRFFGLPSHREQQRPQQRDAVRGTGFIVSEDGYVVTNHHVVEDAGKIHVTLHDGQKYTAKIVGLDPKTDLAVIKIQAEKLPFLTFGNSDQLQIGDWAIAIGNPFGLQATVTVGVISAKGRNQLHIVDFEDFIQTDAAINPGNSGGPLLNINGQVIGVNTAIVSGSGGYIGIGFAIPSLMAKRVIDQLISDGQVTRGFLGVTLQPIDSELATCYKLEKVYGALVTDVVKGSPAEKAGLRQEDVIVAYNGKEVESLSALRNAISLMMPGTRVVLKIVREGKTIEIPVTVTQIPTEDGVSALQKMGVRVQNITPEICKKLGLAADTRGILVVAVEAGSPAASAGVAPGQLILAVNRQRVASVEELNQVLKNSKGENVLLMVSQGDVVRFIVLKSDE

SEQ ID NO: 13 - protein, 352 aa, Chlamydia trachomatis
MKKINKIVLAVGGTGGHIIPALAARETFIHEDIEVLLLGKGLAHFLGDDSEVAYCDIPSGSPFSLRVNRMFSGAKQLYKGYVAALQKIRDFTPDLAIGFGSYHSLPAMLASIRSRIPLFLHEQNIVPGKVNKLFSRFAKGVGMSFAAAGEHFHCRAEEVFLPIRKLSEQIVFPGASPVICVVGGSQGAKILNDVVPKALARIRESYSNLYVHHIVGPKGDLQAVSQVYQDAGINHTVTAFDHNMLGVLQASDLVISRSGATMLNELLWVQVPAILIPYPGAYGHQEVNAKFFTHTVGGGTMILQKYLTEESLSKQVLLALDPATSENRRKAMLSAQQKKSFKSLYQFICESL

SEQ ID NO: 14 - protein, 264 aa, Chlamydia trachomatis
MGNSGFYLYNTQNCVFADNIKVGQMTEPLKDQQIILGTTSTPVAAKMTASDGISLTVSNNPSTNASITIGLDAEKAYQLILEKLGDQILGGIADTIVDSTVQDILDKITTDPSLGLLKAFNNFPITNKIQCNGLFTPRNIETLLGGTEIGKFTVTPKSSGSMFLVSADIIASRMEGGVVLALVREGDSKPYAISYGYSSGVPNLCSLRTRIINTGLTPTTYSLRVGGLESGVVWVNALSNGNDILGITNTSNVSFLEVIPQTNA

SEQ ID NO: 15 - protein, 975 aa, Chlamydia trachomatis
MNRVIEIHAHYDQRQLSQSPNTNFLVHHPYLTLIPKFLLGALIVYAPYSFAEMELAISGHKQGKDRDTFTMISSCPEGTNYIINRKLILSDFSLLNKVSSGGAFRNLAGKISFLGKNSSASIHFKHININGFGAGVFSESSIEFTDLRKLVAFGSESTGGIFTAKEDISFKNNHHIAFRNNITKGNGGVIQLQGDMKGSVSFVDQRGAIIFTNNQAVTSSSMKHSGRGGAISGDFAGSRILFLNNQQITFEGNSAVHGGAIYNKNGLVEFLGNAGPLAFKENTTIANGGAIYTSNFKANQQTSPILFSQNHANKKGGAIYAQYVNLEQNQDTIRFEKNTAKEGGGAITSSQCSITAHNTIIFSDNAAGDLGGGAILLEGKKPSLTLIAHSGNIAFSGNTMLHITKKASLDRHNSILIKEAPYKIQLAANKNHSIHFFDPVMALSASSSPIQINAPEYETPFFSPKGMIVFSGANLLDDAREDVANRTSIFNQPVHLYNGTLSIENGAHLIVQSFKQTGGRISLSPGSSLALYTMNSFFHGNISSKEPLEINGLSFGVDISPSNLQAEIRAGNAPLRLSGSPSIHDPEGLFYENRDTAASPYQMEILLTSDKIVDISKFTTDSLVTNKQSGFQGAWHFSWQPNTINNTKQKILRASWLPTGEYVLESNRVGRAVPNSLWSTFLLLQTASHNLGDHLCNNRSLIPTSYFGVLIGGTGAEMSTHSSEEESFISRLGATGTSIIRLTPSLTLSGGGSHMFGDSFVADLPEHITSEGIVQNVGLTHVWGPLTVNSTLCAALDHNAMVRICSKKDHTYGKWDTFGMRGTLGASYTFLEYDQTMRVFSFANIEATNILQRAFTETGYNPRSFSKTKLLNIAIPIGIGYEFCLGNSSFALLGKGSIGYSRDIKRENPSTLAHLAMNDFAWTTNGCSVPTSAHTLANQLILRYKACSLYITAYTINREGKNLSNSLSCGGYVGF

SEQ ID NO: 16 - protein, 1751 aa, Chlamydia trachomatis
MKWLSATAVFAAVLPSVSGFCFPEPKELNFSRVGTSSSTTFTETVGEAGAEYIVSGNASFTKFTNIPTTDTTTPTNSNSSSSNGETASVSEDSDSTTTTPDPKGGGAFYNAHSGVLSFMTRSGTEGSLTLSEIKITGEGGAIFSQGELLFTDLTGLTIQNNLSQLSGGAIFGESTISLSGITKATFSSNSAEVPAPVKKPTEPKAQTASETSGSSSSSGNDSVSSPSSSRAEPAAANLQSHFICATATPAAQTDTETSTPSHKPGSGGAIYAKGDLTIADSQEVLFSINKATKDGGAIFAEKDVSFENITSLKVQTNGAEEKGGAIYAKGDLSIQSSKQSLFNSNYSKQGGGALYVEGDINFQDLEEIRIKYNKAGTFETKKITLPKAQASAGNADAWASSSPQSGSGATTVSNSGDSSSGSDSDTSETVPATAKGGGLYTDKNLSITNITGIIEIANNKATDVGGGAYVKGTLTCENSHRLQFLKNSSDKQGGGIYGEDNITLSNLTGKTLFQENTAKEEGGGLFIKGTDKALTMTGLDSFCLINNTSEKHGGGAFVTKEISQTYTSDVETIPGITPVHGETVITGNKSTGGNGGGVCTKRLALSNLQSISISGNSAAENGGGAHTCPDSFPTADTAEQPAAASAATSTPESAPVVSTALSTPSSSTVSSLTLLAASSQASPATSNKETQDPNADTDLLIDYVVDTTISKNTAKKGGGIYAKKAKMSRIDQLNISENSATEIGGGICCKESLELDALVSLSVTENLVGKEGGGLHAKTVNISNLKSGFSFSNNKANSSSTGVATTASAPAAAAASLQAAAAAVPSSPATPTYSGVVGGAIYGEKVTFSQCSGTCQFSGNQAIDNNPSQSSLNVQGGAIYAKTSLSIGSSDAGTSYIFSGNSVSTGKSQTTGQIAGGAIYSPTVTLNCPATFSNNTASMATPKTSSEDGSSGNSIKDTIGGAIAGTAITLSGVSRFSGNTADLGAAIGTLANANTPSATSGSQNSITEKITLENGSFIFERNQANKRGAIYSPSVSIKGNNITFNQNTSTHDGSAIYFTKDATIESLGSVLFTGNNVTATQASSATSGQNTNTANYGAAIFGDPGTTQSSQTDAILTLLASSGNITFSNNSLQNNQGDTPASKFCSIAGYVKLSLQAAKGKTISFFDCVHTSTKKIGSTQNVYETLDINKEENSNPYTGTIVFSSELHENKSYIPQNAILHNGTLVLKEKTELHVVSFEQKEGSKLIMKPGAVLSNQNIANGALVINGLTIDLSSMGTPQAGEIFSPPELRIVATTSSASGGSGVSSSIPTNPKRISAAAPSGSAATTPTMSENKVFLTGDLTLIDPNGNFYQNPMLGSDLDVPLIKLPTNTSDVQVYDLTLSGDLFPQKGYMGTWTLDSNPQTGKLQARWTFDTYRRWVYIPRDNHFYANSILGSQNSMIVVKQGLINNMLNNARFDDIAYNNFWVSGVGTFLAQQGTPLSEEFSYYSRGTSVAIDAKPRQDFILGAAFSKMVGKTKAIKKMHNYFHKGSEYSYQASVYGGKFLYFLLNKQHGWALPFLIQGVVSYGHIKHDTTTLYPSIHERNKGDWEDLGWLADLRISMDLKEPSKDSSKRITVYGELEYSSIRQKQFTEIDYDPRHFDDCAYRNLSLPVGCAVEGAIMNCNILMYNKLALAYMPSIYRNNPVCKYRVLSSNEAGQVICGVPTRTSARAEYSTQLYLGPFWTLYGNYTIDVGMYTLSQMTSCGARMIF

SEQ ID NO: 17 - protein, 1770 aa, Chlamydia trachomatis
MKFMSATAVFAAALSSVTEASSIQDQIKNTDCNVSKLGYSTSQAFTDMMLADNTEYRAADSVSFYDFSTSSRLPRKHLSSSSEASPTTEGVSSSSSGETDEKTEEELDNGGIIYAREKLTISESQDSLSNQSIELHDNSIFFGEGEVIFDHRVALKNGGAIYGEKEVVFENIKSLLVEVNIAVEKGGSVYAKERVSLENVTEATFSSNGGEQGGGGIYSEQDMLISDCNNVHFQGNAAGATAVKQCLDEEMIVLLAECVDSLSEDTLDSTPETEQTESNGNQDGSSETEDTQVSESPESTPSPDDVLGKGGGIYTEKSLTITGITGTIDFVSNIATDSGAGVFTKENLSCTNTNSLQFLKNSAGQHGGGAYVTQTMSVTNTTSESITTPPLIGEVIFSENTAKGHGGGICTNKLSLSNLKTVTLTKNSAKESGGAIFTDLASIPITDTPESSTPSSSSPASTPEVVASAKINRFFASTAKPAAPSLTEAESDQTDQTETSDTNSDIDVSIENILNVAINQNTSAKKGGAIYGKKAKLSRINNLELSGNSSQDVGGGLCLTESVEFDAIGSLLSHYNSAAKEGGAIHSKTVTLSNLKSTFTFADNTVKAIVESTPEAPEEIPPVEGEESTATEDPNSNTEGSSANTNLEGSQGDTADTGTGDVNNESQDTSDTGNAESEEQLQDSTQSNEENTLPNSNIDQSNENTDESSDSHTEEITDESVSSSSESGSSTPQDGGAASSGAPSGDQSISANACLAKSYAASTDSSPVSNSSGSEEPVTSSSDSDVTASSDNPDSSSSGDSAGDSEEPTEPEAGSTTETLTLIGGGAIYGETVKIENFSGQGIFSGNKAIDNTTEGSSSKSDVLGGAVYAKTLFNLDSGSSRRTVTFSGNTVSSQSTTGQVAGGAIYSPTVTIATPVVFSKNSATNNANNTTDTQRKDTFGGAIGATSAVSLSGGAHFLENVADLGSAIGLVPGTQNTETVKLESGSYYFEKNKALKRATIYAPVVSIKAYTATFNQNRSLEEGSAIYFTKEASIESLGSVLFTGNLVTLTLSTTTEGTPATTSGDVTKYGAAIFGQIASSNGSQTDNLPLKLIASGGNICFRNNEYRPTSSDTGTSTFCSIAGDVKLTMQAAKGKTISFFDAIRTSTKKTGTQATAYDTLDINKSEDSETVNSAFTGTILFSSELHENKSYIPQNVVLHSGSLVLKPNTELHVISFEQKEGSSLVMTPGSVLSNQTVADGALVINNMTIDLSSVEKNGIAEGNIFTPPELRIIDTTTGGSGGTPSTDSESNQNSDDTEEQNNNDASNQGESANGSSSPAVAAAHTSRTRNFAAAATATPTTTPTATTTTSNQVILGGEIKLIDPNGTFFQNPALRSDQQISLLVLPTDSSKMQAQKIVLTGDIAPQKGYTGTLTLDPDQLQNGTISVLWKFDSYRQWAYVPRDNHFYANSILGSQMLMVTVKQGLLNDKMNLARFEEVSYNNLWISGLGTMLSQVGTPTSEEFTYYSRGASVALDAKPAHDVIVGAAFSKMIGKTKSLKRENNYTHKGSEYSYQASVYGGKPFHFVINKKTEKSLPLLLQGVISYGYIKHDTVTHYPTIRERNKGEWEDLGWLTALRVSSVLRTPAQGDTKRITVYGELEYSSIRQKQFTETEYDPRYFDNCTYRNLAIPMGLAFEGELSGNDILMYNRFSVAYMLSIYRNSPTCKYQVLSSGEGGEIICGVPTRNSARGEYSTQLYLGPLWTLYGSYTIEADAHTLAHMMNCGARMTF

SEQ ID NO: 18 - protein, 1531 aa, Chlamydia trachomatis
MSSEKDIKSTCSKFSLSVVAAILASVSGLASCVDLHAGGQSVNELVYVGPQAVLLLDQIRDLFVGSKDSQAEGQYRLIVGDPSSFQEKDADTLPGKVEQSTLFSVTNPVVFQGVDQQDQVSSQGLICSFTSSNLDSPRDGESFLGIAFVGDSSKAGITLTDVKASLSGAALYSTEDLIFEKIKGGLEFASCSSLEQGGACAAQSILIHDCQGLQVKHCTTAVNAEGSSANDHLGFGGGAFFVTGSLSGEKSLYMPAGDMVVANCDGAISFEGNSANFANGGAIAASGKVLFVANDKKTSFIENRALSGGAIAASSDIAFQNCAELVFKGNCAIGTEDKGSLGGGAISSLGTVLLQGNHGITCDKNESASQGGAIFGKNCQISDNEGPVVFRDSTACLGGGAIAAQEIVSIQNNQAGISFEGGKASFGGGIACGSFSSAGGASVLGTIDISKNLGAISFSRTLCTTSDLGQMEYQGGGALFGENISLSENAGVLTFKDNIVKTFASNGKILGGGAILATGKVEITNNSEGISFTGNARAPQALPTQEEFPLFSKKEGRPLSSGYSGGGAILGREVAILHNAAVVFEQNRLQCSEEEATLLGCCGGGAVHGMDSTSIVGNSSVRFGNNYAMGQGVSGGALLSKTVQLAGNGSVDFSRNIASLGGGALQASEGNCELVDNGYVLFRDNRGRVYGGAISCLRGDVVISGNKGRVEFKDNIATRLYVEETVEKVEEVEPAPEQKDNNELSFLGRAEQSFITAANQALFASEDGDLSPESSISSEELAKRRECAGGAIFAKRVRIVDNQEAVVFSNNFSDIYGGAIFTGSLREEDKLDGQIPEVLISGNAGDVVFSGNSSKRDEHLPHTGGGAICTQNLTISQNTGNVLFYNNVACSGGAVRIEDHGNVLLEAFGGDIVFKGNSSFRAQGSDAIYFAGKESHITALNATEGHAIVFHDALVFENLEERKSAEVLLINSRENPGYTGSIRFLEAESKVPQCIHVQQGSLELLNGATLCSYGFKQDAGAKLVLAAGAKLKILDSGTPVQQGHAISKPEAEIESSSEPEGAHSLWIAKNAQTTVPMVDIHTISVDLASFSSSQQEGTVEAPQVIVPGGSYVRSGELNLELVNTTGTGYENHALLKNEAKVPLMSFVASGDEASAEISNLSVSDLQIHVVTPEIEEDTYGHMGDWSEAKIQDGTLVISWNPTGYRLDPQKAGALVFNALWEEGAVLSALKNARFAHNLTAQRMEFDYSTNVWGFAFGGFRTLSAENLVAIDGYKGAYGGASAGVDIQLMEDFVLGVSGAAFLGKMDSQKFDAEVSRKGVVGSVYTGFLAGSWFFKGQYSLGETQNDMKTRYGVLGESSASWTSRGVLADALVEYRSLVGPVRPTFYALHFNPYVEVSYASMKFPGFTEQGREARSFEDASLTNITIPLGMKFELAFIKGQFSEVNSLGISYAWEAYRKVEGGAVQLLEAGFDWEGAPMDLPRQELRVALENNTEWSSYFSTVLGLTAFCGGFTSTDSKLGYEANTGLRLIF

SEQ ID NO: 19 - protein, 964 aa, Chlamydia trachomatis
MKKAFFFFLIGNSLSGLAREVPSRIFLMPNSVPDPTKESLSNKISLTGDTHNLTNCYLDNLRYILAILQKTPNEGAAVTITDYLSFFDTQKEGIYFAKNLTPESGGAIGYASPNSPTVEIRDTIGPVIFENNTCCRLFTWRNPYAADKIREGGAIHAQNLYINHNHDVVGFMKNFSYVQGGAISTANTFVVSENQSCFLFMDNICIQTNTAGKGGAIYAGTSNSFESNNCDLFFINNACCAGGAIFSPICSLTGNRGNIVFYNNRCFKNVETASSEASDGGAIKVTTRLDVTGNRGRIFFSDNITKNYGGAIYAPVVTLVDNGPTYFINNIANNKGGAIYIDGTSNSKISADRHAIIFNENIVTNVTNANGTSTSANPPRRNAITVASSSGEILLGAGSSQNLIFYDPIEVSNAGVSVSFNKEADQTGSVVFSGATVNSADFHQRNLQTKTPAPLTLSNGFLCIEDHAQLTVNRFTQTGGVVSLGNGAVLSCYKNGTGDSASNASITLKHIGLNLSSILKSGAEIPLLWVEPTNNSNNYTADTAATFSLSDVKLSLIDDYGNSPYESTDLTHALSSQPMLSISEASDNQLQSENIDFSGLNVPHYGWQGLWTWGWAKTQDPEPASSATITDPQKANRFHRTLLLTWLPAGYVPSPKHRSPLIANTLWGNMLLATESLKNSAELTPSGHPFWGITGGGLGMMVYQDPRENHPGFHMRSSGYSAGMIAGQTHTFSLKFSQTYTKLNERYAKNNVSSKNYSCQGEMLFSLQEGFLLTKLVGLYSYGDHNCHHFYTQGENLTSQGTFRSQTMGGAVFFDLPMKPFGSTHILTAPFLGALGIYSSLSHFTEVGAYPRSFSTKTPLINVLVPIGVKGSFMNATHRPQAWTVELAYQPVLYRQEPGIAAQLLASKGIWFGSGSPSSRHAMSYKISQQTQPLSWLTLHFQYHGFYSSSTFCNYLNGEIALRF

SEQ ID NO: 20 - protein, 1034 aa, Chlamydia trachomatis
MIKRTSLSFACLSFFYLSTISILQANETDTLQFRRFTFSDREIQFVLDPASLITAQNIVLSNLQSNGTGACTISGNTQTQIFSNSVNTTADSGGAFDMVTTSFTASDNANLLFCNNYCTHNKGGGAIRSGGPIRFLNNQDVLFYNNISAGAKYVGTGDHNEKNRGGALYATTITLTGNRTLAFINNMSGDCGGAISADTQISITDTVKGILFENNHTLNHIPYTQAENMARGGAICSRRDLCSISNNSGPIVFNYNQGGKGGAISATRCVIDNNKERIIFSNNSSLGWSQSSSASNGGAIQTTQGFTLRNNKGSIYFDSNTATHAGGAINCGYIDIRDNGPVYFLNNSAAWGAAFNLSKPRSATNYIHTGTGDIVFNNNVVFTLDGNLLGKRKLFHINNNEITPYTLSLGAKKDTRIYFYDLFQWERVKENTSNNPPSPTSRNTITVNPETEFSGAVVFSYNQMSSDIRTLMGKEHNYIKEAPTTLKFGTLAIEDDAELEIFNIPFTQNPTSLLALGSGATLTVGKHGKLNITNLGVILPIILKEGKSPPCIRVNPQDMTQNTGTGQTPSSTSSISTPMIIFNGRLSIVDENYESVYDSMDLSRGKAEQLILSIETTNDGQLDSNWQSSLNTSLLSPPHYGYQGLWTPNWITTTYTITLNNNSSAPTSATSIAEQKKTSETFTPSNTTTASIPNIKASAGSGSGSASNSGEVTITKHTLVVNWAPVGYIVDPIRRGDLIANSLVHSGRNMTMGLRSLLPDNSWFALQGAATTLFTKQQKRLSYHGYSSASKGYTVSSQASGAHGHKFLLSFSQSSDKMKEKETNNRLSSRYYLSALCFEHPMFDRIALIGAAACNYGTHNMRSFYGTKKSSKGKFHSTTLGASLRCELRDSMPLRSIMLTPFAQALFSRTEPASIRESGDLARLFTLEQAHTAVVSPIGIKGAYSSDTWPTLSWEMELAYQPTLYWKRPLLNTLLIQNNGSWVTTNTPLAKHSFYGRGSHSLKFSHLKLFANYQAEVATSTVSHYINAGGALVF

SEQ ID NO: 21 - protein, 1013 aa, Chlamydia trachomatis
MQTSFHKFFLSMILAYSCCSLSGGGYAAEIMIPQGIYDGETLTVSFPYTVIGDPSGTTVFSAGELTLKNLDNSIAALPLSCFGNLLGSFTVLGRGHSLTFENIRTSTNGAALSDSANSGLFTIEGFKELSFSNCNSLLAVLPAATTNNGSQTPTTTSTPSNGTIYSKTDLLLLNNEKFSFYSNLVSGDGGAIDAKSLTVQGISKLCVFQENTAQADGGACQVVTSFSAMANEAPIAFIANVAGVRGGGIAAVQDGQQGVSSSTSTEDPVVSFSRNTAVEFDGNVARVGGGIYSYGNVAFLNNGKTLFLNNVASPVYIAAEQPTNGQASNTSDNYGDGGAIFCKNGAQAAGSNNSGSVSFDGEGVVFFSSNVAAGKGGAIYAKKLSVANCGPVQFLGNIANDGGAIYLGESGELSLSADYGDIIFDGNLKRTAKENAADVNGVTVSSQAISMGSGGKITTLRAKAGHQILFNDPIEMANGNNQPAQSSEPLKINDGEGYTGDIVFANGNSTLYQNVTIEQGRIVLREKAKLSVNSLSQTGGSLYMEAGSTLDFVTPQPPQQPPAANQLITLSNLHLSLSSLLANNAVTNPPTNPPAQDSHPAIIGSTTAGSVTISGPIFFEDLDDTAYDRYDWLGSNQKIDVLKLQLGTQPSANAPSDLTLGNEMPKYGYQGSWKLAWDPNTANNGPYTLKATWTKTGYNPGPERVASLVPNSLWGSILDIRSAHSAIQASVDGRSYCRGLWVSGVSNFFYHDRDALGQGYRYISGGYSLGANSYFGSSMFGLAFTEVFGRSKDYVVCRSNHHACIGSVYLSTKQALCGSYLFGDAFIRASYGFGNQHMKTSYTFAEESDVRWDNNCLVGEIGVGLPIVITPSKLYLNELRPFVQAEFSYADHESFTEEGDQARAFRSGHLMNLSVPVGVKFDRCSSTHPNKYSFMGAYICDAYRTISGTQTTLLSHQETWTTDAFHLARHGVIVRGSMYASLTSNIEVYGHGRYEYRDTSRGYGLSAGSKVRF

SEQ ID NO: 22 - protein, 1016 aa, Chlamydia trachomatis
MPFSLRSTSFCFLACLCSYSYGFASSPQVLTPNVTTPFKGDDVYLNGDCAFVNVYAGAENGSIISANGDNLTITGQNHTLSFTDSQGPVLQNYAFISAGETLTLKDFSSLMFSKNVSCGEKGMISGKTVSISGAGEVIFWDNSVGYSPLSIVPASTPTPPAPAPAPAASSSLSPTVSDARKGSIFSVETSLEISGVKKGVMFDNNAGNFGTVFRGNSNNNAGSGGSGSATTPSFTVKNCKGKVSFTDNVASCGGGVVYKGTVLFKDNEGGIFFRGNTAYDDLGILAATSRDQNTETGGGGGVICSPDDSVKFEGNKGSIVFDYNFAKGRGGSILTKEFSLVADDSVVFSNNTAEKGGGAIYAPTIDISTNGGSILFERNRAAEGGAICVSEASSGSTGNLTLSASDGDIVFSGNMTSDRPGERSAARILSDGTTVSLNASGLSKLIFYDPVVQNNSAAGASTPSPSSSSMPGAVTINQSGNGSVIFTAESLTPSEKLQVLNSTSNFPGALTVSGGELVVTEGATLTTGTITATSGRVTLGSGASLSAVAGAANNNYTCTVSKLGIDLESFLTPNYKTAILGADGTVTVNSGSTLDLVMESEAEVYDNPLFVGSLTIPFVTLSSSSASNGVTKNSVTINDADAAHYGYQGSWSADWTKPPLAPDAKGMVPPNTNNTLYLTWRPASNYGEYRLDPQRKGELVPNSLWVAGSALRTFTNGLKEHYVSRDVGFVASLHALGDYILNYTQDDRDGFLARYGGFQATAASHYENGSIFGVAFGQLYGQTKSRMYYSKDAGNMTMLSCFGRSYVDIKGTETVMYWETAYGYSVHRMHTQYFNDKTQKFDHSKCHWHNNNYYAFVGAEHNFLEYCIPTRQFARDYELTGFMRFEMAGGWSSSTRETGSLTRYFARGSGHNMSLPIGIVAHAVSHVRRSPPSKLTLNMGYRPDIWRVTPHCNMEIIANGVKTPIQGSPLARHAFFLEVHDTLYIHHFGRAYMNYSLDARRRQTAHFVSMGLNRIF

SEQ ID NO: 23 - protein, 878 aa, Chlamydia trachomatis
MRPDHMNFCCLCAAILSSTAVLFGQDPLGETALLTKNPNHVVCTFFEDCTMESLFPALCAHASQDDPLYVLGNSYCWFVSKLHITDPKEALFKEKGDLSIQNFRFLSFTDCSSKESSPSIIHQKNGQLSLRNNGSMSFCRNHAEGSGGAISADAFSLQHNYLFTAFEENSSKGNGGAIQAQTFSLSRNVSPISFARNRADLNGGAICCSNLICSGNVNPLFFTGNSATNGGAICCISDLNTSEKGSLSLACNQETLFASNSAKEKGGAIYAKHMVLRYNGPVSFINNSAKIGGAIAIQSGGSLSILAGEGSVLFQNNSQRTSDQGLVRNAIYLEKDAILSSLEARNGDILFFDPIVQESSSKESPLPSSLQASVTSPTPATASPLVIQTSANRSVIFSSERLSEEEKTPDNLTSQLQQPIELKSGRLVLKDRAVLSAPSLSQDPQALLIMEAGTSLKTSSDLKLATLSIPLHSLDTEKSVTIHAPNLSIQKIFLSNSGDENFYENVELLSKEQNNIPLLTLSKEQSHLHLPDGNLSSHFGYQGDWTFSWKDSDEGHSLIANWTPKNYVPHPERQSTLVANTLWNTYSDMQAVQSMINTIAHGGAYLFGTWGSAVSNLFYAHDSSGKPIDNWHHRSLGYLFGISTHSLDDHSFCLAAGQLLGKSSDSFITSTETTSYIATVQAQLATPLMKISAQACYNESIHELKTKYRSFSKEGFGSWHSVAVSGEVCASIPIVSNGSGLFSSFSIFSKLQGFSGTQDGFEESSGEIRSFSASSFRNISLPMGITFEKKSQKTRNYYYFLGAYIQDLKRDVESGPVVLLKNAVSWDAPMANLDSRAYMFRLTNQRALHRLQTLLNVSYVLRGQSHSYSLDLGTTYRF

SEQ ID NO: 24 - protein, 393 aa, Chlamydia trachomatis
MKKLLKSVLVFAALSSASSLQALPVGNPAEPSLMIDGILWEGFGGDPCDPCATWCDAISMRVGYYGDFVFDRVLKTDVNKEFQMGAKPTTDTGNSAAPSTLTARENPAYGRHMQDAEMFTNAACMALNIWDRFDVFCTLGATSGYLKGNSASFNLVGLFGDNENQKTVKAESVPNMSFDQSVVELYTDTTFAWSVGARAALWECGCATLGASFQYAQSKPKVEELNVLCNAAEFTINKPKGYVGKEFPLDLTAGTDAATGTKDASIDYHEWQASLALSYRLNMFTPYIGVKWSRASFDADTIRIAQPKSATAIFDTTTLNPTIAGAGDVKTGAEGQLGDTMQIVSLQLNKMKSRKSCGIAVGTTIVDADKYAVTVETRLIDERAAHVNAQFRF

SEQ ID NO: 25 - protein, 10 aa, Chlamydia trachomatis
CSFIGGITYL

SEQ ID NO: 26 - protein, 9 aa, Chlamydia trachomatis
SFIGGITYL

SEQ ID NO: 27 - protein, 9 aa, Chlamydia trachomatis
SIIGGITYL

SEQ ID NO: 28 - protein, 298 aa, Chlamydia trachomatis
MASICGRLGSGTGNALKAFFTQPNNKMARVVNKTKGMDKTIKVAKSAAELTANILEQAGGAGSSAHITASQVSKGLGDARTVVALGNAFNGALPGTVQSAQSFFSHMKAASQKTQEGDEGLTADLCVSHKRRAAAAVCSIIGGITYLATFGAIRPILFVNKMLAKPFLSSQTKANMGSSVSYIMAANHAASVVGAGLAISAERADCEARCARIAREESLLEVPGEENACEKKVAGEKAKTFTRIKYALLTMLEKFLECVADVFKLVPLPITMGIRAIVAAGCTFTSAIIGLCTFCARA

SEQ ID NO: 29 - protein, 544 aa, Chlamydia trachomatis
MVAKNIKYNEEARKKIQKGVKTLAEAVKVTLGPKGRHVVIDKSFGSPQVTKDGVTVAKEVELADKHENMGAQMVKEVASKTADKAGDGTTTATVLAEAIYTEGLRNVTAGANPMDLKRGIDKAVKVVVDQIRKISKPVQHHKEIAQVATISANNDAEIGNLIAEAMEKVGKNGSITVEEAKGFETVLDIVEGMNFNRGYLSSYFATNPETQECVLEDALVLIYDKKISGIKDFLPVLQQVAESGRPLLIIAEDIEGEALATLVVNRIRGGFRVCAVKAPGFGDRRKAMLEDIAILTGGQLISEELGMKLENANLAMLGKAKKVIVSKEDTTIVEGMGEKEALEARCESIKKQIEDSSSDYDKEKLQERLAKLSGGVAVIRVGAATEIEMKEKKDRVDDAQHATIAAVEEGILPGGGTALIRCIPTLEAFLPMLTNEDEQIGARIVLKALSAPLKQIAANAGKEGAIIFQQVMSRSANEGYDALRDAYTDMLEAGILDPAKVTRSALESAASVAGLLLTTEALIAEIPEEKPAAAPAMPGAGMDY

SEQ ID NO: 30 - protein, 553 aa, Chlamydia trachomatis
MRIGDPMNKLIRRAVTIFAVTSVASLFASGVLETSMAESLSTNVISLADTKAKDNTSHKSKKARKNHSKETPVDRKEVAPVHESKATGPKQDSCFGRMYTVKVNDDRNVEITQAVPEYATVGSPYPIEITATGKRDCVDVIITQQLPCEAEFVRSDPATTPTADGKLVWKIDRLGQGEKSKITVWVKPLKEGCCFTAATVCACPEIRSVTKCGQPAICVKQEGPENACLRCPVVYKINIVNQGTATARNVVVENPVPDGYAHSSGQRVLTFTLGDMQPGEHRTITVEFCPLKRGRATNIATVSYCGGHKNTASVTTVINEPCVQVSIAGADWSYVCKPVEYVISVSNPGDLVLRDVVVEDTLSPGVTVLEAAGAQISCNKVVWTVKELNPGESLQYKVLVRAQTPGQFTNNVVVKSCSDCGTCTSCAEATTYWKGVAATHMCVVDTCDPVCVGENTVYRICVTNRGSAEDTNVSLMLKFSKELQPVSFSGPTKGTITGNTVVFDSLPRLGSKETVEFSVTLKAVSAGDARGEAILSSDTLTVPVSDTENTHIY

SEQ ID NO: 31 - protein, 326 aa, Chlamydia trachomatis
MFRYTLSRSLFFILALFFCSACDSRSMITHGLSGRDANEIVVLLVSKGVAAQKVPQAASSTGGSGEQLWDISVPAAQITEALAILNQAGLPRMKGTSLLDLFAKQGLVPSEMQEKIRYQEGLSEQMATTIRKMDGIVDASVQISFSPEEEDQRPLTASVYIKHRGVLDNPNSIMVSKIKRLVASAVPGLCPENVSVVSDRASYSDITINGPWGLSDEMNYVSVWGIILAKHSLTKFRLVFYFLILLLFILSCGLLWVIWKTHTLISALGGTKGFFDPAPYSQLSFTQNKPAPKETPGAAEGAEAQTASEQPSKENAEKQEENNEDA

SEQ ID NO: 32 - protein, 188 aa, Chlamydia trachomatis
MRKTIFKAFNLLFSLLFLSSCSYPCRDWECHGCDSARPRKSSFGFVPFYSDEEIQQAFVEDFDSKEEQLYKTSAQSTSFRNITFATDSYSIKGEDNLTILASLVRHLHKSPKATLYIEGHTDERGAAAYNLALGARRANAVKQYLIKQGIAADRLFTISYGKEHPVHPGHNELAWQQNRRTEFKIHAR

SEQ ID NO: 33 - protein, 243 aa, Chlamydia trachomatis
MKNILSWMLMFAVALPIVGCDNGGGSQTSATEKSMVEDSALTDNQKLSRTFGHLLSRQLSRTEDFSLDLVEVIKGMQSEIDGQSAPLTDTEYEKQMAEVQKASFEAKCSENLASAEKFLKENKEKAGVIELEPNKLQYRVVKEGTGRVLSGKPTALLHYTGSFIDGKVFDSSEKNKEPILLPLTKVIPGFSQGMQGMKEGEVRVLYIHPDLAYGTAGQLPPNSLLIFEVKLIEANDDNVSVTE

SEQ ID NO: 34 - protein, 432 aa, Chlamydia trachomatis
MKKYFYKGFVGALLLACGSTNLAFAQASSMDSQLWSVEDLDSYLSSKGFVETRKRDGVLRLAGDVRARWIYAKEDLETTQTPAKPMLPTNRYRSEFNLYVDYTAANSWMTSKMNWVTIAGGESSAAGLDINRAFLGYRFYKNPETQAEVFAEIGRSGLGDIFDSDVQFNSNFDGIHLYAARRISEKLPFTMIVHGGPFVVNMAEKEYAWVVEAILNKLPGNFVVKTSVVDWNTLTAKTNDPADASAAQPAKPNTKYDYLVWQWLVGKSTAMPWFNGQTKNLYTYGAYLFNPLAEIPENWKQSTTPTTKITNGKENHAWFIGCSLGGVRRAGDWSATVRYEYVEALAIPEIDVAGIGRGNQMKYWFAQAIKQGLDPKESNGFTNYKGVSYQFVMGLTDSVSFRAYAAYSKPANDNLGSDFTYRKYDLGLISSF

SEQ ID NO: 35 - protein, 441 aa, Chlamydia trachomatis
MWLIVASTLLACLAMALVFKAYRHVISFRSYVNQVIRDVRLSVDLKEWAVAEMRLAPILKKRQYRRKYLFEYIRILRELERFEEAEKLLGEAKKLKLAGAHFFLEVAHKAFRHGAYKEAAHAFSLLSAELMGEREVARYTISLVYLGEVDAACRIIEPWIGPLAHQEVFISVGHIYFATKRYADAIDFYRRARSLGSCPIDVLYNLAHSLRICGQYVDAGMLFRELLGDPVYKDEAMFNIGLCEQKLGNSKKALLIYQNSELWVRGDALMMRYAALAAADQQDYQLAEHCWTLAFRCQSYADDWNCCVHYGLALCHLKKYAEAEKVYLRVIQKTPDCLVACKALAWLAGVGHATMISAREGIAYAKRALQIKRSPEVLELLSACEAREGNFDVAYDIQAILAERDTTAKERERRSQILKNLRQKLPIDQQHIVEVSLLLAA

SEQ ID NO: 36 - protein, 393 aa, Chlamydia trachomatis
MLVESQLGLEDVLEAFSERNFDIQSKSFIESFQDKKLRRTVIQRFLHHPLLHIHDIARAAYLLAALEEGVDLGYQFLCMHQTQSGAALLFRRAGFLWGGLPYPGEHAEMAMLLSRIAEFYDTSYEQVQKMIAFQHALFSHERNIFPALWSQEGSRSNQEKTAVSKLLFCQKEARIEDQFTLTDMSLGFWMRRTPSFSAYVSGSGCKSGVGAFLIGDVGVLNYGPCVGDPGECLGFGLCGQVKEFSCQEKDEEVSISFAGALSQPSSRRTGFSYLQDALFSTNSCYCIDITEQKCHVASSLDRENQDAFFAIFCKGSQCQVCNGPKLRTGSPDSYKGPAYDVLIKGEKETVRILSSSPHMEIFSLQGKDRFWGSNFLINLPYTQNSINILFEKA

SEQ ID NO: 37 - protein, 316 aa, Chlamydia trachomatis
MGKFFASYLLILAPFFLQSCSAPSRTTLEGVRMTIPYRIVFGEALSPDAFQQAQKEIDRVFDHIDQTFNNWNPLSEISRINRTTKQTPIPLSPALFAFLCEIDHFHAFSDGRFDPTLGALKSLWLLHLKSHTIPSQELQHLYKHSSGWHLISLDKTQQTLRKLSPLVQLDLCGTVKGFAVDLLGTACAQFCQNYYVEWGGEIKTKGKHPSGRSWAVASSATPEILHLHDHAIATSGSQYQRWHVDNKTYTHILDPLTGTPLEDSSHPILAVSVINESCAFADAMATALTTFSSKQEALDWANKKHLCAYITDKNVS

SEQ ID NO: 38 - protein, 1005 aa, Chlamydia trachomatis
MTNSISGYQPTVTTSTSSTTSASGASGSLGASSVSTTANATVTQTANATNSAATSSIQTTGETVVNYTNSASAPNVTVSTSSSSTQATATSNKTSQAVAGKITSPDTSESSETSSTSSSDHIPSDYDDVGSNSGDISNNYDDVGSNNGDISSNYDDAAADYEPIRTTENIYESIGGSRTSGPENTSGGAAAALNSLRGSSYSNYDDAAADYEPIRTTENIYESIGGSRTSGPENTSGGAAAALNSLRGSSYSNYDDAAADYEPIRTTENIYESIGGSRTSGPENTSDGAAAAALNSLRGSSYTTGPRNEGVFGPGPEGLPDMSLPSYDPTNKTSLLTFLSNPHVKSKMLENSGHFVFIDTDRSSFILVPNGNWDQVCSIKVQNGKTKEDLDIKDLENMCAKFCTGFSKFSGDWDSLVEPMVSAKAGVASGGNLPNTVIINNKFKTCVAYGPWNSQEASSGYTPSAWRRGHRVDFGGIFEKANDFNKINWGTQAGPSSEDDGISFSNETPGAGPAAAPSPTPSSIPIINVNVNVGGTNVNIGDTNVNTTNTTPTTQSTDASTDTSDIDDINTNNQTDDINTTDKDSDGAGGVNGDISETESSSGDDSGSVSSSESDKNASVGNDGPAMKDILSAVRKHLDVVYPGENGGSTEGPLPANQTLGDVISDVENKGSAQDTKLSGNTGAGDDDPTTTAAVGNGAEEITLSDTDSGIGDDVSDTASSSGDESGGVSSPSSESNKNTAVGNDGPSGLDILAAVRKHLDKVYPGDNGGSTEGPLQANQTLGDIVQDMETTGTSQETVVSPWKGSTSSTESAGGSGSVQTLLPSPPPTPSTTTLRTGTGATTTSLMMGGPIKADIITTGGGGRIPGGGTLEKLLPRIRAHLDISFDAQGDLVSTEEPQLGSIVNKFRQETGSRGILAFVESAPGKPGSAQVLTGTGGDKGNLFQAAAAVTQALGNVAGKVNLAIQGQKLSSLVNDDGKGSVGRDLFQAAAQTTQVLSALIDTVG

SEQ ID NO: 39 - protein, 148 aa, Chlamydia trachomatis
MFQPETVPSNRSTETTPQNIEVYNDRNFTNHTTEDVIRIGERLQRQFYNMTEESRVPFTTSPSHHTGNWKTAFLYNLSQVVAHIFPSTVQPIRVKPTRIPPSPTPPPEGTTTAETSTSENKVTTISKEQEVTTKPLLVRERRSLLHSQ

SEQ ID NO: 40 - protein, 340 aa, Chlamydia trachomatis
MSSKLVNYLRLTFLSFLGIASTSLDAMPAGNPAFPVIPGINIEQKNACSFDLCNSYDVLSALSGNLKLCFCGDYIFSEEAQVKDVPVVTSVTTAGVGPSPDITSTTKTRNFDLVNCNLNTNCVAVAFSLPDRSLSAIPLFDVSFEVKVGGLKQYYRLPMNAYRDFTSEPLNSESEVTDGMIEVQSNYGFVWDVSLKKVIWKDGVSFVGVGADYRHASCPIDYIIANSQANPEVFIADSDGKLNFKEWSVCVGLTTYVNDYVLPYLAFSIGSVSRQAPDDSFKKLEDRFTNLKFKVRKITSSHRGNICIGATNYVADNFFYNVEGRWGSQRAVNVSGGFQF

SEQ ID NO: 41 - protein, 560 aa, Chlamydia trachomatis
MSISGSGNVSPATPDFDPSILMGRQAASAHAAKEASGASKATETSAAEQQALISSGTELDYVTDLQQSEGKYKKTLDKTSKSPKTKLKGNFSKVRAGTKGFLTGFGTRASRISARKAENNGEGMSMIPSQMEYVKKKGNRVSPEMQNFYLGASGLWSPTSDVSSITENCLGATALSTTPLLTTMQDPVSIEHLSSGEITALASFNPNVRTASLNEQTINAWTEARLGGEMVSTLLDPNIETSSLLRRAPTVSNEGMVDVSDMGNQTTSLSMEGLVNTVVDDPASAEEEKKTGELSLEEMAAMAKMMAALLSSGQGMAVFIASSTPSSGLTQFPEPKFSGTIPHHFSKKEDNETIWGLDSQIGSIAFDTRRENNASPLPTTSLHEEASYRFPVGEAPLDVNEIPFAVQHSTVFSKETANTEQALIQNESLGEIPVSAEVVGQDTVSSAYQFPSHLGMAVLASVPLSTEDYKTAVEHRKGPGGPPDPLIYQYRNVAVDPAIIFQSPSPFSVSSRFSVQGKPEAVAVYNDDQEEAAGGNRDSDEGKDQEQDKTRETEDAGGDS

SEQ ID NO: 42 - protein, 236 aa, Chlamydia trachomatis
MLSKFCKLSLSAILLINTLAPSETFSEEGTSGFLGRMKSWILKDKTILSTTEESQTSAIEKVSDLLSWKRYDYTQESGFAIQFPESPEHSEQVIEVPQSDLAIRYDTYVAETPSDSTVYVVSIWEYPEKIDISRPELNLQEGFAGMLYALPESQVLYLKATALQGHKALEFWIACDDVYFRGMLVSVNHTLYQVFMVYKGRSPEILDKEYSTFIQSFKVTKVRNSKKMDIRKRVSL

SEQ ID NO: 43 - protein, 536 aa, Chlamydia trachomatis
MNDTKNNISSSFWNPNKVVTKVLLKVSETGIESTPGIVKHNQLITQSENPTDPTDAVTFKYLKENYTKENDPNPGFLPTTGGTMTGDIDMQGNNVTDIVMYTNGQQNPTDDSAVTIGYLNEKADEIKSNDQITTAVAGLSNINSQISTLHQLLGIAEDPDTVTNPDLLKTSGGTVYEDIDMSSNTVSDLGTPTNKDTKSAINVEFVQAKITSPQMAFLKNNDTNLSNITVSEYFNWLQDPTQAPTPEPDPDPEPAPEPEPDTSDSSGSGSENPADPAPTNPSDSNAQNNPTPSSNGATASIRKLAATTTTVPTDTEIAPAAEDPNLPNTTFSEKSPLWEEFFSFSDSSRSEMVIQKTGILTFSMQGTWENPSSSQTPSTDPISLELTVTPPTTDTPPESPPSPPEAPAPEATPSPTNNNLTASITKTFSRKYNLSATPSPTPTTPTEPTTITKTLSLSSGQSCTLQIPVQATRSVLKLKYVNPNNNSSGGSSGSGGSSQPETTPTGITLQSFSWSLVLTPGEITKATSTPSTPSQP

SEQ ID NO: 44 - protein, 404 aa, Chlamydia trachomatis
MSVQGSSSLKYSDLFKPPEPTSSTDSSKEPPKESAWKVVSHSRGRRRARSNPSPHTSQNTPSPKDSSLVARTDKAATDIFNSAKHKAIETTKRSDQQSRSLHILHLLAENPEPIVFHSAHQTNHNDPQRMLCDAILQANRIITMRIFNIGSPEIIRALIRAVRRNIPVVVSAWNFPNLSNWDRESELCVELRGNPQICLHKKTTLIDNQLTIIGTANYTKSSFFKDINLTALIQNPALYSLILSDTRGSVSIGSQTISYYPLPFPQSNTKILPIIQEIQKAQRTIKIAMNIFSHTEIFLALEQARLRGVTITIVINKKESAHTLDILHRISALLLLKSVTTVDSLHAKICLIDNQTLIFGSPNWTYHGMHKNLEDLLIVTPLTPKQIHSIQEIWAFLLKNSSPV

SEQ ID NO: 45 - protein, 245 aa, Chlamydia trachomatis
MDRSPLFLIIMGAPGSGKGTQSKLLASQLSLLHISSGDLLRDAVSKDTPLSQEIKSYLDQGKLLPDTLVWKLVHEKLDEFQQDTLLRRLSFLSRSENSAILDGFPRTVTQAKLLHEFLSSYFPNYKVILLDISDEEVLNRLTSRYICPACQGIYNEQQGFSSCPKCSVELIRRSDDTLEVILDRIQTYKQETQPVLDYYTEKQKLITIDANAPTQQVFQSILDSLSASLVYQERDCCNCDCDDED

SEQ ID NO: 46 - protein, 810 aa, Chlamydia trachomatis
MTKPSFLYVIQPFSVFNPRLGRFSTDSDTYIEEENRLASFIESLPLEIFDIPSFMETAISNSPYILSWETTKDGALFTILEPKLSACAATCLVAPSIQMKSDAELLEEIKQALLRSSHDGVKYRITRESFSPEKKTPKVALVDDDIELIRNVDFLGRAVDIVKLDPINILNTVSEENILDYSFTRETAQLSADGRFGIPPGTKLFPKPSFDVEISTSIFEETTSFTRSFSASVTFSVPDLAATMPLQSPPMVENGQKEICVIQKHLFPSYSPKLVDIVKRYKREAKILINKLAFGMLWRHRAKSQILTEGSVRLDLQGFTESKYNYQIQVGSHTIAAVLIDMDISKIQSKSEQAYAIRKIKSGFQRSLDDYHIYQIERKQTFSFSPKHRSLSSTSHSEDSDLDLSEAAAFSGSLTCEFVKKSTQHAKNTVTCSTAAHSLYTLKEDDSSNPSEKRLDSCFRNWIENKLSANSPDSWSAFIQKFGTHYIASATFGGIGFQVLKLSFEQVEDLHSKKISLETAAANSLLKGSVSSSTESGYSSYSSTSSSHTVFLGGTVLPSVHDERLDFKDWSESVHLEPVPIQVSLQPITNLLVPLHFPNIGAAELSNKRESLQQAIRVYLKEHKVDEQGERTTFTSGIDNPSSWFTLEAAHSPLIVSTPYIASWSTLPYLFPTLRERSSATPIVFYFCVDNNEHASQKILNQSYCFLGSLPIRQKIFGSEFASFPYLSFYGNAKEAYFDNTYYPTRCGWIVEKLNTTQDQFLRDGDEVRLKHVSSGKYLATTPLKDTHGTLTRTTNCEDAIFIIKKSSGY

SEQ ID NO: 47 - protein, 173 aa, Chlamydia trachomatis; identical to SEQ ID NO: 6

SEQ ID NO: 48 - protein, 194 aa, Chlamydia trachomatis
MFKRPAKNFFDEVQTLYEDSGANSTSYSIYPQRTERLENHSNIFEPAKPAETRLLSQEEHSQWTDQQEELATQESSFPEEPETTLGEGVSFKGELTFERLLRIDGTFEGILVSKGKIIVGPQGYVKANIELEEAVIAGVVEGNITVTGRVSLQGRAMVTGDIQAGSLCVDEGVRLCGYVSIQGAPSNEQEEIDS

SEQ ID NO: 49 - protein, 155 aa, Chlamydia trachomatis
MRAVLHLEHKRYFQNHGHILFEGLAPVSDCKQLEAELKLFLKEVAVVKDRHLQRWRENVHRTLPEVQMIVKRVRLDHLAAELTHRSRVALVRDLWVQKQEEIFFDDCDCSVLLCLSGEKAGWGLFFSGEYPQDVFNWGAGDTAIILRFSSAGFPN

SEQ ID NO: 50 - protein, 442 aa, Chlamydia trachomatis
MQAAHHHYHRYTDKLHRQNHKKDLISPKPTEQEACNTSSLSKELIPLSEQRGLLSPICDFISERPCLHGVSVRNLKQALKNSAGTQIALDWSILPQWFNPRVSHAPKLSIRDFGYSAHQTVTEATPPCWQNCFNPSAAVTIYDSSYGKGVFQISYTLVRYWRENAATAGDAMMLAGSINDYPSRQNIFSQFTFSQNFPNERVSLTIGQYSLYAIDGTLYNNDQQLGFISYALSQNPTATYSSGSLGAYLQVAPTASTSLQIGFQDAYNISGSSIKWSNLTKNRYNFHGFASWAPRCCLGSGQYSVLLYVTRQVPEQMEQTMGWSVNASQHISSKLYVFGRYSGVTGHVFPINRTYSFGMASANLFNRNPQDLFGIACAFNNVHLSASPNTKRKYETVIEGFATIGCGPYLSFAPDFQLYLYPALRPNKQSARVYSVRANLAI

SEQ ID NO: 51 - protein, 975 aa, Chlamydia trachomatis; identical to SEQ ID NO: 15

SEQ ID NO: 52 - protein, 696 aa, Chlamydia trachomatis
MIDKIIRTILVLSLFLLYWSSDLLEKDVKSIKRELKALHEDVLELVRISHQQKNWVQSTDFSVSPEISVLKDCGDPAFPNLLCEDPYVEKVVPSLLKEGFVPKGILRTAQVGRPDNLSPFNGFVNIVRFYELCVPNLAVEHVGKYEEFAPSLALKIEEHYVEDGSGDKEFHIYLRPNMFWEPIDPTLFPKNITLADSFLRPHPVTAHDVKFYYDVVMNPYVAEMRAVAMRSYFEDMVSVRVENDLKLIVRWRAHTVRNEQGEEEKKVLYSAFANTLALQPLPCFVYQHFANGEKIVPEDSDPDTYRKDSVWAQNFSSHWAYNYIVSCGAFRFAGMDDEKITLVRNPNYHNPFAALVEKRYIYMKDSTDSLFQDFKAGKVDIAYFPPNHVDNLASFMQTSAYKEQAARGEAILEKNSSDRSYSYIGWNCLSLFFNNRSVRQAMNMLIDRDRIIEQCLDGRGVSVSGPFSLCSPSYNRDVEGWQYSPEEAARKLEEEGWIDADGDGIREKVIDGVVVPFRFRLCYYVKSVTARTIAEYVATVCKEVGIECCLLGLDMADYSQALEEKNFDAILSGWCLGTPPEDPRALWHSEGALEKGSANAVGFCNEEADRIIEQLSYEYDSNKRQALYHRFHEVIHEESPYAFLYSRQYSLVYKEFVKNIFVPTEHQDLIPGAQDETVNLSMLWVDKEEGRCSAIS

SEQ ID NO: 53 - protein, 194 aa, Chlamydia trachomatis
MLKMFWLNSLVFFSLLLSACGYTVLSPHYVEKKFSLSEGIYVCPIEGDSLGDLVSSLSYELEKRGLHTRSQGTSSGYVLKVSLFNETDENIGFAYTPQKPDEKPVKHFIVSNEGRLALSAKVQLIKNRTQEILVEKCLRKSVTFDFQPDLGTANAHQLALGQFEMHNEAIKSASRILYSQLAETIVQQVYYDLF

SEQ ID NO: 54 - protein, 167 aa, Chlamydia trachomatis
MSRQNAEENLKNFAKELKLPDVAFDQNNTCILFVDGEFSLHLTYEEHSDRLYVYAPLLDGLPDNPQRRLALYEKLLEGSMLGGQMAGGGVGVATKEQLILMHCVLDMKYAETNLLKAFAQLFIETVVKWRTVCSDISAGREPTVDTMPQMPQGGGGGIQPPPAGIRA

SEQ ID NO: 55 - protein, 144 aa, Chlamydia trachomatis
MKNNSAQKIIDSIKQILSIYKIDFEPSFGATLTDDNDLDYQMLIEKTQEKIQELDKRSQEILQQTGMTREQMEVFANNPDNFSPEEWRALENIRSSCNEYKKETEELIKEVTNDIGHSSHKSPTPKKTKSSSQKKSKKKNWIPL

SEQ ID NO: 56 - protein, 307 aa, Chlamydia trachomatis
MRKIILCSPRGFCAGVIRAIQTVEVALEKWGRPIYVKHEIVHNRHVVDKLREKGAIFIEDLQEVPRNSRVIFSAHGVPPSVREEAEERGLIAIDATCGLVTKVHSAVKMYAKKGYHIILIGKRKHVEIIGIRGEAPDQITVVENIAEVEALPFSAQDPLFYVTQTTLSMDDAADIVAALKARYPRIFTLPSSSICYATQNRQGALRNILPQVDFVYVIGDTQSSNSNRLREVAERRGVTARLVNHPDEVTEEILQYSGNIGITAGASTPEDVVQACLMKLQELIPDLSIEMDLFVEEDTVFQLPKEL

SEQ ID NO: 57 - protein, 283 aa, Chlamydia trachomatis
MELNKTSESLFSAKIDHNHPRTEAHEPRDQREVRVFSLEGRSSTRQEKADRMPGRTSSRQESSKGSEEGAVHESTAGVSSKEEEESKGDGFFTGGNPTSGMALVETPMAVVSEAMVETSTMTVSQVDLQWVEQLVTSTVESLLVADIDGKQLVEIVLDNSNTVPAAFCGANLTLVQTGEEISVSFSNFVDQAQLTEATQLVQQNPKQLVSLVESLKARQLNLTELVVGNVAVSLPTIEKIETPLHMIAATIRHHDQEGDQEGEGRQDQHQGQHQEKKVEEAHI

SEQ ID NO: 58 - protein, 242 aa, Chlamydia trachomatis
MKVKINDQFICISPYISARWNQIAFIESCDGGTEGGITLKLHLIDGETVSIPNLGQAIVDEVFQEHLLYLESTAPQKNKEEEKISSLLGAVQQMAKGCEVQVFSQKGLVSMLLGGAGSINVLLQHSPEHKDHPDLPTDLLERIAQMMRSLSIGPTSILAKPEPHCNCLHCQIGRATVEEEDAGVSDEDLTFRSWDISQSGEKMYTVTDPLNPEEQFNVYLGTPIGCTCGQPYCEHVKAVLYT

SEQ ID NO: 59 - protein, 433 aa, Chlamydia trachomatis
MLIFALSFGADACLCAADLSKAKVEASVGDRAAFSPFTGEIKGNRVRLRLAPHTDSFIIKELSKGDCLAVLGESKDYYVVAAPEGVRGYVFRTFVLDNVIEGEKVNVRLEPSTSAPILARLSKGTVVKTLGAAQGKWIEIALPKQCVFYVAKNFVKNVGALDLYNQKEGQKKLALDLLSSAMDFADAELQKKIEDIDLDAIYKKMNLAQSEEFKDVPGLQSLVQKALERVQEAFLAKSLEKSSVKVPEIRHKVLEEIAVVSPAVEETPVVTKTEEQKVTTVPVPAPAVVTEPAQDLSSVKGSLLSHYIRKKGFVKASPVIEGRESFERSLFAVWLSLQPEEIRHQLTMESFYRDEQKKKRVLTGELEVYPHIVKNNPGDYLLKNGEDVVAFVYATSIDLSKWLGKSVVLECVSRPNNHFAFPAYIVLSVKEGA

SEQ ID NO: 60 - protein, 167 aa, Chlamydia trachomatis; identical to SEQ ID NO: 54

SEQ ID NO: 61 - protein, 194 aa, Chlamydia trachomatis; identical to SEQ ID NO: 53